(19) United States
(12) Patent Application Publication
(10) Pub. No.: US 2026/0274900 A1
(43) Pub. Date: Sep. 17, 2026

(54) SYSTEMS AND METHODS OF MAKING A POLYPEPTIDE COMPOSITION

(71) Applicant: Syngulon SRL, Charleroi (BE)

(72) Inventor: Philippe Gabant, Seraing (BE)

(21) Appl. No.: 19/408,191

(22) Filed: Dec. 3, 2025

Related U.S. Application Data
(63) Continuation of application No. PCT/US2024/032231, filed on Jun. 3, 2024.
(60) Provisional application No. 63/506,231, filed on Jun. 5, 2023.

(51) Int. Cl. C07K 14/195; C12N 15/10; G01N 33/487

(52) U.S. Cl. CPC C07K 14/195; C12N 15/1093; G01N 33/48721

(57) ABSTRACT

Provided herein is an in-vitro system for producing a specified mixture of polypeptides encoded by nucleic acids. Also provided is a method of selecting nucleic acids for producing a specified mixture of polypeptides encoded by the nucleic acids.

### REFERENCE TO RELATED APPLICATIONS

The present application is a continuation of International Patent Application No. PCT/US2024/032231, filed Jun. 3, 2024, which claims priority to U.S. Provisional Application No. 63/506,231, filed Jun. 5, 2023. The content of each of the aforementioned related application(s) is incorporated herein by reference in its entirety.

### REFERENCE TO SEQUENCE LISTING

The present application is being filed along with a Sequence Listing in electronic format. The Sequence Listing is provided as a file entitled SEQLIST_SYNSR_014C1.xml, created and last saved on Mar. 24, 2026, which is 865,215 bytes in size. The information in the electronic format of the Sequence Listing is incorporated herein by reference in its entirety.

### BACKGROUND

### Field

The present disclosure generally relates to in vitro polypeptide expression systems and methods.

A specified mixture of polypeptides, such as a mixture of antimicrobial peptides or bacteriocins, can be useful to provide a desired effect, such as bactericidal effects. Tuning the specified mixture of polypeptides to thereby tune the desired effect provided by the specified mixture may be useful in some cases.

### SUMMARY

Provided herein is an in-vitro system for producing a specified mixture of polypeptides encoded by nucleic acids, comprising: a membrane disposed between a first and second aqueous partitions, wherein the first aqueous partition comprises a plurality of nucleic acids encoding a plurality of different polypeptides, at least two of which correspond to polypeptide members of a specified mixture of two or more polypeptides, wherein the second aqueous partition comprises or is in fluid communication with a nucleic acid expression solution comprising at least a translation solution; an adaptive nucleic acid conduit comprising a nanopore disposed in the membrane such that the first and second aqueous partitions are in communication with each other via the nanopore, wherein the adaptive nucleic acid conduit is configured to selectively accept a nucleic acid molecule of the plurality of nucleic acids translocating through the nanopore into the second aqueous partition based on a detected sequence of the translocating nucleic acid molecule; and a selectivity controller configured to control the adaptive nucleic acid conduit's selectivity for accepting a nucleic acid molecule translocating through the nanopore from the first aqueous partition into the second aqueous partition, such that accepted nucleic acid molecules are allowed to be expressed in the nucleic acid expression solution to thereby generate of a mixture of two or more polypeptides encoded by the accepted nucleic acid molecules.

Also provided is a method of selecting nucleic acids for producing a specified mixture of polypeptides encoded by the nucleic acids, the method comprising: (a) providing in a first aqueous partition a plurality of nucleic acids encoding a plurality of different polypeptides, wherein the plurality of nucleic acids comprises nucleic acid molecules that encode at least two polypeptide members of a specified mixture of two or more polypeptides, wherein a membrane is disposed between the first aqueous partition and a second aqueous partition, wherein the first and second aqueous partitions are in communication with each other via a nanopore configured such that nucleic acid molecules of the plurality of nucleic acids can translocate from the first aqueous partition through the nanopore into the second aqueous partition; (b) sequencing a nucleic acid molecule of the plurality of nucleic acids translocating through the nanopore using nanopore-based sequencing to determine a nucleotide sequence of the translocating nucleic acid molecule; and (c) selectively accepting a nucleic acid molecule of the plurality of nucleic acids translocating through the nanopore into the second aqueous partition based on the determined nucleotide sequence, wherein the selectivity for accepting the translocating nucleic acid molecule into the second aqueous partition through the nanopore is controllable such that the accepted nucleic acid molecules, when expressed, produce a composition comprising the specified mixture of two or more polypeptides; and optionally (d) allowing the accepted nucleic acid molecule into the second aqueous partition to be expressed to generate a mixture of polypeptides encoded by the accepted nucleic acid molecules.

Embodiments of the present disclosure include the following numbered embodiments:
1 An in-vitro system for producing a specified mixture of polypeptides encoded by nucleic acids, comprising:
   a membrane disposed between a first and second aqueous partitions, wherein the first aqueous partition comprises a plurality of nucleic acids encoding a plurality of different polypeptides, at least two of which correspond to polypeptide members of a specified mixture of two or more polypeptides, wherein the second aqueous partition comprises or is in fluid communication with a nucleic acid expression solution comprising at least a translation solution;
   an adaptive nucleic acid conduit comprising a nanopore disposed in the membrane such that the first and second aqueous partitions are in communication with each other via the nanopore, wherein the adaptive nucleic acid conduit is configured to selectively accept a nucleic acid molecule of the plurality of nucleic acids translocating through the nanopore into the second aqueous partition based on a detected sequence of the translocating nucleic acid molecule; and
   a selectivity controller configured to control the adaptive nucleic acid conduit's selectivity for accepting a nucleic acid molecule translocating through the nanopore from the first aqueous partition into the second aqueous partition, such that accepted nucleic acid molecules are allowed to be expressed in the nucleic acid expression solution to thereby generate of a mixture of two or more polypeptides encoded by the accepted nucleic acid molecules.
2. The system of embodiment 1, wherein the selectivity controller allows the adaptive nucleic acid conduit to accept the translocating nucleic acid molecule into the second aqueous partition if at least the identity of the polypeptide encoded by the translocating nucleic acid molecule is a polypeptide member of the specified mixture, and does not allow the adaptive nucleic acid conduit to accept nucleic acid molecules into the second aqueous partition if the identity of the polypeptide encoded by the translocating nucleic acid molecule is not a polypeptide member of the specified mixture.
3. The system of embodiment 1 or 2, wherein the selectivity controller allows the adaptive nucleic acid conduit to accept the translocating nucleic acid molecule into the second aqueous partition if at least the identity of the polypeptide encoded by the translocating nucleic acid molecule corresponds to a polypeptide member that is deficient in a composition of polypeptides generated by expressing the accepted nucleic acid molecules compared to the specified mixture of two or more polypeptides.
4. The system of any one of the preceding embodiments, wherein the selectivity controller is configured to control the selectivity for accepting the nucleic acid molecule translocating through the nanopore into the second aqueous partition based on at least the specified mixture and an enumeration of the nucleic acid molecules encoding the polypeptide members of the specified mixture that have been accepted.
5. The system of any one of the preceding embodiments, wherein the selectivity controller is configured to control the selectivity for accepting the nucleic acid molecule translocation through the nanopore into the second aqueous partition such that the ratio of (i) a first nucleic acid encoding a first polypeptide corresponding to a first polypeptide member of the specified mixture and that is accepted into the second aqueous partition, and (ii) a second nucleic acid encoding a second polypeptide corresponding to a second member of the specified mixture and that is accepted into the second aqueous partition, is in proportion to the ratio of (iii) the molar amount of the first polypeptide member in the specified mixture, and (iv) the molar amount of the second polypeptide member of the specified mixture.
6. The system of any one of the preceding embodiments, wherein the selectivity controller is configured to set the rate at which nucleic acid molecules encoding polypeptide members of the specified mixture are accepted into the second aqueous partition.
7. The system of embodiment 6, wherein the rate at which nucleic acid molecules encoding polypeptide members of the specified mixture are accepted relative to each other is in proportion to the relative amount of the polypeptide members in the specified mixture.
8. The system of any one of the preceding embodiments, wherein the selectivity controller is configured to count the number of nucleic acid molecules encoding a polypeptide member of the specified mixture and that have been accepted into the second aqueous partition within a specified time period or a specified number of events.
9. The system of any one of the preceding embodiments, wherein the selectivity for accepting the nucleic acid molecule translocating through the nanopore into the second aqueous partition is altered based on the number of nucleic acid molecules that have been accepted into the second aqueous partition.
10. The system of any one of the preceding embodiments, wherein the selectivity for accepting the nucleic acid molecule is altered based on a ratio among nucleic acid molecules encoding two or more polypeptide members and that have been accepted into the second aqueous partition.
11. The system of any one of the preceding embodiments, wherein the system is configured to measure an amount and/or level of activity of the at least one polypeptide member in the mixture of polypeptides encoded by the accepted nucleic acid molecules.
12. The system of embodiment 11, wherein the selectivity for accepting the nucleic acid molecule is altered based on the measured amount and/or level of activity of the at least one polypeptide member in the mixture of polypeptides encoded by the accepted nucleic acid molecules.
13. The system of any one of the preceding embodiments, wherein the selectivity for accepting the nucleic acid molecule is altered based on a ratio of activity among, or a collective activity level of, two or more polypeptide members in the mixture of polypeptides encoded by the accepted nucleic acid molecules.
14. The system of any one of the preceding embodiments, wherein the adaptive nucleic acid conduit comprises a plurality of the nanopores, wherein the selectivity controller is configured to independently control the adaptive nucleic acid conduit's selectivity for accepting the nucleic acid molecule of the plurality of nucleic acids translocating through the two or more nanopores.
15. The system of any one of the preceding embodiments, wherein the plurality of nucleic acids comprises one or more nucleic acids that encode at least one polypeptide that does not correspond to a polypeptide member of the specified mixture.
16. The system of any one of the preceding embodiments, wherein the nucleic acid expression solution comprises a transcription solution.
17. The system of any one of the preceding embodiments, wherein the translocated nucleic acid molecules are transcribed in the nucleic acid expression solution.
18. The system of any one of the preceding embodiments, wherein the plurality of nucleic acids comprises RNA.
19. The system of any one of the preceding embodiments, wherein the plurality of nucleic acids comprises double-stranded DNA.
20. The system of any one of the preceding embodiments, wherein each of the plurality of nucleic acids comprises a barcode that identifies the polypeptide encoded by the nucleic acid molecule, wherein the adaptive nucleic acid conduit is configured to selectively accept the nucleic acid molecule into the second aqueous partition based on the detected sequence of the barcode.
21. The system of any one of the preceding embodiments, wherein the plurality of nucleic acids comprises a library of nucleic acids encoding different polypeptides of a functional class.
22. The system of any one of the preceding embodiments, wherein the plurality of different polypeptides comprises a plurality of different antimicrobial peptides.
23. The system of embodiment 22, wherein the antimicrobial peptides comprise bacteriocins.
24. The system of embodiment 22 or 23, wherein the specified mixture of two or more polypeptides targets an undesired microbial organism.
25. The system of any one of the preceding embodiments, wherein the second aqueous partition is in fluid communication with a tissue, a wound, a host microbiome, industrial culture, feedstock, fermenter, or a food, pharmaceutical, or cosmetic manufacturing environment.
26. The system of any one of the preceding embodiments, comprising a microfluidic device comprising the first and second aqueous partitions; the membrane disposed between the first and second aqueous partitions; and the nanopore disposed in the membrane.
27. The system of any one of the preceding embodiments, wherein the nanopore comprises MspA, alpha-hemolysin, anthrax toxin, leukocidins, OmpF, OmpG, OmpATb, NalP, and/or lysenin.
28. The system of any one of the preceding embodiments, wherein the adaptive nucleic acid conduit comprises a helicase or a polymerase associated with the nanopore.
29. The system of any one of the preceding embodiments, wherein the membrane comprises a lipid bilayer.
30. The system of any one of the preceding embodiments, wherein the first and second aqueous partitions are comprised in a chamber comprising the membrane disposed therein so as to separate the chamber into two portions, one of which comprises the first aqueous partition and the other comprises the second aqueous partition.
31. The system of any one of the preceding embodiments, wherein the specified mixture comprises a first polypeptide member and a second polypeptide member at a ratio of the first polypeptide member to the second polypeptide member of 1:2, 1:3, 1:4, 1:5, 1:6, 1:7, 1:8, 1:9, 1:10, 1:20, 1:30, 1:40, 1:50, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, 9:10, 19:20, 28:30, 29:30, 39:40, or 49:50.
32. The system of any one of the preceding embodiments, wherein the nucleic acid molecule can translocate from the first aqueous partition to the second aqueous partition only via the nanopore comprised in the adaptive nucleic acid conduit.
33. A method of selecting nucleic acids for producing a specified mixture of polypeptides encoded by the nucleic acids, the method comprising:
(a) providing in a first aqueous partition a plurality of nucleic acids encoding a plurality of different polypeptides, wherein the plurality of nucleic acids comprises nucleic acid molecules that encode at least two polypeptide members of a specified mixture of two or more polypeptides, wherein a membrane is disposed between the first aqueous partition and a second aqueous partition, wherein the first and second aqueous partitions are in communication with each other via a nanopore configured such that nucleic acid molecules of the plurality of nucleic acids can translocate from the first aqueous partition through the nanopore into the second aqueous partition;
(b) sequencing a nucleic acid molecule of the plurality of nucleic acids translocating through the nanopore using nanopore-based sequencing to determine a nucleotide sequence of the translocating nucleic acid molecule; and
(c) selectively accepting a nucleic acid molecule of the plurality of nucleic acids translocating through the nanopore into the second aqueous partition based on the determined nucleotide sequence, wherein the selectivity for accepting the translocating nucleic acid molecule into the second aqueous partition through the nanopore is controllable such that the accepted nucleic acid molecules, when expressed, produce a composition comprising the specified mixture of two or more polypeptides; and
optionally (d) allowing the accepted nucleic acid molecule into the second aqueous partition to be expressed to generate a mixture of polypeptides encoded by the accepted nucleic acid molecules.
34. The method of embodiment 33, wherein the second aqueous partition comprises or is in fluid communication with a nucleic acid expression solution comprising at least a translation solution, and wherein the method comprises allowing the accepted nucleic acid molecule in the second aqueous partition to contact the nucleic acid expression solution, whereby the accepted nucleic acid molecules in the nucleic acid expression solution are expressed to generate a mixture of polypeptides encoded by the accepted nucleic acid molecules and produce the specified mixture of two or more polypeptides.
35. The method of embodiment 33, comprising recombinantly expressing the accepted nucleic acid molecules to generate the mixture of polypeptides encoded by the accepted nucleic acid molecules.
36. The method of any one of embodiments 33-35, wherein selectively accepting the translocating nucleic acid molecule of the plurality of nucleic acids into the second aqueous partition comprises allowing the nucleic acid molecule to be accepted into the second aqueous partition if at least the identity of the polypeptide encoded by the translocating nucleic acid molecule corresponds to a polypeptide member of the specified mixture, while not allowing nucleic acid molecules to be accepted into the second aqueous partition if the identity of the polypeptide encoded by the translocating nucleic acid molecule is not a polypeptide member of the specified mixture.
37. The method of any one of embodiments 33-36, wherein selectively accepting the nucleic acid molecule of the plurality of nucleic acids comprises allowing the nucleic acid molecule to be accepted into the second aqueous partition if at least the identity of the polypeptide encoded by the translocating nucleic acid molecule corresponds to a polypeptide member that is deficient for the accepted nucleic acid molecules to generate the mixture of polypeptides when compared to the specified mixture of two or more polypeptides.
38. The method of any one of embodiments 33-37, comprising controlling the selectivity for accepting the nucleic acid molecule translocating through the nanopore into the second aqueous partition based on at least the specified mixture and an enumeration of the nucleic acid molecules encoding the polypeptide members of the specified mixture that have been accepted.
39. The method of any one of embodiments 33-38, comprising controlling the rate at which nucleic acid molecules encoding polypeptide members of the specified mixture are accepted into the second aqueous partition.
40. The method of embodiment 39, wherein the rate at which nucleic acid molecules encoding polypeptide members of the specified mixture are accepted relative to each other is in proportion to the relative amount of the polypeptide members in the specified mixture.
41. The method of any one of embodiments 33-40, comprising counting the number of nucleic acid molecules encoding a polypeptide member of the specified mixture and that have been accepted into the second aqueous partition within a specified time period or a specified number of events.
42. The method of any one of embodiments 33-41, comprising altering the selectivity for accepting the translocating nucleic acid molecule based on the number of nucleic acid molecules that have been accepted into the second aqueous partition.
43. The method of any one of embodiments 33-42, comprising altering the selectivity for accepting the translocating nucleic acid molecule based on a ratio among nucleic acid molecules encoding two or more polypeptide members and that have been accepted into the second aqueous partition.
44. The method of any one of embodiments 33-43, comprising measuring an amount and/or level of activity of the at least one polypeptide member in the generated mixture of polypeptides.
45. The method of embodiment 44, comprising altering the selectivity for accepting the translocating nucleic acid molecule based on the measured amount and/or level of activity of the at least one polypeptide member in the generated mixture of polypeptides.
46. The method of any one of embodiments 33-45, comprising altering the selectivity for accepting the translocating nucleic acid molecule based on a ratio among, or a collective activity level of, two or more polypeptide members in the generated mixture of polypeptides.
47. The method of any one of embodiments 33-46, comprising altering a composition of the specified mixture of polypeptides.
48. The method of embodiment 47, wherein altering the composition of the specified mixture of polypeptides comprises removing or replacing one or more polypeptides members of the specified mixture, altering an amount of one or more polypeptides members in the specified mixture, and/or altering a proportion of one or more polypeptides members in the specified mixture.
49. The method of any one of embodiments 33-48, wherein the first and second aqueous partitions are in communication with each other via a plurality of the nanopores, wherein the method comprises:
   determining a nucleotide sequence of the nucleic acid molecule translocating through each of two or more nanopores of the plurality of the nanopores; and
   selectively accepting the nucleic acid molecule translocating through each of the two or more nanopores based on the determined nucleotide sequence of the corresponding translocating nucleic acid molecule.
50. The method of any one of embodiments 33-49, wherein the plurality of nucleic acids comprises one or more nucleic acids that encode at least one polypeptide that does not correspond to a polypeptide member of the specified mixture.
51. The method of any one of embodiments 33-50, wherein the nucleic acid expression solution further comprises a transcription solution.
52. The method of any one of embodiments 33-51, comprising allowing the accepted nucleic acid molecule in the second aqueous partition to be transcribed in the nucleic acid expression solution.
53. The method of any one of embodiments 33-52, wherein the plurality of nucleic acids comprises RNA.
54. The method of any one of embodiments 33-53, wherein the plurality of nucleic acids comprises double-stranded DNA.
55. The method of any one of embodiments 33-54, wherein each of the plurality of nucleic acids comprises a barcode that identifies the polypeptide encoded by the nucleic acid molecule,
wherein the determined nucleotide sequence comprises the barcode.
56. The method of any one of embodiments 33-55, wherein the plurality of nucleic acids comprises a library of nucleic acids encoding different polypeptides of a functional class.
57. The method of any one of embodiments 33-56, wherein the plurality of different polypeptides comprises a plurality of different antimicrobial peptides.
58. The method of embodiment 57, wherein the antimicrobial peptides comprise bacteriocins.
59. The method of embodiment 56 or 57, wherein the specified mixture of two or more polypeptides targets an undesired microbial organism.
60. The method of any one of embodiments 33-59, wherein the second aqueous partition is in fluid communication with a tissue, a wound, a host microbiome, industrial culture, feedstock, fermenter, or a food, pharmaceutical, or cosmetic manufacturing environment.
61. The method of any one of embodiments 33-60, wherein the first and second aqueous partitions, the membrane disposed between the first and second aqueous partitions, and the nanopore disposed in the membrane are comprised in a microfluidic device.
62. The method of any one of embodiments 33-61, the nanopore comprises MspA, alpha-hemolysin, anthrax toxin, leukocidins, OmpF, OmpG, OmpATb, NalP, and/or lysenin.
63. The method of any one of embodiments 33-62, wherein the nanopore is associated with a helicase or a polymerase.
64. The method of any one of embodiments 33-63, wherein the membrane comprises a lipid bilayer.
65. The method of any one of embodiments 33-64, wherein the first and second aqueous partitions are comprised in a chamber comprising the membrane disposed therein so as to separate the chamber into two portions, one of which comprises the first aqueous partition and the other comprises the second aqueous partition.
66. The method of any one of embodiments 33-65, wherein the specified mixture comprises a first polypeptide member and a second polypeptide member at a ratio of the first polypeptide member to the second polypeptide member of 1:2, 1:3, 1:4, 1:5, 1:6, 1:7, 1:8, 1:9, 1:10, 1:20, 1:30, 1:40, 1:50, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, 9:10, 19:20, 28:30, 29:30, 39:40, or 49:50.
67. The method of any one of embodiments 33-66, wherein the nucleic acid molecule can translocate from the first aqueous partition to the second aqueous partition only via the nanopore comprised in the adaptive nucleic acid conduit.
68. The method of any one of embodiments 33-67, comprising using the system of any one of embodiments 1-32.

### BRIEF DESCRIPTION OF THE DRAWINGS

FIG. 1 is a schematic diagram showing a non-limiting embodiment of a system of the present disclosure.

FIG. 2 is a block diagram showing a non-limiting embodiment of a method of the present disclosure.

### DETAILED DESCRIPTION

Provided herein are systems and methods for making a polypeptide composition having a specified mixture of polypeptides. Some embodiments include methods and systems for generating compositions comprising polypeptides in precise ratios or stoichiometries, which can be useful for tuning the effect of the composition for specific goals. In some embodiments, compositions comprising antimicrobial peptides and bacteriocins in precise ratios or stoichiometries can be useful for tuning a population of microbial organisms in a number of applications, for example in industrial biotechnology manufacturing processes, pharmaceutical, biologic, and cosmetic manufacturing, and medical applications. Some embodiments include methods and systems that rely on nanopore-based sequencing to identify nucleic acid molecules (e.g., DNA, RNA) that encode polypeptide members of the specified mixture of polypeptides among a plurality of nucleic acids, and selectively translocate nucleic acid molecules of interest into a partition such that the translocated nucleic acids, when expressed, can generate a composition having the specified mixture of polypeptides. In some embodiments, the systems and methods herein allow for tuning of the selectivity in real-time, to achieve translocation of nucleic acids encoding the relevant polypeptides in the desired amounts and stoichiometry, and to adjust the specified mixture, for example, if a different specified mixture is desired due to change in conditions.

Unless stated otherwise, terms used herein have their customary and ordinary meaning as understood by one of skill in the art in view of this disclosure.

### Systems

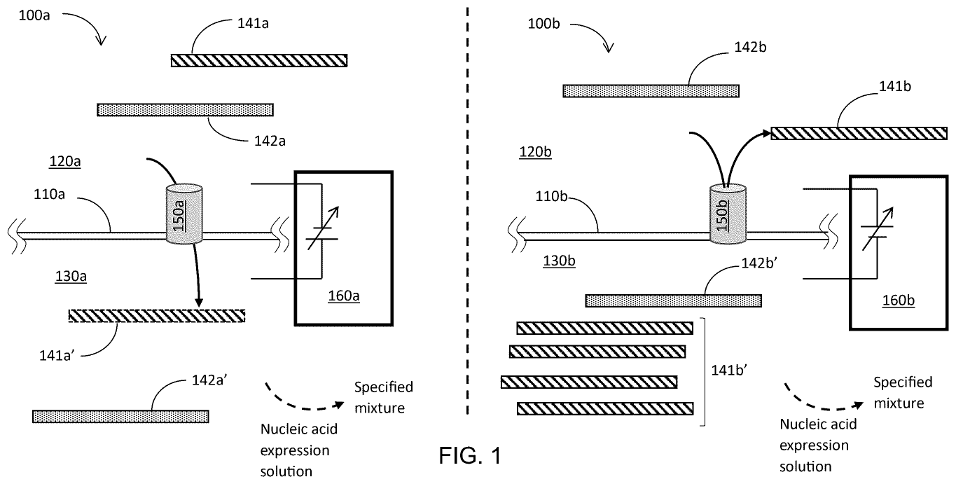

With reference to FIG. 1 (left panel), a schematic diagram of an in-vitro system for producing a specified mixture of polypeptides encoded by nucleic acids is provided. The system 100a can include a membrane 110a disposed between a first 120a and second 130a aqueous partitions, wherein the first aqueous partition comprises a plurality of nucleic acids (e.g., 141a, 142a) encoding a plurality of different polypeptides, at least two of which correspond to polypeptide members of a specified mixture of two or more polypeptides, wherein the second aqueous partition comprises or is in fluid communication with a nucleic acid expression solution comprising at least a translation solution. The system 100a can also include an adaptive nucleic acid conduit comprising a nanopore 150a disposed in the membrane such that the first and second aqueous partitions are in communication (e.g., fluid communication) with each other via the nanopore, wherein the adaptive nucleic acid conduit is configured to selectively accept a nucleic acid molecule of the plurality of nucleic acids translocating through the nanopore into the second aqueous partition based on a detected sequence of the translocating nucleic acid molecule. The system 110a can further include a selectivity controller 160a configured to control the adaptive nucleic acid conduit's selectivity for accepting a nucleic acid molecule translocating through the nanopore from the first aqueous partition into the second aqueous partition, such that accepted nucleic acid molecules (e.g., 141a′) are allowed to be expressed in the nucleic acid expression solution, to thereby generate of a mixture of two or more polypeptides encoded by the accepted nucleic acid molecules. In some embodiments, the adaptive nucleic acid conduit comprises the selectivity controller. In some embodiments, the membrane 110a disposed between the first 120a and second 130a aqueous partitions prevents translocation of any of the plurality of nucleic acids between the first and second aqueous partitions, such that nucleic acids can only translocate from the first to the second aqueous partition, and/or from the second to the first aqueous partition, via one or more adaptive nucleic acid conduit(s) comprising a nanopore(s) disposed in the membrane. In some embodiments, nucleic acids can translocate from the first to the second aqueous partition only via one or more adaptive nucleic acid conduit(s) comprising a nanopore(s) disposed in the membrane; the adaptive nucleic acid conduit(s) comprising a nanopore(s) do not permit translocation of nucleic acids from the second to the first aqueous partition.

As used herein “selectively accept a nucleic acid molecule of the plurality of nucleic acids translocating through the nanopore into the second aqueous partition” denotes that upon determining that a nucleic acid molecule translocating through the nanopore encodes a polypeptide member of the specified mixture of two or more polypeptides that is desired to be expressed, the adaptive nucleic acid conduit allows the translocating nucleic acid molecule to complete translocation into the second aqueous partition, and upon determining that a nucleic acid molecule translocating through the nanopore encodes a polypeptide that is not desired to be expressed, the adaptive nucleic acid conduit prevents the translocating nucleic acid molecule from completing translocation into the second aqueous partition (or “bounces out” the translocating nucleic acid molecule back to the first aqueous partition).

In some embodiments, the adaptive nucleic acid conduit comprises a plurality of the nanopores, wherein the selectivity controller is configured to independently control the adaptive nucleic acid conduit's selectivity for translocation of the nucleic acid molecule of the plurality of nucleic acids through the two or more nanopores. Any suitable number of nanopores may be disposed on the membrane. In some embodiments, the adaptive nucleic acid conduit comprises at least, or at least about 20, 50, 100, 200, 500, 750, 1,000, 1,250, 1,500, 1,750, 2,000, 2,500, 3,000, 4,000, 5,000, or more, or a number in a range defined any two of the preceding values (e.g., 20-5,000, 100-4,000, 1,500-2,500, 1,750-3,000, etc.) nanopores.

In some embodiments, the system includes structural elements to measure and/or apply an electrical potential across the nanopore-bearing membrane (e.g., to implement the adaptive nucleic acid conduit). For example, the system can include a pair of drive electrodes that drive current through the nanopores. The system can be configured so that the negative pole and positive pole can be adjusted. In some embodiments, the system is configured to adjust the magnitude and/or polarity of the electrical potential across the membrane. In some embodiments, the nucleic acid input region (e.g., the first aqueous partition 120a) is the negative pole and the output region (e.g., the second aqueous partition 130a) is the positive pole. In this arrangement, the nucleic acid can translocate from the first aqueous partition into the second aqueous partition through the nanopore (e.g., the translocating nucleic acid molecule is accepted into the second aqueous partition). In some embodiments, the polarity is switched such that the nucleic acid input region (e.g., the first aqueous partition 120a) is the positive pole and the output region (e.g., the second aqueous partition 130a) is the negative pole. In this arrangement, the nucleic acid is prevented from translocating from the first aqueous partition into the second aqueous partition through the nanopore (e.g., a translocating nucleic acid molecule is bounced out). In some embodiments, the selectivity controller sets the selectivity rule under which the adaptive nucleic acid conduit determines whether a nucleic acid molecule translocating through the nanopore is accepted or bounced out based on the detected sequence.

In some embodiments, the system includes one or more measurement electrodes that measure the current through the nanopore (e.g., to implement the adaptive nucleic acid conduit). These can include, for example, a patch-clamp amplifier or a data acquisition device. For example, nanopore systems can include an Axopatch-IB patch-clamp amplifier (Axon Instruments, Union City, CA) to apply voltage across the bilayer and measure the ionic current flowing through the nanopore. For example, in some embodiments, the applied electrical field includes a direct or constant current that is between about 10 mV and about 1 V. In some embodiments that include protein-based nanopores embedded in lipid membranes, the applied current includes a direct or constant current that is between about 10 mV and 300 mV, such as about 10 mV, 20 mV, 30 mV, 40 mV, 50 mV, 60 mV, 70 mV, 80 mV, 90 mV, 100 mV, 110 mV, 120 mV, 130 mV, 140 mV, 150 mV, 160 mV, 170 mV, 180 mV, 190 mV, 200 mV, 210 mV, 220 mV, 230 mV, 240 mV, 250 mV, 260 mV, 270 mV, 280 mV, 290 mV, 300 mV, or any voltage therein. In some embodiments, the applied electrical field is between about 40 mV and about 200 mV. In some embodiments, the applied electrical field includes a direct or constant current that is between about 100 mV and about 200 mV. In some embodiments, the applied electrical direct or constant current field is about 180 mV. In other embodiments where solid state nanopores are used, the applied direct or constant current electrical field can be in a similar range as described, up to as high as 1 V. In some instances, the electrical potential applied can be sufficient to translocate a nucleic acid through the nanopore. In some embodiments, the sequence of nucleotides passing through the nanopore is detected by detecting the change in the current through the nanopore.

In some embodiments, the system is configured to control the magnitude and/or polarity of the electrical potential across the membrane, based on the detected sequence (e.g., partial sequence) of the translocating nucleic acid (e.g., to implement the adaptive nucleic acid conduit). In some embodiments, the system is configured to enrich for and/or increase representation of nucleic acids encoding polypeptide members of the specified mixture of two or more polypeptides using adaptive sampling of the input nucleic acids (e.g., in the first aqueous partition) via the adaptive nucleic acid conduit. In some embodiments, the selectivity controller allows the adaptive nucleic acid conduit to accept the translocating nucleic acid molecule into the second aqueous partition if at least the identity of the polypeptide encoded by the translocating nucleic acid molecule is a polypeptide member of the specified mixture, and prevents translocation of nucleic acid molecules from the first aqueous partition into the second aqueous partition if the identity of the polypeptide encoded by the translocating nucleic acid molecule is not a polypeptide member of the specified mixture. In some embodiments, the specified mixture of two or more polypeptides is defined by the identities of the polypeptide members in the specified mixture (e.g., as provided by a list or table that includes the polypeptide members in the specified mixture). In some embodiments, the specified mixture of two or more polypeptides is defined by the identities of the polypeptide members in the specified mixture, without specifying the amount or relative amount of each polypeptide member in the specified mixture.

For example, in FIG. 1, left panel, a nucleic acid 141a that encodes a polypeptide member of the specified mixture of two or more polypeptides can enter the nanopore 150a and start translocating, as the polarity of the electrical potential is set to be positive on the second aqueous partition 130a side and negative on the first aqueous partition 120a side. The system can be configured to detect the nucleotide sequence of the translocating nucleic acid molecule as it is translocating through the nanopore. Any suitable nanopore-based sequencing option can be used. When the translocating nucleic acid molecule is identified as a polypeptide member of the specified mixture, the selectivity controller 160a can allow the adaptive nucleic acid conduit to accept the translocating nucleic acid molecule into the second aqueous partition 130a (e.g., the adaptive nucleic acid conduit does not change the polarity of the electrical potential applied across the membrane).

The first aqueous partition 120a, 120b can include any suitable collection of nucleic acids, as described herein. In some embodiments, the nucleic acids encoding polypeptide members of the specified mixture in the first aqueous partition are not in the proportion according to the specified mixture.

The translocating nucleic acid molecule can be identified to encode a polypeptide that corresponds to a polypeptide member of the specified mixture using any suitable option. In some embodiments, the amino acid sequence of the polypeptide encoded by the sequenced portion of the translocating nucleic acid molecule is aligned with the amino acid sequences of the polypeptide members of the specified mixture.

In some embodiments, the system is configured to sequence (e.g., via nanopore sequencing) any suitable length of the translocating nucleic acid molecule to determine whether the polypeptide encoded by the translocating nucleic acid molecule corresponds to a polypeptide member of the specified mixture. In some embodiments, the minimum length of the translocating nucleic acid molecule that is sequenced depends on the level of sequence diversity among the plurality of nucleic acids in the first aqueous partition. In some embodiments, at least, or at least about 18, 21, 24, 27, 30, 40, 50, 60, 70, 80, 90, 100, 120, 140, 160, 180, or at least 200 nucleotides, or at most, or at most about 300, 250, 200, 150, 100, 80, or at most 60 nucleotides, or a length in a range defined by any two of the preceding values (e.g., about 18-300, 30-250, 60-200, 18-100, etc.) of the translocating nucleic acid molecule is sequenced to determine whether the polypeptide encoded by the translocating nucleic acid molecule corresponds to a polypeptide member of the specified mixture.

In some embodiments, the specified mixture of two or more polypeptides is defined by the identities of the polypeptide members in the specified mixture (e.g., as provided by a list or table that includes the polypeptide members in the specified mixture), and an amount of one or more of the polypeptide members, and/or a ratio (or stoichiometry) of the polypeptide members relative to each other. As used herein, “ratio” or “stoichiometry” denote the relative amount of two or more molecules expressed in the number of molecules or in molar quantities.

In some embodiments, the selectivity controller is configured to control the selectivity for translocation of the nucleic acid molecule into the second aqueous partition through the nanopore such that the ratio of (i) a first nucleic acid molecule encoding a first polypeptide corresponding to a first polypeptide member of the specified mixture of two or more polypeptides and that is accepted into the second aqueous partition, and (ii) a second nucleic acid molecule encoding a second polypeptide corresponding to a second member of the specified mixture of two or more polypeptides and that is accepted into the second aqueous partition is in proportion to the ratio of (iii) the molar amount of the first polypeptide member in the specified mixture of two or more polypeptides, and (iv) the molar amount of the second member of the specified mixture of two or more polypeptides.

In some embodiments, the system is configured to control the rates at which nucleic acid molecules encoding each of the polypeptide members of the specified mixture are accepted into the second aqueous partition. In some embodiments, the selectivity controller is configured to set the rate at which nucleic acid molecules encoding polypeptide members of the specified mixture are accepted into the second aqueous partition. In some embodiments, the rate at which nucleic acid molecules encoding polypeptide members of the specified mixture are accepted into the second aqueous partition is measured over a suitable time interval. In some embodiments, the rate is measured over about 0.1-0.5 seconds, about 0.5-1 seconds, about 1-5 seconds, or about 5-10 seconds, or longer. In some embodiments, the selectivity controller is configured to count the number of nucleic acid molecules encoding a polypeptide member of the specified mixture and that have been accepted into the second aqueous partition within a specified time period or a specified number of events (e.g., the number of one or more other nucleic acids encoding a polypeptide member of the specified mixture that is accepted). In some embodiments, the number of nucleic acid molecules that have been accepted into the second aqueous partition is counted at specified time intervals (e.g., every 0.1-0.5 second, every 0.5-1 seconds, every 1-5 seconds, every 5-10 seconds, or longer). In some embodiments, the number of nucleic acid molecules that have been accepted into the second aqueous partition is counted at an interval defined by a specified number of events. For example, the number of nucleic acid molecules encoding polypeptide A of the specified mixture that have been accepted is counted for every 10 nucleic acid molecules encoding polypeptide B of the specified mixture that have been accepted.

In some embodiments, the rate at which nucleic acid molecules encoding polypeptide members of the specified mixture are accepted relative to each other is in proportion to the relative amount of the polypeptide members in the specified mixture. For example, where the specified mixture includes polypeptide A and polypeptide B at a relative concentration of 1:3, the selectivity controller controls the selectivity of the adaptive nucleic acid conduit such that the nucleic acid molecules encoding polypeptide B is accepted into the second aqueous partition at a rate three times faster than and the rate at which nucleic acid molecules encoding polypeptide A are accepted. In some embodiments, the rate at which nucleic acid molecules encoding polypeptide members of the specified mixture are accepted into the second aqueous partition is measured based on the number of times a nucleic acid molecule encoding a polypeptide member of the specified mixture is accepted into the second aqueous partition relative to the number of times a nucleic acid molecule encoding another polypeptide member of the specified mixture is accepted into the second aqueous partition. For example, where the specified mixture includes polypeptide A and polypeptide B at a relative concentration of 1:3, the selectivity controller controls the selectivity of the adaptive nucleic acid conduit such that for every three nucleic acid molecules encoding polypeptide B that is accepted into the second aqueous partition, one nucleic acid molecule encoding polypeptide A is accepted.

As a further example and not to be limiting, the first aqueous partition may include nucleic acid molecules encoding polypeptides in a specified mixture that includes A and B, which may be a ratio of 1:2 of A to B. The first aqueous partition may also include other nucleic acid molecules encoding polypeptides that are not in the specified mixture (e.g., C and D). The nucleic acid molecules may be in equal proportion (or in an unspecified proportion) in the first aqueous partition. In the beginning, the selectivity of the adaptive nucleic acid conduit may allow nucleic acid molecules encoding A or B to be accepted into the second aqueous partition, while those encoding C or D are not accepted. In one non-limiting option, nucleic acids encoding A or B are accepted at equal rate (e.g., with no selectivity between the two), once the desired minimum amount of nucleic acid molecules encoding A is accepted into the second aqueous partition, the selectivity controller can adjust the selectivity of the adaptive nucleic acid conduit to no longer accept nucleic acid molecules encoding A and continue to accept nucleic acid molecules encoding B, until the desired ratio of nucleic acid molecules are accepted.

In some embodiments, the system is configured to dynamically control selectivity of the adaptive nucleic acid conduit for accepting a nucleic acid molecule translocating through the nanopore from the first aqueous partition into the second aqueous partition. In some embodiments, the system is configured to dynamically control selectivity of the adaptive nucleic acid conduit for accepting a nucleic acid molecule translocating through the nanopore from the first aqueous partition into the second aqueous partition across time, events, and/or across space (e.g., where a plurality of nanopores used). In some embodiments, dynamically controlling the selectivity of the adaptive nucleic acid conduit includes continuously determining whether the selectivity of the adaptive nucleic acid conduit needs to be altered. In some embodiments, dynamically controlling the selectivity of the adaptive nucleic acid conduit includes determining at a defined time interval (e.g., over about 0.1-0.5 seconds, about 0.5-1 seconds, about 1-5 seconds, or about 5-10 seconds, or longer) whether to alter the selectivity of the adaptive nucleic acid conduit. In some embodiments, dynamically controlling the selectivity of the adaptive nucleic acid conduit includes determining after a defined number of events (e.g., after about 1-5, about 5-10, about 10-50, about 50-100, about 100-500, about 500-1000, or more events) whether to alter the selectivity of the adaptive nucleic acid conduit.

In some embodiments, dynamically controlling the selectivity of the adaptive nucleic acid conduit includes changing the selectivity while the system continues to translocate nucleic acids (e.g., to achieve the specified mixture). In some embodiments, the selectivity for translocation of the nucleic acid molecule is altered based on the number of nucleic acid molecules that have translocated into the second aqueous partition. In some embodiments, dynamically controlling the selectivity of the adaptive nucleic acid conduit includes changing the selectivity based on a difference in the desired amount or ratio of nucleic acid molecules encoding the polypeptide members of the specified mixture and the amount or ratio of accepted nucleic acid molecules encoding the polypeptide members of the specified mixture. In some embodiments, the system (e.g., the selectivity controller) is configured to enumerate the nucleic acid molecules that have been accepted. In some embodiments, the system (e.g., the selectivity controller) is configured to enumerate the identity and/or the amount of the nucleic acid molecules that have been accepted. In some embodiments, the selectivity controller is configured to control the selectivity for accepting the nucleic acid molecule translocating through the nanopore into the second aqueous partition based on at least the specified mixture (e.g., the composition of polypeptides in the specified mixture) and an enumeration of the nucleic acid molecules encoding the polypeptide members of the specified mixture that have been accepted. As used herein “enumerate” denotes at least counting the number of events or items (e.g., the number of individual nucleic acid molecules that have been accepted, the number of times acceptance of an individual nucleic acid molecule has occurred been accepted, etc.). In some embodiments, enumerating includes generating a list or a record of the number of events or items counted. In some embodiments, enumerating is done at a specified time interval or at an interval defined by a specified number of events. In some embodiments, the selectivity for translocation of the nucleic acid molecule is altered based on a ratio among nucleic acid molecules encoding two or more polypeptide members and that have translocated into the second aqueous partition.

In some embodiments, the selectivity controller allows the adaptive nucleic acid conduit to accept the translocating nucleic acid molecule into the second aqueous partition if at least the identity of the polypeptide encoded by the translocating nucleic acid molecule corresponds to a polypeptide member that is deficient in a composition of polypeptides generated by expressing the accepted nucleic acid molecules compared to the specified mixture of two or more polypeptides. In some embodiments, a composition of polypeptides generated by expressing the accepted nucleic acid molecules is expected to be deficient by lacking one or more polypeptides members that are in the specified mixture. In some embodiments, a composition of polypeptides generated by expressing the accepted nucleic acid molecules is expected to be deficient by having an insufficient amount of the one or more polypeptides members that are in the specified mixture. In some embodiments, a composition of polypeptides generated by expressing the accepted nucleic acid molecules is expected to be deficient by having an insufficient amount of the one or more polypeptides members that are in the specified mixture relative to one or more other polypeptides members that are in the specified mixture. In some embodiments, a composition of polypeptides generated by expressing the accepted nucleic acid molecules is expected to be deficient by having an excess of the one or more polypeptides members that are in the specified mixture. In some embodiments, a composition of polypeptides generated by expressing the accepted nucleic acid molecules is expected to be deficient by having an excess of the one or more polypeptides members that are in the specified mixture relative to one or more other polypeptides members that are in the specified mixture. In some embodiments, a composition of polypeptides generated by expressing the accepted nucleic acid molecules is expected to be deficient where the composition of the specified mixture has been altered or updated (e.g., by removing or replacing one or more polypeptides members of the specified mixture, altering an amount of one or more polypeptides members in the specified mixture, and/or altering a proportion of one or more polypeptides members in the specified mixture). In some embodiments, dynamically controlling the selectivity of the adaptive nucleic acid conduit includes changing the selectivity rule (e.g., provided by the selectivity controller) under which the adaptive nucleic acid conduit determines whether a nucleic acid molecule translocating through the nanopore is accepted or bounced out based on the detected sequence, from a first selectivity rule to a second selectivity rule, upon altering or updating the composition of the specified mixture.

For example, in FIG. 1, left panel, the specified mixture may include a first polypeptide member encoded by a first nucleic acid molecule 141a, and a second polypeptide member encoded by a second nucleic acid molecule 142a. A second nucleic acid encoding the second polypeptide member has been accepted 142a′ into the second aqueous partition 130a. When a first nucleic acid molecule 141a is translocating through the nanopore 150a, the translocating nucleic acid molecule is determined to encode the first polypeptide member based on the detected sequence of the translocating nucleic acid molecule. A composition of polypeptides generated by expressing the accepted nucleic acids (before the currently translocating nucleic acid molecule 141a is completely in the second aqueous partition) is expected to be deficient in the first polypeptide member, and the selectivity controller allows the translocating nucleic acid molecule to be accepted into the second aqueous partition 130a (by allowing the polarity of the voltage applied across the membrane to be maintained).

By way of another example, in FIG. 1, right panel, the specified mixture may include a first polypeptide member encoded by a first nucleic acid molecule 141b, and a second polypeptide member encoded by a second nucleic acid molecule 142b, and may further include the first and second polypeptide members at a ratio of 2:1 of the first and second polypeptide members, respectively. First nucleic acids 141b′ encoding the first polypeptide member and second nucleic acids 142b′ encoding the second polypeptide member have been accepted into the second aqueous partition 130b at a ratio of 4:1 of the first and second nucleic acids, respectively. When a first nucleic acid molecule 141b is translocating through the nanopore 150b, the translocating nucleic acid molecule is determined to encode the first polypeptide member based on the detected sequence of the translocating nucleic acid molecule. A composition of polypeptides generated by expressing the accepted nucleic acids (before the currently translocating nucleic acid molecule 141b is completely in the second aqueous partition) is expected to have an excess of the first polypeptide member, and the selectivity controller does not allow the translocating nucleic acid molecule to be accepted into the second aqueous partition 130b (e.g., by causing the polarity of the voltage applied across the membrane to be reversed).

In some embodiments, the system is configured to dynamically control the rates at which nucleic acid molecules encoding each of the polypeptide members of the specified mixture are accepted into the second aqueous partition. For example and not to be limiting, Table 0.1 shows dynamic control of selectivity. The system input includes nucleic acids x, y and z, each encoding the respective polypeptides. The specified mixture may include polypeptides encoded by nucleic acids x and y, in amounts corresponding to the amount of each generated by expressing 200 AU (arbitrary units) of polypeptide x, 400 AU of polypeptide y. In run 1, no nucleic acid has been accepted, and the selectivity of the adaptive nucleic acid conduit is set to accept nucleic acids at a ratio of 1:2:0 of x:y:z. In run 2, the nucleic acids accepted are x=100, and y=100 (and no z). The selectivity of the adaptive nucleic acid conduit is set to accept nucleic acids at a ratio of 1:3:0 of x:y:z. In run 3, the nucleic acids accepted are x=200, and y=200 (and no z). As no more nucleic acid x is required, the selectivity of the adaptive nucleic acid conduit is set to accept nucleic acids at a ratio of 0:1:0 of x:y:z.

TABLE 0.1

| | Target amount of nucleic | | | |
|---|---|---|---|---|
| | acids to generate specified | | | Selectivity (ratio of rates |
| | mixture (amount in | | | of accepting x, y, z, |
| Input | arbitrary units) | Run | Number Accepted | respectively) |
| x, y, z | x = 200, y = 400, z = 0 | 1 | x = 0, y = 0, z = 0 | 1:2:0 |
| | | 2 | x = 100, y = 100, z = 0 | 1:3:0 |
| | | 3 | x = 200, y = 200, z = 0 | 0:1:0 |

In some embodiments, controlling the selectivity of the adaptive nucleic acid conduit includes changing the selectivity based on a update of the current state of the system. In some embodiments, controlling the selectivity of the adaptive nucleic acid conduit includes changing the selectivity based on one or more feedbacks to the system.

In some embodiments, the specified mixture of the two or more polypeptides exhibits a desired activity (e.g., antimicrobial activity, bactericidal activity, enzymatic activity, inhibitory activity, toxicity, etc.). In some embodiments, the selectivity of the adaptive nucleic acid conduit is altered based on a measured activity level of one or more polypeptides in the specific mixture that is generated by expressing the accepted nucleic acids. In some embodiments, the system is configured to measure an amount and/or level of activity of the at least one polypeptide member in the mixture of polypeptides encoded by the accepted nucleic acid molecules. Any suitable activity of the at least one polypeptide member in the mixture of polypeptides encoded by the accepted nucleic acid molecules can be measured. In some embodiments, the activity is antimicrobial activity, bactericidal activity, enzymatic activity, inhibitory activity, toxicity, signaling, etc.). In some embodiments, the activity is an antimicrobial activity or bactericidal activity. In some embodiments, the activity is signaling.

In some embodiments, the selectivity controller is configured to alter the selectivity for accepting the nucleic acid molecule based on the measured amount and/or level of activity of the at least one polypeptide member in the mixture of polypeptides encoded by the accepted nucleic acid molecules. In some embodiments, the selectivity controller is configured to alter the selectivity of the adaptive nucleic acid conduit to increase the rate or amount of accepting a nucleic acid where the measured activity level of a polypeptide member in the mixture of polypeptides encoded by the nucleic acid molecules is lower than a desired level. In some embodiments, the selectivity controller is configured to alter the selectivity of the adaptive nucleic acid conduit to reduce the rate or amount of accepting (or prevent accepting) a nucleic acid where the measured activity level of a polypeptide member in the mixture of polypeptides encoded by the nucleic acid molecules is higher than a desired level. In some embodiments, the selectivity for the nucleic acid molecule is altered based on a ratio of activity among, or a collective activity level of, two or more polypeptide members in the mixture of polypeptides encoded by the translocated nucleic acid molecules.

In some embodiments, the system includes a processor and non-transient memory comprising instructions, which when executed, causes the processor to control one or more other components of the system, as provided herein. In some embodiments, the instructions, when executed, causes the processor to sequence a translocating nucleic acid, control polarity of the voltage applied across the membrane, and/or control the adaptive nucleic acid conduit's selectivity for accepting the adaptive nucleic acid conduit's selectivity for accepting a nucleic acid molecule translocating through the nanopore from the first aqueous partition into the second aqueous partition. In some embodiments, the instructions, when executed, causes the processor to perform the method of some embodiments described herein.

In systems, methods, and kits of some embodiments herein, the second aqueous partition is in fluid communication with a tissue, a wound, a host microbiome, industrial culture, feedstock, fermenter, or a food, pharmaceutical, or cosmetic manufacturing environment. In some embodiments, the specified mixture can flow to a tissue, wound, microbiome of a subject, and/or vessel. In some embodiments, a microfluidic device comprising the first and second aqueous partitions; the membrane disposed between the first and second aqueous partitions; and the nanopore disposed in the membrane, can be used. In some embodiments, the microfluidic device includes one or more valves to control fluid flow between different compartments.

In accordance with the methods, systems and kits of some embodiments described herein, the first aqueous partition and the second aqueous partition each has a volume of no more than 1, 5, 10, 20, 50, 100, 250 or 500 microliters, including ranges between any two of the listed values, for example, 1-5 microliters, 1-10 microliters, 1-20 microliters, 1-50 microliters, 1-100 microliters, 1-500 microliters, 5-10 microliters, 5-20 microliters, 5-50 microliters, 5-100 microliters, 5-500 microliters, 10-20 microliters, 10-50 microliters, 10-100 microliters, 10-500 microliters, 50-100 microliters, or 50-500 microliters. In some embodiments, the aqueous partitions are housed within a chamber that comprise, consist essentially of, or consist of a material or product selected from the group consisting of a well, microwell, nanowell, membrane, matrix, plastic, metal, glass, polymer, polysaccharide, and paramagnetic compound, or a combination of two or more of these. In some embodiments, the first and second aqueous partitions are comprised in a chamber (or well, etc.) comprising the membrane disposed therein so as to separate the chamber (or well, etc.) into at least two portions, one of which comprises the first aqueous partition and the other comprises the second aqueous partition.

### Membranes, Nanopores, and Adaptive Nucleic Acid Conduit

In systems, methods, and kits of some embodiments, the membrane can be any suitable membrane. In some embodiments, the membrane is a film. In some embodiments, the membrane is substantially flat. In some embodiments, the membrane is flexible. In some embodiments, the membrane is a lipid bilayer. Suitable lipid bilayers include, but are not limited to, a planar lipid bilayer, a supported bilayer or a liposome. In some embodiments, the membrane lipid bilayer is a planar lipid bilayer. In some embodiments, the membrane includes a phospholipid bilayer. In some embodiments, the membrane comprises a block copolymer.

Systems, methods, and kits of some embodiments comprise an adaptive nucleic acid conduit configured to selectively translocate nucleic acids through the nanopore using any suitable option. A “nanopore” as used herein denotes a pore typically having a size of the order of nanometers that allows the passage of a nucleic acid molecule (e.g., DNA, RNA), therethrough. In some embodiments, a nanopore has an opening with a diameter at its most narrow point of about 0.3 nm to about 2 nm. Nanopores useful in the present disclosure include any pore capable of permitting the linear translocation of the nucleic acid molecule from one side to the other at a velocity amenable to monitoring techniques, such as techniques to detect current fluctuations.

In some embodiments, the nanopore is disposed within a membrane, thin film, layer, or bilayer. For example, biological (e.g., proteinaceous) nanopores can be inserted into an amphiphilic layer such as a biological membrane, for example a lipid bilayer. An amphiphilic layer is a layer formed from amphiphilic molecules, such as phospholipids, which have both hydrophilic and lipophilic properties. The amphiphilic layer may be a monolayer or a bilayer. The amphiphilic layer may be a co-block polymer. Alternatively, a biological pore may be inserted into a solid state layer.

In some embodiments, the membrane, thin film, layer, or bilayer separates a first aqueous partition (e.g., a conductive medium) and a second aqueous partition (e.g., a conductive medium) to provide a nonconductive barrier between the first aqueous partition and the second aqueous partition. The nanopore, thus, provides communication (e.g., fluid communication) between the first and second aqueous partitions. In some embodiments, the pore provides the only communication (e.g., fluid communication) between the first and second aqueous partitions. In some embodiments, the aqueous partitions comprise electrolytes or ions that can flow from the first aqueous partition into the second aqueous partition through the interior of the nanopore. Any suitable conductive medium can be used, such as, without limitation, a buffer solution. The conductive medium of the first and second aqueous partition may be the same or different, and either one or both may comprise one or conductive medium of the first and second aqueous partition described herein comprises a viscosity-altering substance or a velocity-altering substance. In some embodiments, the membrane comprises at least, or at least about 20, 50, 100, 200, 500, 750, 1,000, 1,250, 1,500, 1,750, 2,000, 2,500, 3,000, 4,000, 5,000, or more, or a number in a range defined any two of the preceding values (e.g., 20-5,000, 100-4,000, 1,500-2,500, 1,750-3,000, etc.) nanopores.

In some embodiments, nanopores can be biological pores or solid state pores. In some embodiments, the nanopore comprises a protein, such as alpha hemolysin, anthrax toxin and leukocidins, and outer membrane proteins/porins of bacteria such as Mycobacterium smegmatis porins (Msp), including MspA, outer membrane porins such as OmpF, OmpG, OmpATb, and the like, outer membrane phospholipase A and Neisseria autotransporter lipoprotein (NalP), and lysenin. In some embodiments, a nanopore includes alpha-helix bundle pores comprising a barrel or channel that is formed from α-helices. Suitable α-helix bundle pores include, but are not limited to, inner membrane proteins and an outer membrane proteins, such as WZA and ClyA toxin. In some embodiments, a nanopore is a homolog or derivative of any nanopore provided herein. A “homolog,” as used herein, is a gene or protein from another species that has a similar structure and evolutionary origin. By way of an example, homologs of wild-type MspA, such as MppA, PorMl, PorM2, and Mmcs4296, can serve as the nanopore in the present disclosure. In some embodiments, protein nanopores self-assemble and are essentially identical to one another. In some embodiments, the nanopore is a genetically engineered protein nanopore, or a “derivative” of a nanopore. In some embodiments, a derivative of a nanopore has amino acid substitutions to alter charge, e.g., from the creation of a fusion protein (e.g., an enzyme+alpha-hemolysin). In some embodiments, the protein nanopores is wild-type or is modified to contain at least one amino acid substitution, deletion, or addition. In some embodiments, the nanopores includes DNA-based structures, such as generated by DNA origami techniques. In some embodiments, the nanopore is an MspA or homolog or derivative thereof. MspA can be formed from multiple monomers. In some embodiments, the pore is homomonomeric or heteromonomeric, where one or more of the monomers contains a modification or difference from the others in the assembled nanopore.

In some embodiments, the adaptive nucleic acid conduit includes additional components to facilitate or regulate the translocation of the nucleic acid through the nanopore. In some embodiments, the adaptive nucleic acid conduit comprises a helicase or a polymerase associated with the nanopore. In some embodiments, the component is a molecular brake, which is a moiety that regulates the rate of translocation of the nucleic acid through the nanopore. In some embodiments, adaptive nucleic acid conduit includes a translocase, a polymerase, a helicase, an exonuclease, or topoisomerase. In some embodiments, the adaptive nucleic acid conduit includes exonucleases, which can include exonuclease I, exonuclease III, lambda exonuclease, or a variant or homolog thereof. For any aspect herein, homologs, derivatives, and other variant proteins, as described herein, can preferably be at least 50% homologous to the reference protein based on amino acid sequence identity. More preferably, the variant polypeptide may be at least 55%, at least 60%, at least 65%, at least 70%, at least 75%, at least 80%, at least 85%, at least 90% and more preferably at least 95%, 97%, 98%, or 99% homologous based on amino acid identity to the reference protein. Homology can be determined by any suitable option. It is noted that homologs or variants can possess sequence and structural modifications so long as they retain the proper functionality for translocation. While exonucleases often contain enzymatic functions for excising portions of the nucleic acids, such enzymes can be modified to ablate such nuclease function while preserving the ability to bind and translocate the nucleic acid polymer. In some embodiments, the adaptive nucleic acid conduit includes an Hel308 helicase, a RecD helicase, a Tral helicase, a Tral subgroup helicase, an XPD helicases, or a variant or homolog thereof. In some embodiments, the adaptive nucleic acid conduit includes DNA polymerases such as phi29 DNA polymerase (sometimes referred to as phi29 DNAP), Klenow fragment, or a variant or homolog thereof. In some embodiments, the adaptive nucleic acid conduit includes topoisomerases that can include a gyrase, or a variant or homolog thereof.

### Nucleic Acids

In accordance with the methods, systems and kits of some embodiments described herein, the first aqueous partition includes nucleic acid molecules that collectively encode at least 2, 3, 4, 5, 6, 7, 8, 9, 10, 15, 20, 25, 30, 35, 40, 50, 100, 200, 300, 400, 500, 600, 700, 800, 900, 1,000, or more, or a number in a range defined by any two of the preceding values (e.g., 2-1,000, 5-500, 10-300, 20-700, etc.) different polypeptides. In some embodiments, the first aqueous partition include mono-cistronic nucleic acid molecules. In some embodiments, the first aqueous partition include poly-cistronic nucleic acid molecules (e.g., the nucleic acid molecule encodes 2, 3, 4, 5, 6 or more polypeptides in a single strand). In some embodiments, the first aqueous partition includes nucleic acid molecules that collectively encode at least 2, 3, 4, 5, 6, 7, 8, 9, 10, 15, 20, 25, 30, 35, 40, 50, 100, 200, 300, 400, 500, 600, 700, 800, 900, 1,000, or more, or a number in a range defined by any two of the preceding values (e.g., 2-1,000, 5-500, 10-300, 20-700, etc.) different polypeptides that correspond to a polypeptide member of the specified mixture. In some embodiments, the plurality of nucleic acids encode at least one, 2, 3, 4, 5, 6, 7, 8, 9, 10, 15, 20, 25, 30, 35, 40, 50, 100, 200, 300, 400, 500, 600, 700, 900, 1,000, or more, or a number in a range defined by any two of the preceding values (e.g., 1-1,000, 5-500, 10-300, 20-700, 2-50, etc.) different polypeptides that do not correspond to a polypeptide member of the specified mixture.

In accordance with the methods, systems and kits of some embodiments described herein, any suitable nucleic acids that encode polypeptides can be used. In some embodiments, in addition to sequences encoding a polypeptide, a nucleic acid includes one or more regulatory sequences. Generally, translation initiation for a particular transcript is regulated by particular sequences at or 5′ of the 5′ end of the coding sequence of a transcript. For example, a coding sequence can begin with a start codon configured to pair with an initiator tRNA. While naturally-occurring translation systems typically use Met (AUG) as a start codon, it will be readily appreciated that an initiator tRNA can be engineered to bind to any desired triplet or triplets, and accordingly, triplets other than AUG can also function as start codons in certain embodiments. Additionally, sequences near the start codon can facilitate ribosomal assembly, for example a Kozak sequence ((gcc)gccRccAUGG, SEQ ID NO: 542, in which R represents “A” or “G”) or Internal Ribosome Entry Site (IRES) in typical eukaryotic translational systems, or a Shine-Delgarno sequence (GGAGGU, SEQ ID NO: 543) in typical prokaryotic translation systems. As such in some embodiments, a transcript comprising a “coding” polynucleotide sequence, for example a bacteriocin polynucleotide, comprises an appropriate start codon and translational initiation sequence. In some embodiments, for example if two or more “coding” polynucleotide sequences are positioned in cis on a transcript, each polynucleotide sequence comprises an appropriate start codon and translational initiation sequence(s). In some embodiments, for example if two or more “coding” polynucleotide sequences are positioned in cis on a transcript, the two sequences are under control of a single translation initiation sequence, and either provide a single polypeptide that can function with both encoded polypeptides in cis. For nucleic acids that are transcribed, any suitable regulatory sequences for transcription can be included. In some embodiments, a promoter drives expression of (e.g., is operably linked to) nucleic acids encoding a polypeptide member as described herein. Any suitable promoter can be selected, and placed in cis with a nucleic acid sequence to be expressed. For example, a nucleic acid molecule can include a T7 promoter (SEQ ID NO: 669) operably linked to the nucleic acid sequence encoding the polypeptide member. Suitable promoters include, without limitation, any one of the sequences set forth in SEQ ID NOs: 544-698.

In accordance with the methods, systems and kits of some embodiments described herein, each of the plurality of nucleic acids comprises a barcode that identifies the polypeptide encoded by the nucleic acid molecule, wherein the adaptive nucleic acid conduit is configured to selectively translocate the nucleic acid molecule into the second aqueous partition based on the detected sequence of the barcode. The barcode can be at the 5′ or 3′ end of the nucleic acid, depending on the direction in which the nucleic acid molecule translocates through the nanopore, and may be positioned to enter the nanopore before the sequence encoding the polypeptide.

In accordance with the methods, systems and kits of some embodiments described herein, the plurality of nucleic acids comprises nucleic acids encoding polypeptides of one or more functional classes. In some embodiments, the plurality of nucleic acids comprises a library of nucleic acids encoding different polypeptides of a functional class. In some embodiments, the functional class includes, without limitation, antimicrobial peptides, bacteriocins, cytokines, peptide toxins, signaling molecules, hormones, etc.). In some embodiments, the plurality of nucleic acids encodes a plurality of different antimicrobial peptides and/or a plurality of different bacteriocins. In some embodiments, the plurality of nucleic acids encodes a plurality of different bacteriocins.

### Nucleic Acid Expression Solutions

Systems, methods, and kits of some embodiments include a nucleic acid expression solution comprising at least a translation solution. In some embodiments, the nucleic acid expression solution includes a transcription solution. In some embodiments, the nucleic acid expression solution includes a transcription and translation solution. For example and without limitation, the accepted nucleic acid molecules can be expressed first in a nucleic acid expression solution that includes a transcription solution to generate RNA, then the generated RNA can be transferred to another nucleic acid expression solution that includes a translation solution to generate the polypeptides encoded by the nucleic acid molecules. In some embodiments, the plurality of nucleic acids comprises RNA. In some embodiments, plurality of nucleic acids comprise RNA, and the nucleic acid expression solution comprises a translation solution. In some embodiments, the nucleic acids comprise DNA, and nucleic acid expression solution comprises a transcription and translation solution. In some embodiments, the plurality of nucleic acids comprises double-stranded DNA. In systems, methods, and kits of some embodiments, the accepted nucleic acid molecules are transcribed in the nucleic acid expression solution.

Translation solutions can be useful for translating nucleic acids in accordance with the methods, systems and kits of some embodiments described herein. Suitable translation solutions can comprise, consist essentially of, or consist of reagents for in vitro translation (which, for convenience, may be referred to herein as “translation reagents”), and as such can be configured for in vitro translation of a transcript such as an RNA. In some embodiments, a translation solution is comprised by a translation station of a microfluidic system as describe herein. In some embodiments, the translation solution further comprises a transcription solution comprising reagents for transcription (which, for convenience, may be referred to herein as “transcription reagents”), and thus is configured for in vitro transcription and translation, for example to transcribe and translate a candidate nucleic acid encoding a candidate antimicrobial peptide as described herein. It is contemplated that in vitro transcription and translation in a single solution (such as a transcription solution further comprising a translation solution as described herein) can facilitate efficient in vitro production of accepted nucleic acid molecules in accordance with methods, systems, and kits of some embodiments.

In accordance with the methods, systems and kits of some embodiments described herein, the translation solution comprises, consists essentially of, or consists of one or more translation reagents Examples of translation reagents include a ribosome, a buffer, an amino acid, a tRNA (which may be conjugated to an amino acid), a lysate or extract such as an E. coli lysate or E. coli extract, and a cofactor or metallic ion such as Mg2+, or a combination of two or more of any of the listed items. In accordance with the methods, systems and kits of some embodiments described herein the translation solution further comprises a transcription solution, and thus is configured for in vitro transcription and translation. As described herein, a transcription solution further comprising a translation solution contemplates a single solution that is suitable for in vitro transcription and translation. As such, a transcription solution further comprising a translation solution encompasses a single transcription/translation solution, and well as translation solution with discrete sub-environments, at least some of which are suitable for transcription. It will be appreciated that some components of a transcription and/or translation solution, for example ribosomes, may not be liquids, and could potentially be isolated from the transcription and/or translation solution, for example by filtration and/or centrifugation. Nucleic acid expression solutions of methods, systems and kits of some embodiments described herein (and which can be comprised by translation solutions as described herein) can comprise, consist essentially or, or consist of one or more transcription reagents. Examples of transcription reagents include an RNA polymerase, a buffer, a nucleic acid mix (for example, NTPs including ATP, GTP, CTP, and UTP), a cofactor or metallic ion such as Mg2+, a transcription inducer (such as a transcription factor, IPTG, or lactose), a polyadenylation enzyme, a capping enzyme, a lysate or extract such as a bacterial lysate or extract such as an E. coli lysate or E. coli extract, an SP6 polymerase, a T3 polymerase, a T7 RNA polymerase, or a mixture of two or more of any of the listed items. The transcription solution can be useful for transcribing a template, such as a candidate nucleic acid as described herein. Translation solutions of methods, kits, and systems of some embodiments include one or more transcription reagents in combination with one or more translation reagents.

In some embodiments, the translation solution comprises a post-translational modification enzyme. Examples of post-translational modification enzymes include, but are not limited to a cleavage enzyme, a kinase, a phosphatase, a glycosyltransferase, or a mixture of any two of the listed items.

In some embodiments, a microfluidic device of the present disclosure includes a translation station, which may be the same as the second aqueous partition, or may be in fluid communication with the second aqueous partition. In some embodiments, the translation station comprises the translation solution. In some embodiments, the microfluidic device comprises a transcription station and a transcription station, which may be the same station, or may be different stations. For example, in some embodiments, the transcription station comprises a single transcription/translation station configured for in vitro transcription and translation of a nucleic acid. In some embodiments, the translation station is configured to perform in vitro translation. In some embodiments, the transcription station is configured to perform in vitro transcription. In some embodiments, the translation station further comprises the transcription station (for example as a single environment, or as two discrete environments), and is configured to perform in vitro transcription and translation. In some embodiments, the microfluidic device comprises a transcription station comprising the transcription solution, and a separate translation station comprising the translation solution.

In some embodiments, the translation station is configured to receive a translation solution and/or one or more translation reagents and/or transcription reagents as described herein. In some embodiments, the transcription station is in fluid communication with one or more reservoirs comprising transcription reagents and/or translation reagents. As such, in some embodiments, a translation station initially does not include a translation solution, but is configured to receive a translation solution, or one or more reagents.

In accordance with the methods, systems and kits of some embodiments described herein, the translation solution is configured to receive the accepted nucleic acid molecules.

It is contemplated that immobilizing accepted nucleic acid molecules on a substrate such as a bead can be useful for manipulation of accepted nucleic acid molecules and its corresponding polypeptides in a microfluidic environment, for example in a microdroplet. In accordance with the methods, systems and kits of some embodiments described herein, the translation solution comprises a substrate. Examples of suitable substrates include a bead, a nanoparticle, a well, a membrane, nitrocellulose, PVDF, nylon, an acetate derivative, a matrix, a pore, plastic, metal, glass, a polymer, a polysaccharide, and a paramagnetic compound, or a combination of two or more of any of the listed items. In some embodiments, the candidate nucleic acid is immobilized on the substrate.

In accordance with the methods, systems and kits of some embodiments described herein, the translation solution is at a microliter-scale. For example, the translation solution may have a volume of 1 μl-1000 μl, 1 μl-50 μl, 1 μl-500 μl, 1 μl-900 μl, 50 μl 100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl.

### Polypeptides

In accordance with the methods, systems and kits of some embodiments described herein, polypeptides encoded by the nucleic acids and/or in the specified mixture of polypeptides can include any suitable polypeptides. In some embodiments, polypeptides include antimicrobial peptides, bacteriocins, and signal molecules.

In accordance with the methods, systems and kits of some embodiments described herein, the specified mixture comprises two different polypeptide members at a ratio or stoichiometry (e.g., a first to second polypeptide member, first to third, second to third, or third to fourth, or fourth to fifth, etc.) of 1:2, 1:3, 1:4, 1:5, 1:6, 1:7, 1:8, 1:9, 1:10, 1:20, 1:30, 1:40, 1:50, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, 9:10, 19:20, 28:30, 29:30, 39:40, or 49:50, or a ratio in a range defined by any two of the preceding values (e.g., about 1:50 to 49:50, about 1:10 to 9:10, about 1:2 to about 4:5, about 1:5 to about 2:3, etc.). In some embodiments, the specified mixture comprises a first polypeptide member and a second (different) polypeptide member at a ratio of the first polypeptide member to the second polypeptide member of 1:2, 1:3, 1:4, 1:5, 1:6, 1:7, 1:8, 1:9, 1:10, 1:20, 1:30, 1:40, 1:50, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, 9:10, 19:20, 28:30, 29:30, 39:40, or 49:50, or a ratio in a range defined by any two of the preceding values (e.g., about 1:50 to 49:50, about 1:10 to 9:10, about 1:2 to about 4:5, about 1:5 to about 2:3, etc.). In some embodiments, the specified mixture comprises at least two different polypeptide members at a ratio other than 1:1. It is noted that different pairs of polypeptide members in the specified mixture can have different ratios to each other. Therefore, it is contemplated that the ratios of three or more polypeptide members to each other can be ascertained by the individual (pair-wise) ratios of the polypeptide members to each other. For example, a first and second polypeptide members can have a ratio of 1:2, and a second and third polypeptide members can have a ratio of 2:5, so that the ratio of the first to the second to the third polypeptide members is 1:2:5, respectively. In some embodiments, the desired ratio comprises a ratio of a first polypeptide member to a second polypeptide member to a third polypeptide member of about 1:1:2, 1:2:2, 1:1:3, 1:2:3, 1:3:3, 2:2:3, or 2:3:3. In some embodiments, the specified mixture of two or more polypeptides is designed such that a composition having the specified mixture exhibits a desired effect. In some embodiments, where the two or more polypeptides include antimicrobial peptides, a composition having the specified mixture targets an undesired microbial organism.

In some embodiments, the specified mixture includes an amount of each of the polypeptide members, independently, at about 1 μM to about 10 μM, about 10 μM to about 100 μM, about 100 μM to about 1 nM, about 1 nM to about 10 nM, about 10 nM to about 100 nM, about 100 nM to about 1 μM, about 1 μM to about 10 μM, about 10 μM to about 100 μM, about 100 μM to about 1 mM, or about 1 mM to about 10 mM.

### Bacteriocins and Antimicrobial Peptides

As used herein, “bacteriocin,” and variations of this root term, has its customary and ordinary meaning as understood by one of skill in the art in view of this disclosure. It refers to a polypeptide that can neutralize at least one microbial organism. “Bacteriocin” also encompasses a cell-free or chemically synthesized version of such a polypeptide, for example an engineered bacteriocin in accordance with some embodiments herein. A bacteriocin can exert cytotoxic or growth-inhibiting effects on one or a plurality of other microbial organisms. Non-limiting examples of bacteriocins are set forth in the even numbered sequences of SEQ ID NOS: 4-450 and the odd numbered sequences of SEQ ID NOS: 699-737. Non-limiting examples of nucleic acids encoding these bacteriocins are provided as odd numbered sequences of SEQ ID NOs: 5-451 and even numbered sequences of 700-738. Detailed descriptions of bacteriocins and some polynucleotide sequences that encode bacteriocins, including methods and compositions for using bacteriocins to control the growth of microbial cells can be found, for example, in U.S. Pat. No. 9,333,227, which is hereby incorporated by reference in its entirety. Some examples of suitable bacteriocins and categories of bacteriocins are taught in Tables 1.1 and 1.2 of U.S. Pat. No. 9,333,227. It is contemplated that any of these bacteriocins can be subject to further engineering. For example, variants and/or modifications of these bacteriocins can be encoded in the nucleic acids or be part of a specified mixture in accordance with some embodiments herein. In some embodiments, a bacteriocin has at least about 50% identity, for example, at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the polypeptides set forth in the even numbered sequences of SEQ ID NOS: 4-450 and the odd numbered sequences of SEQ ID NOS: 699-737, including ranges between any two of the listed values, for example 70%-100%, 75%-100%, 80%-100%, 85%-100%, 90%-100%, 95%-100%, 97%-100%, 99%-100%, 70%-99%, 75%-99%, 80%-99%, 85%-99%, 90%-99%, 95%-99%, 97%-99%, 70%-95%, 75%-95%, 80%-95%, 85%-95%, 90%-95%, 70%-90%, 75%-90%, 80%-90%, and 85%-90%. In some embodiments, the bacteriocin is a circularized bacteriocin.

Antimicrobial peptides are a class of peptides that kill or arrest the growth of microbial organisms. As used herein “antimicrobial peptide” (including variations of this root term) has its customary and ordinary meaning as would be understood by one of ordinary skill in the art in view of this disclosure. Classically, antimicrobial peptides have been described as peptides produced by the innate immune systems of invertebrates and vertebrates. Thus, while bacteriocins have classically been referred to a class of microbial gene products that target microbial organisms, antimicrobial peptides have classically been referred to as a class of invertebrate and vertebrate gene products that target microbial organisms.

Examples of classical antimicrobial peptides suitable for methods, systems, and kits of some embodiments herein are known in the art, and can be found, for example, at The Antimicrobial Peptide Database accessible on the world wide web at aps.unmc.edu/AP/, which is incorporated herein by reference in its entirety. Over 1000 antimicrobial peptides and variants thereof have been identified and cataloged. The Antimicrobial Peptide Database is described in Wang et al. (2016), Nucleic Acids Res. 44(Database issue): D1087-D1093, which is incorporated herein by reference in its entirety. Examples of antimicrobial peptides include antibacterial, antiviral, anti-HIV, antifungal, antiparasitic and anticancer peptides, such as Dermaseptin-B2, Abaecin, Ct-AMP1, Andropin, Aurein 1.1, Lactoferricin B, and Heliomicin. Methods, systems, and kits of some embodiments comprise naturally-occurring antimicrobial peptides, or a nucleic acid encoding the same. Methods, systems, and kits of some embodiments comprise non-naturally occurring antimicrobial peptides, or nucleic acids encoding the same. Methods, systems, and kits of some embodiments include antimicrobial peptides that comprise a mutation or variation in a naturally-occurring antimicrobial peptides, or a nucleic acid encoding the same. Methods, systems, and kits of some embodiments comprise antimicrobial peptides comprising, consisting essentially of, or consisting of non-naturally occurring peptide sequences, or nucleic acids encoding the same.

In accordance with the methods, systems and kits of some embodiments described herein, an antimicrobial peptide is engineered. In some embodiments, the engineered antimicrobial peptide is engineered to have a modified activity or ability to kill or affect the growth of a microbial organism.

Some antimicrobial peptides and/or bacteriocins have cytotoxic activity (e.g. “bacteriocide” effects), and thus can kill microbial organisms, for example bacteria, yeast, algae, synthetic microorganisms, and the like. Some antimicrobial peptides and/or bacteriocins can inhibit the reproduction of microbial organisms (e.g. “bacteriostatic” effects), for example bacteria, yeast, algae, synthetic microorganisms, and the like, for example by arresting the cell cycle.

While many the bacteriocins are naturally-occurring (for example, naturally occurring bacteriocins set forth in the even numbered sequences of SEQ ID NOS: 4-450 and the odd numbered sequences of SEQ ID NOS: 699-737), the skilled artisan will appreciate that in some embodiments of the methods, systems and kits described herein, a bacteriocin comprises a naturally-occurring bacteriocin other than the bacteriocins and encoding nucleotide sequences of the even numbered sequences of SEQ ID NOS: 4-450 and the odd numbered sequences of SEQ ID NOS: 699-737, or a non-naturally-occurring bacteriocin or a synthetic bacteriocin, or a variant thereof. In some embodiments, the antimicrobial peptide (or bacteriocin) does not comprise a lantibiotic.

Several motifs have been recognized as characteristic of bacteriocins. For example, the motif YGXGV (SEQ ID NO: 2), wherein X is any amino acid residue, is an N-terminal consensus sequence characteristic of a class Ila bacteriocin. Accordingly, in some embodiments, a bacteriocin comprises an N-terminal sequence with at least about 50% identity to SEQ ID NO: 2), for example at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 2). In some embodiments, a bacteriocin comprises a N-terminal sequence comprising SEQ ID NO: 2). Additionally, some class IIb bacteriocins comprise a GxxxG motif. Without being limited by any particular theory, it is believed that the GxxxG motif can mediate association between helical proteins in the cell membrane, for example to facilitate bacteriocin-mediated neutralization through cell membrane interactions. As such, in some embodiments, the bacteriocin comprises a motif that facilitates interactions with the cell membrane. In some embodiments, the bacteriocin comprises a GxxxG motif. Optionally, the bacteriocin comprising a GxxxG motif can comprise a helical structure. In addition to structures described herein, “bacteriocin” as used herein also encompasses structures that have substantially the same effect on microbial cells as any of the bacteriocins explicitly provided herein.

It is contemplated herein that an antimicrobial peptide or a bacteriocin can comprise a fusion of two or more polypeptides, for example two or more polypeptides having antimicrobial or bacteriocin activity. In some embodiments an antimicrobial peptide or a bacteriocin comprises a chimeric protein.

In some embodiments of the methods, systems and kits described herein, a candidate antimicrobial peptide or a bacteriocin inhibits the growth and/or reproduction of a microbial organism. Inhibition of growth or reproduction has its customary and ordinary meaning as understood by one of skill in the art in view of this disclosure. It refers to a decrease in or arrest of proliferation of microbial organisms (or a decrease in the rate of proliferation of microbial organisms), for example, arrest of the cell cycle and/or killing of microbial organisms. In accordance with methods, systems, and kits of some embodiments herein, an inhibition of growth and/or reproduction of a microbial organism represents a quantity of microbial organisms, and/or a rate of growth of the microbial organisms that does not exceed a reference level. In some embodiments of the methods, systems and kits described herein, inhibition of growth comprises a quantity of a microbial organism remaining constant or decreasing over time. The decrease can be compared to a reference level from an earlier point in time.

In some embodiments, inhibition of growth comprises a decrease in the size or amount of the microbial organism. In some embodiments, inhibition of growth comprises a decrease in an organelle of the microbial organism, for example a chloroplast or mitochondrion. In some embodiments, inhibition of growth comprises killing the microbial organism, for example through lysis, apoptosis, and/or necrosis. In some embodiments, inhibition of reproduction of a microbial organism comprises a decrease or a cessation in the rate of cell division or cell doubling. In some embodiments, inhibition of reproduction of a microbial organism comprises a decrease or a cessation in an increase in an amount of the microbial organism.

In some embodiments, a particular neutralizing activity or ranges of activities (e.g. cytotoxicity or arrest of microbial reproduction) is selected based on the type of microbial regulation that is desired and the particular strains or species of microbial organisms being targeted. As such, in some embodiments, particular bacteriocins (and/or antimicrobial peptides) or combinations of bacteriocins (and/or antimicrobial peptides) are selected. In some embodiments, for example if contaminating cells are to be killed, at least one cytotoxic bacteriocin is provided. In some embodiments, a bacteriocin or combination of bacteriocins (and/or antimicrobial peptides) which is effective against contaminants which commonly occur in a particular culture, or a particular geographic location, or a particular type of culture grown in a particular geographic location are selected. Without being limited by any particular theory, many bacteriocins can have neutralizing activity against microbial organisms that typically occupy the same ecological niche as the species that produces the bacteriocin. As such, in some embodiments, when a particular spectrum of bacteriocin activity is desired, a bacteriocin is selected from a host species that occupies the same (or similar) ecological niche as the microbial organism or organisms targeted by the bacteriocin. In some embodiments, a particular mixture and/or ratio is selected to target a single microbial organism (which can include targeting one or more than one microbial organisms of that type, for example clonally related microbial organisms). For example a particular type of microbial organism may be targeted more efficiently by a specified mixture and/or ratio of bacteriocins than by a single bacteriocin.

In some embodiments, bacteriocins (and ratios thereof) may be selected based on their ability to neutralize one or more invading organisms which are likely to attempt to grow in a particular culture. In some embodiments, bacteriocins (and ratios thereof) may be selected based on their ability to limit the growth of particular useful microbial strains in an environment, for example in an industrial feedstock, or in a fermenter, or in a food, pharmaceutical, or cosmetic manufacturing environment, or in a tissue environment such as a gut or skin microbiome, or in maintaining or tuning a microbial population in a plant, a plant root, and/or soil, or in preserving or maintaining the quality of a food, drug or cosmetic product. In some embodiments, one or more bacteriocin activities (and/or ratios) are selected based on one or more microbial strains or a population of microbial strains an existing environment. For example, in some embodiments, if particular invaders are identified in an environment, a panel of neutralizing bacteriocins (and ratios thereof) can be selected to neutralize the identified invaders. In some embodiments, the bacteriocins are selected to neutralize all or substantially all of the microbial cells in an environment, for example to eliminate an industrial culture in a culture environment so that a new industrial culture can be introduced to the culture environment, or to prevent or inhibit contamination of a pharmaceutical or cosmetic manufacturing environment, or to prevent or minimize contamination or spoilage of a food, drug, or cosmetic product.

For example and without limitation, in some embodiments, an anti-fungal activity (such as anti-yeast activity) is desired. A number of bacteriocins with anti-fungal activity have been identified. For example, bacteriocins from Bacillus can have neutralizing activity against yeast strains (see Adetunji and Olaoye (2013) Malaysian Journal of Microbiology 9:130-13, hereby incorporated by reference in its entirety), an Enterococcus faecalis peptide (WLPPAGLLGRCGRWFRPWLLWLQ SGAQY KWLGNLFGLGPK, SEQ ID NO: 1) can have neutralizing activity against Candida species (see Shekh and Roy (2012) BMC Microbiology 12:132, hereby incorporated by reference in its entirety), and bacteriocins from Pseudomonas can have neutralizing activity against fungi such as Curvularia lunata, Fusarium species, Helminthosporium species, and Biopolaris species (Shalani and Srivastava (2008) The Internet Journal of Microbiology. Volume 5 Number 2. DOI: 10.5580/27dd-accessible on the worldwide web at archive.ispub.com/journal/the-internet-journal-of-microbiology/volume-5-number-2/screening-for-antifungal-activity-of-pseudomonas-fluorescens-against-phytopathogenic-fungi.html #sthash.d0Ys03UO.1DKuTIUS.dpuf, hereby incorporated by reference in its entirety). By way of example, botrycidin AJ1316 (see Zuber, P et al. (1993) Peptide Antibiotics. In Bacillus subtilis and Other Gram-Positive Bacteria: Biochemistry, Physiology, and Molecular Genetics ed Sonenshein et al., pp. 897-916, American Society for Microbiology, hereby incorporated by reference in its entirety) and alirin B1 (see Shenin et al. (1995) Antibiot Khimioter 50:3-7, hereby incorporated by reference in its entirety) from B. subtilis can have antifungal activities. As such, in some embodiments, for example embodiments in which neutralization of a fungal microbial organism is desired, a bacteriocin comprises at least one of botrycidin AJ1316 or alirin B1.

For example, in some embodiments, bacteriocin activity in a culture of a particular microorganism (or collection of different microorganisms) is desirable, and bacteriocins are selected in specified ratios in order to neutralize microorganisms other than the desired microorganism(s). For example, in some embodiments, bacteriocins are selected in particular ratios in order to neutralize invading microbial organisms typically found in a cyanobacteria culture environment, while preserving the cyanobacteria. Clusters of conserved bacteriocin polypeptides have been identified in a wide variety of cyanobacteria species. For example, at least 145 putative bacteriocin gene clusters have been identified in at least 43 cyanobacteria species, as reported in Wang et al. (2011), Genome Mining Demonstrates the Widespread Occurrence of Gene Clusters Encoding Bacteriocins in Cyanobacteria. PLOS ONE 6(7): e22384, hereby incorporated by reference in its entirety. Exemplary cyanobacteria bacteriocins are set forth in SEQ ID NOs: 420, 422, 424, 426, 428, 430, 432, 434, 436, 438, 440, 442, 444, 446, 448, and 450.

In some embodiments, a specified composition includes a desired ratio of bacteriocins selected to target an undesired microbial organism or population of undesired microbial organisms. For example, if an environment such as a culture medium, feedstock, fermenter, bioreactor, or microbiome contains, or is at risk of containing a population of undesired microbial organisms, a ratio of bacteriocins can be selected to target those undesired microbial organisms. In some embodiments, the desired ratio of bacteriocins is selected to balance a population of a microbiome of an animal (for example a horse, cow, sheep, pig, donkey, dog, cat, or non-human primate), a human organ (e.g., skin or a gut), or a plant root and/or soil microbiome, or to preserve a product such as a food product (human or non-human animal), pharmaceutical, or cosmetic product.

### Signal Molecules

In some embodiments, polypeptides comprise one or more signal molecules. As used herein, a “signal molecule” has its customary and ordinary meaning as understood by one of skill in the art in view of this disclosure. It refers to a secreted molecule that is capable of modulating, inducing, or inhibiting an activity or process in the cell that produced it, or in a different cell (a subject cell can be a microbial cell or a non-microbial cell, for example a cell of a multicellular organism such as an animal or plant). Example signal molecules include, but are not limited to, signaling peptides, quorum sensing molecules (for example, quorum sensing peptides), signal transduction receptor ligands, growth factors, hormones, and cytokines. In some embodiments, a signal molecule comprises, consists essentially of, or consists of quorum sensing molecules (for example, quorum sensing peptides), signal transduction receptor ligands, growth factors, hormones, or cytokines. In some embodiments, a signal molecule comprises, consists essentially of, or consists of a combination of two or more of quorum sensing molecules (for example, quorum sensing peptides), signal transduction receptor ligands, growth factors, hormones, and cytokines, which can include combinations of two or more of the same type of molecule (for example a combination of two signaling peptides or a combination of two receptor ligands), as well as combinations of two different kinds of molecules (e.g., a combination of a cytokine and a hormone). In some embodiments, the signal molecule is for microbe-host dialog, and as such, the signal molecule is selected to target one or more cells of a host organism, for example a plant or animal. In some embodiments, the signal molecule stimulates, inhibits, increases, or decreases the production of bacteriocins and/or the growth rate of a subpopulation of a flora. In some embodiments, the signal molecule comprises, consists of, or consists essentially of a quorum sensing peptide, or a variant thereof as described herein.

Example quorum sensing peptides suitable for methods, systems, kits and/or encoded by nucleic acids of some embodiments include, but are not limited to, quorum sensing peptides. Without being limited by theory, it is contemplated that microbial cells, such as gram-positive bacteria use quorum sensing peptides to orchestrate cell-to-cell communication. A review of quorum sensing peptides can be found in Rajput et al., PLOS One DOI: 10.1371/journal.pone.0120066 Mar. 17, 2015, pp. 1-16, which is hereby incorporated by reference in its entirety. The quorum sensing peptides can induce activation of downstream response regulators and/or transcription factors in a target microbial cell.

In some embodiments, the quorum sensing peptides are naturally-occurring. In some embodiments, the quorum sensing peptide comprises, consists essentially of, or consists of a variant of a naturally-occurring quorum sensing peptide. In some embodiments, the quorum sensing peptide comprises, consists essentially of, or consists of a synthetic peptide. Information on quorum sensing peptides, including example sequences, can be found on the quorumpeps database, accessible on the world wide web at quorumpeps.ugent.be., which is hereby incorporated by reference in its entirety.

Cytokines are a class of signal molecules that are typically produced by cells, such as cells of the immune system, and capable of inducing a response in other cells. A number of different cytokines can be used in methods, systems, kits and/or encoded by nucleic acids of some embodiments herein. It is contemplated that a composition comprising a bacteriocin and a cytokine in accordance with some embodiments can be useful to induce antimicrobial activity (by the bacteriocin(s)), and a host response, for example immune cell suppression or immune cell stimulation (by the cytokine(s)). In some embodiments, the cytokine comprises, consists essentially of, or consists of a naturally-occurring cytokine, variant of a naturally occurring, or synthetic cytokine. A number of suitable cytokines can be used in methods, systems, and kits in accordance with some embodiments herein, including, but not limited to IL-1, IL-2, IL-3, IL-4, IL-5, IL-6, IL-7, IL-8, IL-9, IL-10, IL-11, IL-12, IL-13, IL-15, IL-16, IL-17, IL-18, IL-19, IL-20, IL-21, IL-22, IL-23, IFN-α, IFN-β, IFN-γ, TNF-α, TNF-β, TGF-β1, M-CSF, G-CSF, and GM-CSF, variants of any of these, or any combination of two or more of these.

Hormones are a class of signal molecules that are typically produced by cells of multicellular organisms, and signal to other cells, frequently circulating through different tissues and/or organs of a multicellular organism. A number of different hormones can be used in the methods, systems, and kits of some embodiments herein, and/or be encoded by nucleic acids of some embodiments herein. It is contemplated that a composition comprising a bacteriocin and a hormone in accordance with some embodiments can be useful to induce antimicrobial activity (by the bacteriocin(s)), along with a host response, for example cell growth or proliferation (by the hormone(s)). In some embodiments, the cytokine comprises, consists essentially of, or consists of a naturally-occurring hormone, variant of a naturally occurring hormone, or synthetic hormone. Example hormones suitable for methods, systems, kits and/or encoded by nucleic acids of some embodiments include, but are not limited to, protein and peptide hormones, for example activin and inhibin, adiponectin, adipose-derived hormones, adrenocorticotropic hormone, agouti gene, agouti signaling peptide, allatostatin, amylin, amylin family, angiotensin, ANGPTL8, asprosin, atrial natriuretic peptide, big gastrin, bovine somatotropin, bradykinin, brain-derived neurotrophic factor, calcitonin, ciliary neurotrophic factor, corticotropin-releasing hormone, crustacean neurohormone family, endothelin, enteroglucagon, erythroferrone, fellutamide, FGF15, FGF15/19, FGF19, FNDC5, follicle-stimulating hormone, gastrin, gastroinhibitory peptide, ghrelin, glucagon-like peptide-1, gonadotropin, gonadotropin release inhibitor, gonadotropin-releasing hormone, granulocyte colony-stimulating factor, growth hormone, growth hormone-releasing hormone, hepcidin, human chorionic gonadotropin, human placental lactogen, incretin, insulin, insulin analog, insulin aspart, insulin degludec, insulin glargine, insulin lispro, insulin-like growth factor, insulin-like growth factor 1, insulin-like growth factor 2, leptin, limostatin, liraglutide, little gastrin I, luteinizing hormone, melanocortin, melanocyte-stimulating hormone, alpha-melanocyte-stimulating hormone, beta-melanocyte-stimulating hormone, gamma-melanocyte-stimulating hormone, minigastrin, N-terminal prohormone of brain natriuretic peptide, nerve growth factor, neuropeptide VF precursor, neurotrophin-3, neurotrophin-4, NPH insulin, obestatin, osteocalcin, parathyroid hormone, peptide hormone, peptide YY, plasma renin activity, pramlintide, preprohormone, prolactin, relaxin, relaxin family peptide hormones, renin, salcatonin, sauvagine, secretin, secretin family, sincalide, stanniocalcin, teleost leptins, temporin, thyroid-stimulating hormone, thyrotropin-releasing hormone, urocortin, urocortin II, urocortin III, vasoactive intestinal peptide, vitellogenin, variants of any of these, or any combination of two or more of these.

Detecting Inhibition of Growth and/or Reproduction

Inhibition of growth and/or reproduction, or a lack thereof, of a microbial organism can be detected directly or indirectly via a number of suitable approaches and apparatuses in accordance with methods, systems, and kits of some embodiments herein. By way of example, inhibition of growth or reproduction of one or more microbial organisms can indicate whether a composition of antimicrobial peptides and/or bacteriocins has a suitable activity in accordance with the methods, systems and kits of some embodiments described herein. Detecting inhibition of growth and/or reproduction, or a lack thereof, can be performed by any number of suitable methods, for example as described herein.

In methods, kits, and systems of some embodiments, inhibition of growth and/or reproduction is detected when a quantity, growth rate, or reproduction rate of a microbial organism is less than, or is less than or equal to a predetermined level. The predetermined level can be a reference point. For example, the predetermined level of some embodiments can be a growth rate or quantity of the microbial organism prior to culturing the microbial organism with the antimicrobial peptide or bacteriocin. For example, the predetermined level of some embodiments can be or the growth rate or quantity of a control microbial organism that is cultured in a control solution environment under the selected culture conditions in the absence of the antimicrobial peptide or bacteriocin and/or in the presence of a sham antimicrobial peptide or a sham bacteriocin that is known to be inactive). In some embodiments, the predetermined level of inhibition of growth and/or reproduction of the microbial organism is a greater level of inhibition than that of a reference naturally-occurring antimicrobial peptide in a corresponding control solution environment containing the same microbial organism under the same culture conditions.

In accordance with the methods, systems and kits of some embodiments described herein detecting inhibition of growth and/or reproduction, or a lack thereof, of the microbial organism comprises quantifying the microbial organism in the solution environment. A decrease (or arrest) in a quantity of the microbial organism in the solution environment over a period of time can indicate inhibition of growth and/or reproduction of the microbial organism. Quantifying the microbial organism may be performed by any method known in the art. In some embodiments of the methods, systems and kits described herein, quantifying the microbial organism comprises detecting and/or measuring the light absorbance of a bacterial culture. In some embodiments, the quantity of the microbial organism is detected by measuring an optical density with a spectrophotometer (for example at OD600). In some embodiments, quantifying the microbial organism comprises determining the amount of a microbial marker such as a protein, RNA sequence or DNA sequence. For example, in some embodiments, quantifying the microbial organism comprises performing RNA or DNA sequencing or qPCR. In some embodiments, quantifying the microbial organism comprises optically, chemically, and/or electromagnetically quantifying the marker (for example, by performing an immunoassay, by performing an enzymatic assay, via chromatography, via mass spectrometry, or the like). In some embodiments, quantifying the microbial organism comprises visually detecting the microbial organism. In some embodiments, a detector such as an optical sensor detects inhibition of growth and/or reproduction, or a lack thereof, of a microbial organism as described herein.

### Methods

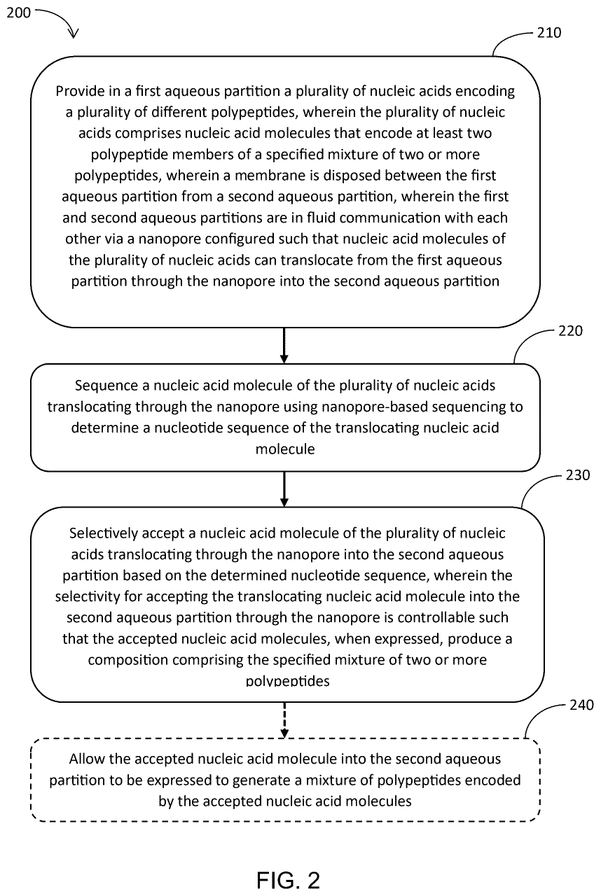

Provided herein are methods of selecting nucleic acids for producing a specified mixture of polypeptides encoded by the nucleic acids, and methods of producing the specified mixture of polypeptides encoded by the nucleic acids. With reference to FIG. 2, the method 200 can include, at block 210, providing in a first aqueous partition a plurality of nucleic acids encoding a plurality of different polypeptides, wherein the plurality of nucleic acids comprises nucleic acid molecules that encode at least two polypeptide members of a specified mixture of two or more polypeptides, wherein a membrane is disposed between the first aqueous partition and a second aqueous partition, wherein the first and second aqueous partitions are in communication (e.g., fluid communication) with each other via a nanopore configured such that nucleic acid molecules of the plurality of nucleic acids can translocate from the first aqueous partition through the nanopore into the second aqueous partition. The method can further include, at block 220, sequencing a nucleic acid molecule of the plurality of nucleic acids translocating through the nanopore using nanopore-based sequencing to determine a nucleotide sequence of the translocating nucleic acid molecule. The method can also include, at block 230, selectively accepting a nucleic acid molecule of the plurality of nucleic acids translocating through the nanopore into the second aqueous partition based on the determined nucleotide sequence, wherein the selectivity for accepting the nucleic acid molecule into the second aqueous partition through the nanopore is controllable such that the accepted nucleic acid molecules, when expressed, produce a composition comprising the specified mixture of two or more polypeptides. Optionally, the method can include, at block 240, allowing the accepted nucleic acid molecule in the second aqueous partition to be expressed to generate a mixture of polypeptides encoded by the accepted nucleic acid molecules.

The accepted nucleic acid molecule can be expressed using any suitable option. In some embodiments, the second aqueous partition comprises or is in fluid communication with a nucleic acid expression solution comprising at least a translation solution, and wherein the method comprises allowing the accepted nucleic acid molecule in the second aqueous partition to contact the nucleic acid expression solution, whereby the accepted nucleic acid molecules in the nucleic acid expression solution are expressed to generate a mixture of polypeptides encoded by the accepted nucleic acid molecules and produce the specified mixture of two or more polypeptides.

In some embodiments, the method includes recombinantly expressing the accepted nucleic acid molecules to generate the mixture of polypeptides encoded by the accepted nucleic acid molecules. In some embodiments, the accepted nucleic acid molecules are expressed in a microbial organism genetically modified to express the accepted nucleic acid molecules. Exemplary microbial organism that can be used in accordance with embodiments herein include, but are not limited to, bacteria, yeast, and algae, for example photosynthetic microalgae. Furthermore, fully synthetic microorganism genomes can be synthesized and transplanted into single microbial cells, to produce synthetic microorganisms capable of continuous self-replication (see Gibson et al. (2010), “Creation of a Bacterial Cell Controlled by a Chemically Synthesized Genome,” Science 329:52-56, hereby incorporated by reference in its entirety). As such, in some embodiments, the microorganism is fully synthetic. A desired combination of genetic elements, including elements that regulate gene expression, and elements encoding gene products (for example bacteriocins, immunity modulators, poison, antidote, and industrially useful molecules) can be assembled on a desired chassis into a partially or fully synthetic microorganism.

In some embodiments, the method includes enriching for and/or increase representation of nucleic acids encoding polypeptide members of the specified mixture of two or more polypeptides using adaptive sampling of the input nucleic acids (e.g., in the first aqueous partition), e.g., in a system of some embodiments herein. In some embodiments, the nucleic acids encoding polypeptide members of the specified mixture in the first aqueous partition are not in the proportion according to the specified mixture.

Any suitable length of the translocating nucleic acid molecule can be sequenced to determine whether the polypeptide encoded by the translocating nucleic acid molecule corresponds to a polypeptide member of the specified mixture. In some embodiments, the minimum length of the translocating nucleic acid molecule that is sequenced depends on the level of sequence diversity among the plurality of nucleic acids in the first aqueous partition. In some embodiments, at least, or at least about 18, 21, 24, 27, 30, 40, 50, 60, 70, 80, 90, 100, 120, 140, 160, 180, or at least 200 nucleotides, or at most, or at most about 300, 250, 200, 150, 100, 80, or at most 60 nucleotides, or a length in a range defined by any two of the preceding values (e.g., about 18-300, 30-250, 60-200, 18-100, etc.) of the translocating nucleic acid molecule is sequenced to determine whether the polypeptide encoded by the translocating nucleic acid molecule corresponds to a polypeptide member of the specified mixture.

In some embodiments, selectively accepting the translocating nucleic acid molecule comprises allowing the nucleic acid molecule to translocate into the second aqueous partition if at least the identity of the polypeptide encoded by the translocating nucleic acid molecule corresponds to a polypeptide member of the specified mixture, while preventing translocation of nucleic acid molecules from the first aqueous partition into the second aqueous partition if the identity of the polypeptide encoded by the translocating nucleic acid molecule is not a polypeptide member of the specified mixture. In some embodiments, selectively accepting the translocating nucleic acid molecule of the plurality of nucleic acids comprises allowing the nucleic acid molecule to translocate into the second aqueous partition if at least the identity of the polypeptide encoded by the translocating nucleic acid molecule corresponds to a polypeptide member that is deficient for the accepted nucleic acid molecules to generate the mixture of polypeptides when compared to the specified mixture of two or more polypeptides.

In some embodiments, selectively accepting the nucleic acid molecule of the plurality of nucleic acids comprises controlling the rates at which nucleic acid molecules encoding each of the polypeptide members of the specified mixture are accepted into the second aqueous partition. In some embodiments, the rate at which nucleic acid molecules encoding polypeptide members of the specified mixture are accepted into the second aqueous partition is measured over a suitable time interval. In some embodiments, the rate is measured over about 0.1-0.5 seconds, about 0.5-1 seconds, about 1-5 seconds, or about 5-10 seconds, or longer.

In some embodiments, the method includes counting the number of nucleic acid molecules that have been accepted into the second aqueous partition within a specified time period or a specified number of events.

In some embodiments, the method includes altering the selectivity for accepting the translocating nucleic acid molecule based on the number of nucleic acid molecules that have translocated into the second aqueous partition. In some embodiments, the method includes altering the selectivity for accepting the translocating nucleic acid molecule based on a ratio among nucleic acid molecules encoding two or more polypeptide members and that have been accepted into the second aqueous partition. In some embodiments, the rate at which nucleic acid molecules encoding polypeptide members of the specified mixture are accepted relative to each other is in proportion to the relative amount of the polypeptide members in the specified mixture. In some embodiments, the rate at which nucleic acid molecules encoding polypeptide members of the specified mixture are accepted into the second aqueous partition is measured based on the number of times a nucleic acid molecule encoding a polypeptide member of the specified mixture is accepted into the second aqueous partition relative to the number of times a nucleic acid molecule encoding another polypeptide member of the specified mixture is accepted into the second aqueous partition.

In some embodiments, the method includes dynamically controlling selectivity for accepting a nucleic acid molecule translocating through the nanopore from the first aqueous partition into the second aqueous partition. In some embodiments, the method includes dynamically controlling selectivity for accepting a nucleic acid molecule translocating through the nanopore from the first aqueous partition into the second aqueous partition across time, events, and/or across space (e.g., where a plurality of nanopores used). In some embodiments, dynamically controlling the selectivity includes continuously determining whether the selectivity needs to be altered. In some embodiments, dynamically controlling the selectivity includes determining at a defined time interval (e.g., over about 0.1-0.5 seconds, about 0.5-1 seconds, about 1-5 seconds, or about 5-10 seconds, or longer) whether to alter the selectivity. In some embodiments, dynamically controlling the selectivity includes determining after a defined number of events (e.g., after about 1-5, about 5-10, about 10-50, about 50-100, about 100-500, about 500-1000, or more events) whether to alter the selectivity.

In some embodiments, dynamically controlling the selectivity includes changing the selectivity during performance of the method (e.g., to achieve the specified mixture). In some embodiments, the selectivity for translocation of the nucleic acid molecule is altered based on the number of nucleic acid molecules that have translocated into the second aqueous partition. In some embodiments, dynamically controlling the selectivity includes changing the selectivity based on a difference in the desired amount or ratio of nucleic acid molecules encoding the polypeptide members of the specified mixture and the amount or ratio of accepted nucleic acid molecules encoding the polypeptide members of the specified mixture. In some embodiments, the method includes enumerating (e.g., storing in memory in accessible form) the nucleic acid molecules that have been accepted. In some embodiments, the method includes enumerating (e.g., store in memory in accessible form) the identity and/or the amount of the nucleic acid molecules that have been accepted. In some embodiments, the method includes controlling the selectivity for accepting the nucleic acid molecule translocating through the nanopore into the second aqueous partition based on at least the specified mixture (e.g., the composition of polypeptides in the specified mixture) and an enumeration of the nucleic acid molecules encoding the polypeptide members of the specified mixture that have been accepted. In some embodiments, the selectivity for translocation of the nucleic acid molecule is altered based on a ratio among nucleic acid molecules encoding two or more polypeptide members and that have translocated into the second aqueous partition.

In some embodiments, the method includes measuring an amount and/or level of activity of the at least one polypeptide member in the generated mixture of polypeptides. In some embodiments, the method include altering the selectivity for accepting the translocating nucleic acid molecule based on the measured amount and/or level of activity of the at least one polypeptide member in the generated mixture of polypeptides.

In some embodiments, the method includes altering the selectivity for accepting the translocating nucleic acid molecule into the second aqueous partition based on a ratio of activity among, or a collective activity level of, two or more polypeptide members in the generated mixture of polypeptides.

In some embodiments, the selectivity for accepting the translocating nucleic acid molecule into the second aqueous partition is altered due to a change in the composition of specified mixture of polypeptides. For example, a previously effective mixture of polypeptides generated by methods, systems, kits of some embodiments herein (e.g., a mixture of bacteriocins that was effective in keeping down growth of undesirable microbial organisms in an industrial culture, fermenter, pharmaceutical bioreactor, etc.) may become less effective, and a new mixture of polypeptides may be needed to maintain the same effectiveness, requiring a change in the selectivity for accepting the translocating nucleic acid molecule into the second aqueous partition to generate the new composition. In some embodiments, a composition of the specified mixture of polypeptides is altered. In some embodiments, a composition of the specified mixture of polypeptides is altered after generating the mixture of polypeptides encoded by the translocated nucleic acid molecules. In some embodiments, altering the specified mixture of polypeptides comprises removing or replacing one or more polypeptides members of the specified mixture, altering an amount of one or more polypeptides members in the specified mixture, and/or altering a proportion of one or more polypeptides members in the specified mixture.

In some embodiments, the first and second aqueous partitions are in communication (e.g., fluid communication) with each other via a plurality of the nanopores, wherein the method comprises determining a nucleotide sequence of the nucleic acid molecule translocating through each of two or more nanopores of the plurality of the nanopores and selectively accepting the nucleic acid molecule translocating through each of the two or more nanopores based on the determined nucleotide sequence of the corresponding translocating nucleic acid molecule.

Methods of the present disclosure, in some embodiments, can be performed using any of the systems and/or microfluidic devices described herein.

### Kits

Also provided is kit for making a composition comprising a specified mixture of polypeptides is provided. In some embodiments, the kit includes a microfluidic device as provided herein. In some embodiments, the kit includes a library of nucleic acids encoding a plurality of different polypeptides of a functional class, e.g., antimicrobial peptides, bacteriocins, cytokines, peptide toxins, signaling molecules, hormones. In some embodiments, the kit includes a nucleic acid expression solution, e.g., a translation and/or transcription solution. In some embodiments, the kit further comprises instructions for using the microfluidic device to make the composition comprising a specified mixture of polypeptides.

### EXAMPLES

### Example 1

A solution of RNA encoding the bacteriocins Subtilin and Bavaricin-MN, and the quorum sensing factor BsEDF is provided to a first partition that is separated from a second partition by a membrane. The membrane includes nanopores that are part of an adaptive nucleic acid conduit configured to selectively translocate RNA in the first partition into the second partition based on a determined sequence of a translocating RNA molecule. A RNA molecule translocating through the nanopore is sequenced while it is still translocating, using nanopore-based sequencing, and whether the translocating RNA molecule is allowed to translocate through the nanopore into the second partition is determined based at least on the determined sequence of the RNA molecule.

A specified mixture of polypeptides is determined to be Subtilin, Bavaricin-MN, and BsEDF in ratio of 1:2:1. The selectivity of translocation of RNA through the nanopore is controlled such that RNA encoding Subtilin, Bavaricin-MN, and BsEDF are accepted into the second partition at a ratio of 1:2:1 over a predetermined time period (e.g., the rate with which RNA encoding Subtilin, Bavaricin-MN, and BsEDF are accepted relative to each other is 1:2:1).

The RNA accepted into the second partition are collected and transferred to a translation solution. A mixture of polypeptides containing Subtilin, Bavaricin-MN, and BsEDF in a ratio of 1:2:1 is generated upon translation of the translocated RNA. The composition is added to an industrial feedstock to prevent the proliferation of undesired microbial organisms (via the bacteriocins), and to control the growth of genetically modified B. subtilis (via the BsEDF).

### Example 2

A solution of DNA encoding the bacteriocins Mundticin, Serracin-P, Thuricin-17, and Plantaricin J is provided to a first partition that is separated from a second partition by a membrane. The membrane includes nanopores that are part of an adaptive nucleic acid conduit configured to selectively translocate DNA in the first partition into the second partition based on a determined sequence of a translocating DNA molecule. A DNA molecule translocating through the nanopore is sequenced while it is still translocating, using nanopore-based sequencing, and whether the translocating DNA molecule is allowed to translocate through the nanopore into the second partition is determined based at least on the determined sequence of the DNA molecule.

It is determined that a ratio of the bacteriocins Mundticin, Serracin-P, Thuricin-17, and Plantaricin J of 1:2:3:4 is useful for targeting a population of undesired microbial cells in animal food during storage. The selectivity of translocation of DNA through the nanopore is controlled such that the DNA encoding Mundticin, Serracin-P, Thuricin-17, and Plantaricin J are accepted into the second partition at a ratio of 1:2:3:4 over a predetermined time period (e.g., the rate of translocation of RNA encoding Mundticin, Serracin-P, Thuricin-17, and Plantaricin J relative to each other is 1:2:3:4).

The DNA accepted into the second partition are collected and transferred to a transcription/translation solution. A mixture of polypeptides containing Mundticin, Serracin-P, Thuricin-17, and Plantaricin J in a ratio of 1:2:3:4 is generated upon transcription and translation of the translocated DNA. The composition comprising the bacteriocins in the 1:2:3:4 ratio is added to the animal food, thus targeting the population of undesired microbial cells in the animal food.

### Example 3

A microfluidic device that includes an input chamber and an output chamber separated by a membrane is provided. The input chamber contains a solution of nucleic acids, each encoding a bacteriocin selected from at least 5 of the bacteriocins listed in the even numbered sequences of SEQ ID NOS: 4-450 and the odd numbered sequences of SEQ ID NOS: 699-737. The membrane includes nanopores that allow single nucleic acid molecules to translocate from the input chamber to the output chamber when an appropriate voltage is applied across the membrane. The microfluidic device is configured to determine the sequence of a nucleic acid molecule translocating through the nanopore by nanopore-based sequencing. As each nucleotide of the sequence of nucleotides in the nucleic acid molecule passes through the nanopore, the current signature for the nucleotide (A, T, C, or G) is detected, and the sequence of nucleotides is determined in real-time by a processor. If the translocating nucleic acid molecule is determined to encode a bacteriocin of a specified mixture based on the determined sequence, and nucleic acid molecules encoding the bacteriocin is still needed in the output chamber based on the previously accepted amount of the nucleic acid molecules encoding the bacteriocin, the processor is instructed to cause the translocating nucleic acid molecule to be accepted and to allow it to complete translocation to the output chamber. If the translocating nucleic acid molecule is determined to encode a bacteriocin of the specified mixture based on the determined sequence, and a sufficient amount of the nucleic acid molecules encoding the bacteriocin has already translocated previously, the processor is instructed to cause the nucleic acid molecule to be bounced out by reversing the polarity of the voltage across the membrane. If the translocating nucleic acid molecule is determined to encode a bacteriocin that is not of the specified mixture based on the determined sequence, the processor is instructed to cause the nucleic acid molecule to be bounced out by reversing the polarity of the voltage across the membrane.

The nucleic acids that are accepted into the output chamber (e.g., the second aqueous partition) are contacted with a nucleic acid expression solution (e.g., a transcription and/or translation solution), and incubated at 37° C. thereby producing the encoded polypeptides from the translocated nucleic acid molecules in the amount and/or proportion of the specified mixture.

The device is connected electronically or wirelessly to a user input device such as a phone, touchscreen, keyboard, button, mouse, or computer. The processor selects bacteriocins based on user input entered into the user input device, or according to a pre-programmed set of instructions.

In at least some of the embodiments described herein, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

1. An in-vitro system for producing a specified mixture of polypeptides encoded by nucleic acids, comprising:
a membrane disposed between a first and second aqueous partitions, wherein the first aqueous partition comprises a plurality of nucleic acids encoding a plurality of different polypeptides, at least two of which correspond to polypeptide members of a specified mixture of two or more polypeptides, wherein the second aqueous partition comprises or is in fluid communication with a nucleic acid expression solution comprising at least a translation solution;
an adaptive nucleic acid conduit comprising a nanopore disposed in the membrane such that the first and second aqueous partitions are in communication with each other via the nanopore, wherein the adaptive nucleic acid conduit is configured to selectively accept a nucleic acid molecule of the plurality of nucleic acids translocating through the nanopore into the second aqueous partition based on a detected sequence of the translocating nucleic acid molecule; and
a selectivity controller configured to control the adaptive nucleic acid conduit's selectivity for accepting a nucleic acid molecule translocating through the nanopore from the first aqueous partition into the second aqueous partition, such that accepted nucleic acid molecules are allowed to be expressed in the nucleic acid expression solution to thereby generate of a mixture of two or more polypeptides encoded by the accepted nucleic acid molecules.

2. The system of claim 1, wherein the selectivity controller allows the adaptive nucleic acid conduit to accept the translocating nucleic acid molecule into the second aqueous partition if at least the identity of the polypeptide encoded by the translocating nucleic acid molecule is a polypeptide member of the specified mixture, and does not allow the adaptive nucleic acid conduit to accept nucleic acid molecules into the second aqueous partition if the identity of the polypeptide encoded by the translocating nucleic acid molecule is not a polypeptide member of the specified mixture.

3. The system of claim 1, wherein the selectivity controller allows the adaptive nucleic acid conduit to accept the translocating nucleic acid molecule into the second aqueous partition if at least the identity of the polypeptide encoded by the translocating nucleic acid molecule corresponds to a polypeptide member that is deficient in a composition of polypeptides generated by expressing the accepted nucleic acid molecules compared to the specified mixture of two or more polypeptides.

4. The system of claim 1, wherein the selectivity controller is configured to control the selectivity for accepting the nucleic acid molecule translocating through the nanopore into the second aqueous partition based on at least the specified mixture and an enumeration of the nucleic acid molecules encoding the polypeptide members of the specified mixture that have been accepted.

5. (canceled)

6. The system of claim 1, wherein the selectivity controller is configured to set the rate at which nucleic acid molecules encoding polypeptide members of the specified mixture are accepted into the second aqueous partition.

7. (canceled)

8. (canceled)

9. The system of claim 1, wherein the selectivity for accepting the nucleic acid molecule translocating through the nanopore into the second aqueous partition is altered based on the number of nucleic acid molecules that have been accepted into the second aqueous partition.

10. (canceled)

11. The system of claim 1, wherein the system is configured to measure an amount and/or level of activity of the at least one polypeptide member in the mixture of polypeptides encoded by the accepted nucleic acid molecules.

12. (canceled)

13. (canceled)

14. (canceled)

15. The system of claim 1, wherein the plurality of nucleic acids comprises one or more nucleic acids that encode at least one polypeptide that does not correspond to a polypeptide member of the specified mixture.

16. The system of claim 1, wherein the nucleic acid expression solution comprises a transcription solution and the translocated nucleic acid molecules are transcribed in the nucleic acid expression solution.

17. (canceled)

18. (canceled)

19. (canceled)

20. The system of claim 1, wherein each of the plurality of nucleic acids comprises a barcode that identifies the polypeptide encoded by the nucleic acid molecule, wherein the adaptive nucleic acid conduit is configured to selectively accept the nucleic acid molecule into the second aqueous partition based on the detected sequence of the barcode.

21. (canceled)

22. The system of claim 1, wherein the plurality of different polypeptides comprises a plurality of different antimicrobial peptides.

23. The system of claim 22, wherein the antimicrobial peptides comprise bacteriocins.

24. (canceled)

25. The system of claim 1, wherein the second aqueous partition is in fluid communication with a tissue, a wound, a host microbiome, industrial culture, feedstock, fermenter, or a food, pharmaceutical, or cosmetic manufacturing environment.

26. The system of claim 1, comprising a microfluidic device comprising the first and second aqueous partitions; the membrane disposed between the first and second aqueous partitions; and the nanopore disposed in the membrane.

27. The system of claim 1, wherein the nanopore comprises MspA, alpha-hemolysin, anthrax toxin, leukocidins, OmpF, OmpG, OmpATb, NalP, and/or lysenin.

28. The system of claim 1, wherein the adaptive nucleic acid conduit comprises a helicase or a polymerase associated with the nanopore.

29. The system of claim 1, wherein the membrane comprises a lipid bilayer.

30. The system of claim 1, wherein the first and second aqueous partitions are comprised in a chamber comprising the membrane disposed therein so as to separate the chamber into two portions, one of which comprises the first aqueous partition and the other comprises the second aqueous partition.

31. (canceled)

32. The system of claim 1, wherein the nucleic acid molecule can translocate from the first aqueous partition to the second aqueous partition only via the nanopore comprised in the adaptive nucleic acid conduit.

33. A method of selecting nucleic acids for producing a specified mixture of polypeptides encoded by the nucleic acids, the method comprising:
(a) providing in a first aqueous partition a plurality of nucleic acids encoding a plurality of different polypeptides, wherein the plurality of nucleic acids comprises nucleic acid molecules that encode at least two polypeptide members of a specified mixture of two or more polypeptides, wherein a membrane is disposed between the first aqueous partition and a second aqueous partition, wherein the first and second aqueous partitions are in communication with each other via a nanopore configured such that nucleic acid molecules of the plurality of nucleic acids can translocate from the first aqueous partition through the nanopore into the second aqueous partition;
(b) sequencing a nucleic acid molecule of the plurality of nucleic acids translocating through the nanopore using nanopore-based sequencing to determine a nucleotide sequence of the translocating nucleic acid molecule; and
(c) selectively accepting a nucleic acid molecule of the plurality of nucleic acids translocating through the nanopore into the second aqueous partition based on the determined nucleotide sequence, wherein the selectivity for accepting the translocating nucleic acid molecule into the second aqueous partition through the nanopore is controllable such that the accepted nucleic acid molecules, when expressed, produce a composition comprising the specified mixture of two or more polypeptides.

34-68. (canceled)


SEQUENCE LISTING (750 sequences)

SEQ ID NO: 1 - protein, 40 aa, Enterococcus faecalis
WLPPAGLLGRCGRWFRPWLLWLQSGAQYKWLGNLFGLGPK

SEQ ID NO: 2 - protein, 5 aa, synthetic construct
  REGION 1..5: note = N-terminal motif of class terminal IIa
  VARIANT 3: note = Xaa = any amino acid
YGXGV

SEQ ID NO: 3 - protein, 135 aa, synthetic construct
  REGION 1..135: note = Hybrid bacteriocin Ent35-MccV
GKYYGNGVSCNKKGCSVDWGRAIGIIGNNSAANLATGGAAGWKSGGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 4 - protein, 46 aa, Lactobacillus acidophilus
MISSHQKTLTDKELALISGGKTHYPTNAWKSLWKGFWESLRYTDGF

SEQ ID NO: 5 - DNA, 141 bp, Lactobacillus acidophilus
  source 1..141: mol_type = genomic DNA
atgatttcatctcatcaaaaaacgttaactgataaagaattagcattaatttctggggggaaaacgcactacccgactaatgcatggaaaagtctttggaaaggtttctgggaaagccttcgttatactgacggtttttag

SEQ ID NO: 6 - protein, 81 aa, Lactobacillus acidophilus
MISMISSHQKTLTDKELALISGGKTYYGTNGVHCTKKSLWGKVRLKNVIPGTLCRKQSLPIKQDLKILLGWATGAFGKTFH

SEQ ID NO: 7 - DNA, 246 bp, Lactobacillus acidophilus
  source 1..246: mol_type = genomic DNA
atgatttcaatgatttcatctcatcaaaaaacgttaactgataaagaattagcattaatttctggggggaaaacgtactatggtactaatggtgtgcattgtactaaaaagagtctttggggtaaagtacgcttaaaaaacgtgattcctggaactctttgtcgtaagcaatcgttgccgatcaaacaggatttaaaaattttactgggctgggctacaggtgcttttggcaagacatttcattaa

SEQ ID NO: 8 - protein, 60 aa, Lactobacillus acidophilus
MDKKTKILFEVLYIICIIGPQFILFVTAKNNMYQLVGSFVGIVWFSYIFWYIFFKQHKKM

SEQ ID NO: 9 - DNA, 183 bp, Lactobacillus acidophilus
  source 1..183: mol_type = genomic DNA
atggataagaaaacaaaaatattatttgaagtattatacatcatctgtataataggccctcaatttatattatttgtgactgcaaaaaacaatatgtatcagttggtgggttcgtttgttggaatagtatggttttcgtatattttttggtatatttttttcaaacaacataaaaaaatgtag

SEQ ID NO: 10 - protein, 65 aa, Lactobacillus gasseri
MALKTLEKHELRNVMGGNKWGNAVIGAATGATRGVSWCRGFGPWGMTACALGGAAIGGYLGYKSN

SEQ ID NO: 11 - DNA, 198 bp, Lactobacillus gasseri
  source 1..198: mol_type = genomic DNA
atggctttaaaaacattagaaaaacatgaattaagaaatgtaatgggtggaaacaagtgggggaatgctgtaataggagctgctacgggagctactcgcggagtaagttggtgcagaggattcggaccatggggaatgactgcctgtgcgttaggaggtgctgcaattggaggatatctgggatataagagtaattaa

SEQ ID NO: 12 - protein, 51 aa, Staphylococcus aureus [sequence omitted]

SEQ ID NO: 13 - DNA, 156 bp, Staphylococcus aureus
  source 1..156: mol_type = genomic DNA
atgagttggttaaattttttaaaatacatcgctaaatatggcaaaaaagcggtatctgctgcttggaagtacaaaggtaaagtattagaatggcttaatgttggtcctactcttgaatgggtatggcaaaaattaaagaaaattgctggattataa

SEQ ID NO: 14 - protein, 61 aa, Enterococcus avium
MTRSKKLNLREMKNVVGGTYYGNGVSCNKKGCSVDWGKAISIIGNNSAANLATGGAAGWKS

SEQ ID NO: 15 - DNA, 186 bp, Enterococcus avium
  source 1..186: mol_type = genomic DNA
atgacaagatcaaaaaaattaaatttacgcgaaatgaagaatgttgttggtggtacctactatggaaatggtgtatcttgtaacaagaaaggctgttcagttgactggggcaaagccatcagtattataggaaataattccgcagcaaacttagcaactggtggtgctgctggttggaagtcataa

SEQ ID NO: 16 - protein, 67 aa, Enterococcus faecalis
MKKKLVICGIIGIGFTALGTNVEAATYYGNGLYCNKQKCWVDWNKASREIGKIIVNGWVQHGPWAPR

SEQ ID NO: 17 - DNA, 204 bp, Enterococcus faecalis
  source 1..204: mol_type = genomic DNA
atgaaaaagaaattagttatttgtggcattattgggattggttttacagcattaggaacaaatgtagaagctgctacgtattacggaaatggtttatattgtaataagcaaaaatgttgggtagactggaataaagcttcaagggaaattggaaaaattattgttaatggttgggtacaacatggcccttgggctcctagatag

SEQ ID NO: 18 - protein, 51 aa, Lactococcus lactis
MKEQNSFNLLQEVTESELDLILGAKGGSGVIHTISHEVIYNSWNFVFTCCS

SEQ ID NO: 19 - DNA, 156 bp, Lactococcus lactis
  source 1..156: mol_type = genomic DNA
atgaaagaacaaaactcttttaatcttcttcaagaagtgacagaaagtgaattggaccttattttaggtgcaaaaggcggcagtggagttattcatacaatttctcatgaagtaatatataatagctggaactttgtatttacttgctgctcttaa

SEQ ID NO: 20 - protein, 74 aa, Enterococcus faecium
MKKKVLKHCVILGILGTCLAGIGTGIKVDAATYYGNGLYCNKEKCWVDWNQAKGEIGKIIVNGWVNHGPWAPRR

SEQ ID NO: 21 - DNA, 225 bp, Enterococcus faecium
  source 1..225: mol_type = genomic DNA
atgaaaaagaaagtattaaaacattgtgttattctaggaatattaggaacttgtctagctggcatcggtacaggaataaaagttgatgcagctacttactatggaaatggtctttattgtaacaaagaaaaatgttgggtagattggaatcaagctaaaggagaaattggaaaaattattgttaatggttgggttaatcatggtccatgggcacctagaaggtag

SEQ ID NO: 22 - protein, 50 aa, Clostridium botulinum
MQKPEIISADLGLCAVNEFVALAAIPGGAATFAVCQMPNLDEIVSNAAYV

SEQ ID NO: 23 - DNA, 153 bp, Clostridium botulinum
  source 1..153: mol_type = genomic DNA
atgcaaaaaccagaaattattagtgctgatttagggctttgtgcagttaatgaatttgtagctcttgctgccattcctggtggtgctgctacatttgcagtatgccaaatgccaaacttggatgagattgttagtaatgcagcatatgtttaa

SEQ ID NO: 24 - protein, 58 aa, Streptococcus equinus
MMNATENQIFVETVSDQELEMLIGGADRGWIKTLTKDCPNVISSICAGTIITACKNCA

SEQ ID NO: 25 - DNA, 177 bp, Streptococcus equinus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcttgtaaaaattgtgcttaa

SEQ ID NO: 26 - protein, 77 aa, Brochothrix campestris
MHKVKKLNNQELQQIVGGYSSKDCLKDIGKGIGAGTVAGAAGGGLAAGLGAIPGAFVGAHFGVIGGSAACIGGLLGN

SEQ ID NO: 27 - DNA, 234 bp, Brochothrix campestris
  source 1..234: mol_type = genomic DNA
atgcacaaggtaaaaaaattaaacaatcaagagttacaacagatcgtgggaggttacagttcaaaagattgtctaaaagatattggtaaaggaattggtgctggtacagtagctggggcagccggcggtggcctagctgcaggattaggtgctatcccaggagcattcgttggagcacattttggagtaatcggcggatctgccgcatgcattggtggattattaggtaactag

SEQ ID NO: 28 - protein, 80 aa, Butyrivibrio fibrisolvens
MSKKQIMSNCISIALLIALIPNIYFIADKMGIQLAPAWYQDIVNWVSAGGTLTTGFAIIVGVTVPAWIAEAAAAFGIASA

SEQ ID NO: 29 - DNA, 243 bp, Butyrivibrio fibrisolvens
  source 1..243: mol_type = genomic DNA
atgagtaaaaaacaaattatgagtaactgtatatcaattgcattattaatagcactaattcctaatatctattttattgcagataaaatgggaattcagttagcacctgcttggtatcaagatattgtgaattgggtatctgctggtggaacacttactactggttttgcgattattgtaggagttacagtaccggcatggatagcagaagcagctgcagcttttggtatagcttcagcatga

SEQ ID NO: 30 - protein, 48 aa, Butyrivibrio fibrisolvens
MNKELNALTNPIDEKELEQILGGGNGVIKTISHECHMNTWQFIFTCCS

SEQ ID NO: 31 - DNA, 147 bp, Butyrivibrio fibrisolvens
  source 1..147: mol_type = genomic DNA
atgaacaaagaacttaatgcacttacaaatcctattgacgagaaggagcttgagcagatcctcggtggtggcaatggtgtcatcaagacaatcagccacgagtgccacatgaacacatggcagttcattttcacatgttgctcttaa

SEQ ID NO: 32 - protein, 66 aa, Carnobacterium maltaromaticum
MNSVKELNVKEMKQLHGGVNYGNGVSCSKTKCSVNWGQAFQERYTAGINSFVSGVASGAGSIGRRP

SEQ ID NO: 33 - DNA, 201 bp, Carnobacterium maltaromaticum
  source 1..201: mol_type = genomic DNA
atgaatagcgtaaaagaattaaacgtgaaagaaatgaaacaattacacggtggagtaaattatggtaatggtgtttcttgcagtaaaacaaaatgttcagttaactggggacaagcctttcaagaaagatacacagctggaattaactcatttgtaagtggagtcgcttctggggcaggatccattggtaggagaccgtaa

SEQ ID NO: 34 - protein, 61 aa, Carnobacterium maltaromaticum
MKSVKELNKKEMQQINGGAISYGNGVYCNKEKCWVNKAENKQAITGIVIGGWASSLAGMGH

SEQ ID NO: 35 - DNA, 186 bp, Carnobacterium maltaromaticum
  source 1..186: mol_type = genomic DNA
atgaaaagcgttaaagaactaaataaaaaagaaatgcaacaaattaatggtggagctatctcttatggcaatggtgtttattgtaacaaagagaaatgttgggtaaacaaggcagaaaacaaacaagctattactggaatagttatcggtggatgggcttctagtttagcaggaatgggacattaa

SEQ ID NO: 36 - protein, 71 aa, Carnobacterium maltaromaticum
MNNVKELSIKEMQQVTGGDQMSDGVNYGKGSSLSKGGAKCGLGIVGGLATIPSGPLGWLAGAAGVINSCMK

SEQ ID NO: 37 - DNA, 216 bp, Carnobacterium maltaromaticum
  source 1..216: mol_type = genomic DNA
atgaataatgtaaaagagttaagtattaaagaaatgcaacaagttactggtggagaccaaatgtcagatggtgtaaattatggaaaaggctctagcttatcaaaaggtggtgccaaatgtggtttagggatcgtcggcggattagctactatcccttcaggtcctttaggctggttagccggagcagcaggtgtaattaatagctgtatgaaataa

SEQ ID NO: 38 - protein, 64 aa, Carnobacterium maltaromaticum
MLYELVAYGIAQGTAEKVVSLINAGLTVGSIISILGGVTVGLSGVFTAVKAAIAKQGIKKAIQL

SEQ ID NO: 39 - DNA, 195 bp, Carnobacterium maltaromaticum
  source 1..195: mol_type = genomic DNA
atgttatatgaattagttgcatatggtatcgcacaaggtacagctgaaaaggttgtaagtctaattaacgcaggtttaacagtagggtctattatttcaattttgggtggggtcacagtcggtttatcaggtgtcttcacagcagttaaagcagcaattgctaaacaaggaataaaaaaagcaattcaattataa

SEQ ID NO: 40 - protein, 836 aa, Pectobacterium carotovorum
  source 1..836: note = subsp. carotovorum
MIKYRLYAPNDGDTMTVSGGGGWVSNDDRKGGNDRDNGKGGSAVDFSKNPEKQAIVNPYLAIAIPMPVYPLYGKLGFTINTTAIETELANVRAAINTKLATLSAVIGRSLPVVGRVFGVTAAGMWPSSTAPSSLDSIYNQAHQQALAQLAAQQGVLNKGYNVTAMPAGFVSSLPVSEIKSLPTAPASLLAQSVINTELSQRQLALTQPTTNAPVANIPVVKAEKTAMPGVYSAKIIAGEPAFQIKVDNTKPALAQNPPKVKDDIQVSSFLSSPVADTHHAFIDFGSDHEPVYVSLSKIVTAEEEKKQVEEAKRREQEWLLRHPITAAERKLTEIRQVISFAQQLKESSVATISEKTKTVAVYQEQVNTAAKNRDNFYNQNRGLLSAGITGGPGYPIYLALWQTMNNFHQAYFRANNALEQESHVLNLARSDLAKAEQLLAENNRLQVETERTLAEEKEIKRNRVNVSTFGTVQTQLSKLLSDFYAVTSLSQSVPSGALASFSYNPQGMIGSGKIVGKDVDVLFSIPVKDIPGYKSPINLDDLAKKNGSLDLPIRLAFSDENGERVLRAFKADSLRIPSSVRGVAGSYDKNTGIFSAEIDGVSSRLVLENPAFPPTGNVGNTGNTAPDYKALLNTGVDVKPVDKITVTVTPVADPVDIDDYIIWLPTASGSGVEPIYVVFNSNPYGGTEKGKYSKRYYNPDKAGGPILELDWKNVKIDHAGVDNVKLHTGRFKASVENKVMIERLENILNGQITATDTDKRFYTHELRELNRYRNLGIKDGEVPSSIQEESAVWNDTHTATLEDYKINEKEQPLYTDAALQAAYEQELKDALGGKHG

SEQ ID NO: 41 - DNA, 2511 bp, Pectobacterium carotovorum
  source 1..2511: mol_type = genomic DNA; note = subsp. carotovorum
atgattaaataccgtttatatgctccaaatgatggagacaccatgacagtgagtggtggtggtggttgggtttcaaacgatgatcgcaaaggtggtaatgacagggacaatggcaaaggtggttctgccgttgattttagtaaaaatccagaaaagcaggctatcgttaatccctatttggcaatcgcgataccgatgccggtctaccctctttatggaaagctagggttcacaataaatacgacggcaattgagactgaactcgcaaatgtcagagcagcaattaacactaaacttgcaacactcagtgcagtgattggcagatcacttccggtcgttgggcgggtatttggtgttactgccgccggaatgtggccttctagtaccgctcccagtagtctcgattctatatacaatcaagcacatcagcaggctttagcccagttagctgctcaacagggagtattaaataaagggtataacgttacagcaatgcctgcaggtttcgtcagcagtttgcctgttagtgaaatcaaatcattgccaacagctcccgccagtttactggcacaaagtgtgattaataccgaactttcccagcgtcaactggctcttactcagcccacgacgaatgcaccagtcgcgaatattcccgtagttaaagcagagaaaacagcaatgccaggtgtgtattcagcgaaaattattgctggtgagcctgcattccaaatcaaggtcgataataccaaacctgctttggcacagaatccgccgaaagtaaaagatgatattcaggtatcttctttcctttcctcgccagtagctgatacgcaccatgcatttattgattttggcagcgatcatgaaccggtatacgtgtctctttcaaagatcgtgacagccgaggaggagaaaaaacaggttgaagaggccaagcgccgtgagcaggagtggttgttgcgtcatccaattacagctgcggagcgaaaattaactgaaatccgccaagtgatctcttttgctcaacagctaaaagaaagctctgtcgcaaccatttcagaaaaaactaaaactgttgcggtttaccaagaacaggtgaataccgctgcaaaaaatcgcgacaatttttataatcaaaatagaggtctgttaagtgcgggtataactgggggaccgggatatcctatttatcttgctttatggcaaacgatgaataactttcatcaggcttatttcagagcaaataatgcattggaacaagagagtcatgttctgaacctggctcgttctgatctggctaaggctgagcaattgcttgctgagaataatcgacttcaggttgaaacggagcgaacgcttgccgaagaaaaagagataaaacgcaacagggttaatgtatcaacatttggcacagtgcaaactcaacttagtaaattgctgtcagatttttatgctgttacatcactttcccaaagtgttccttcgggggcattagcctctttttcatataatccacaagggatgattggcagcggtaagattgttgggaaggatgtcgatgttttattttccatcccagtaaaagatattccgggatataaatctcctattaacttggacgatttagccaagaaaaatggaagtctggatcttcccattcgtctggcattttctgatgagaatggagaaagggttcttcgggcattcaaagcggatagtctgcgaatcccttcgagtgtcagaggtgtagcgggcagttatgacaaaaatacgggtatttttagtgcagaaattgatggtgtttcatctcgccttgtactggaaaacccagcgtttcctccgaccggaaatgtcggtaatacgggtaatactgcacctgactataaagcattactgaatactggtgttgatgttaaacctgttgataaaatcacagttacggtaacaccagttgctgatccagtggatattgatgactatataatctggttgccaactgcgtctggttctggcgtggaacccatttatgtcgtgtttaacagtaatccgtatggtgggacggaaaaaggaaaatatagcaaacgttattataatccagataaggcaggcggtccgatcttggagctggattggaaaaacgttaagattgaccatgcaggtgtggacaatgttaaattacacacagggcgtttcaaagcgtcggttgaaaacaaagtgatgattgaacgtttggaaaacatactgaatggtcaaatcacggccacggatactgacaagcgattctatacgcatgaattaagagagttaaaccgctacagaaatttaggcatcaaagacggtgaagtgcctagtagcattcaagaagaaagcgctgtttggaacgacacacacacagcgacgcttgaagactacaaaattaatgagaaagagcaaccgttgtacactgatgctgctttgcaggcagcctacgaacaggaactcaaagacgcattaggagggaaacatggctaa

SEQ ID NO: 42 - protein, 74 aa, Bacillus cereus
MENLQMLTEEELMEIEGGGWWNSWGKCVAGTIGGAGTGGLGGAAAGSAVPVIGTGIGGAIGGVSGGLTGAATFC

SEQ ID NO: 43 - DNA, 225 bp, Bacillus cereus
  source 1..225: mol_type = genomic DNA
atggaaaacttacaaatgttaactgaagaagaattaatggaaattgaaggtggaggctggtggaatagctggggtaaatgtgttgctggaactatcggtggagctggaactggtggtttaggtggagctgctgcaggttcagctgttccggttattggtactggtattggtggcgctattggtggagttagcggtggccttacaggtgcagctactttttgctaa

SEQ ID NO: 44 - protein, 78 aa, Streptoverticillium griseoverticillatum
MTASILQQSVVDADFRAALLENPAAFGASAAALPTPVEAQDQASLDFWTKDIAATEAFACRQSCSFGPFTFVCDGNTK

SEQ ID NO: 45 - DNA, 237 bp, Streptoverticillium griseoverticillatum
  source 1..237: mol_type = genomic DNA
atgaccgcttccattcttcagcagtccgtcgtggacgccgacttccgcgcggcgctgcttgagaaccccgccgccttcggcgcttccgccgcggccctgcccacgcccgtcgaggcccaggaccaggcgtcccttgacttctggaccaaggacatcgccgccacggaagccttcgcctgccgccagagctgcagcttcggcccgttcaccttcgtgtgcgacggcaacaccaagtaa

SEQ ID NO: 46 - protein, 76 aa, Geobacillus kaustophilus
MSLLALVAGTLGVSQSIATTVVSIVLTGSTLISIILGITAILSGGVDAILEIGWSAFVATVKKIVAERGKAAAIAW

SEQ ID NO: 47 - DNA, 231 bp, Geobacillus kaustophilus
  source 1..231: mol_type = genomic DNA
atgagtttgctggcgcttgttgccgggacgctcggcgtgtcacagtcaatcgcgacgacggttgtttcgattgtgttgaccggctccactctcatttctattattcttgggatcaccgctattttgtcaggtggagtcgacgccattttggaaattgggtggtcagcttttgtcgcgacggtgaaaaaaatagtggcggaacgaggaaaagcggcagcgattgcatggtaa

SEQ ID NO: 48 - protein, 309 aa, Clostridium tyrobutyricum
MRKVFLRSIISTLVMCAFVSSSFSVNADESKPNDEKIINNIENVTTTKDIVKSNKNNIVYLDEGVMSIPLSGRKPIAIKDDNNKEDLTVTLPIKNTGDISKISSNGTILYKNNSSNSSNIALQPKNDGFKALININDKLANKEYEFTFNLPKNSKLISAATYLGKEYDTKEVFVVDKNNIITSIISPAWAKDANGHNVSTYYKIVSNNKLVQVVEFTENTAFPVVADPNWTKIGKCAGSIAWAIGSGLFGGAKLIKIKKYIAELGGLQKAAKLLVGATTWEEKLHAGGYALINLAAELTGVAGIQANCF

SEQ ID NO: 49 - DNA, 930 bp, Clostridium tyrobutyricum
  source 1..930: mol_type = genomic DNA
ttgagaaaagtatttttaagatcaataatttcaacattagttatgtgtgcatttgtttcaagcagcttttcagtaaatgcggatgaaagcaaaccaaatgatgaaaaaataattaataacatagaaaacgttactactactaaagatattgtaaaaagtaataaaaataatattgtatatttagatgaaggtgtaatgagtattccattgtctgggagaaaacccattgctattaaagatgataataataaagaagatttaactgttacattacctattaagaatactggagatatatctaaaattagtagtaatggtactattctgtataaaaataatagtagtaattcatctaatatagctttacaacctaaaaatgatggatttaaggctttaataaatattaatgataagttagctaataaagaatatgaatttacatttaatttacccaaaaacagtaaattaattagtgctgccacatatttgggtaaagaatatgatacaaaagaagtatttgtagtagacaaaaataatataattacgagtattattagtccagcttgggctaaagatgcaaatggacataatgtttctacttattataagatagtatcgaataataaattagtacaagttgttgaattcacagaaaatactgcattcccggtggtagctgatcctaattggactaaaattgggaaatgcgctgggtcaatagcatgggctataggttctggcctttttggtggagcaaagctaattaaaataaaaaaatatatagcagagcttggaggacttcaaaaagcagctaaattattagttggtgcaaccacttgggaagaaaaattacacgcaggcggttatgcattaattaacttagctgctgagctaacaggtgtagcaggtatacaagcaaattgtttttaa

SEQ ID NO: 50 - protein, 62 aa, Bacillus coagulans
MKKIEKLTEKEMANIIGGKYYGNGVTCGKHSCSVDWGKATTCIINNGAMAWATGGHQGTHKC

SEQ ID NO: 51 - DNA, 189 bp, Bacillus coagulans
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacctgcataatcaataatggagctatggcatgggctactggtggacatcaaggtactcataaatgctag

SEQ ID NO: 52 - protein, 490 aa, Escherichia coli
MDKVTDNSPDVESTESTEGSFPTVGVDTGDTITATLATGTENVGGGGGAFGGASESSAAIHATAKWSTAQLKKHQAEQAARAAAAEAALAKAKSQRDALTQRLKDIVNDALRANAARSPSVTDLAHANNMAMQAEAERLRLAKAEQKAREEAEAAEKALREAERQRDEIARQQAETAHLLAMAEAAEAEKNRQDSLDEEHRAVEVAEKKLAEAKAELAKAESDVQSKQAIVSRVAGELENAQKSVDVKVTGFPGWRDVQKKLERQLQDKKNEYSSVTNALNSAVSIRDAKKTEVQNAEIKLKEAKDALEKSQVKDSVDTMVGFYQYITEQYGEKYSRIAQDLAEKAKGSKFNSVDEALAAFEKYKNVLDKKFSKVDRDDIFNALESITYDEWAKHLEKISRALKVTGYLSFGYDVWDGTLKGLKTGDWKPLFVTLEKSAVDFGVAKIVALMFSFIVGAPLGFWGIAIITGIVSSYIGDDELNKLNELLGI

SEQ ID NO: 53 - DNA, 1473 bp, Escherichia coli
  source 1..1473: mol_type = genomic DNA
atggataaagtcactgataattctccagatgtggagagcacagaatctactgaggggtcattcccaactgttggggttgatactggcgatacgattacagcgacgcttgcaactggaactgaaaatgttggtggaggcggtggagcatttggtggggccagtgaaagttctgctgcgatacatgcaaccgctaaatggtctaccgcgcagttgaaaaaacatcaggctgaacaggctgcccgtgctgctgcggctgaggcagcattggcaaaagcgaaatctcagcgtgatgccctgactcaacgtctcaaggatattgttaatgacgctttacgtgctaatgccgctcgtagtccatcagtaactgaccttgctcatgccaataatatggcaatgcaggcagaggctgagcgtttgcgccttgcgaaggcagagcaaaaagcccgtgaagaagctgaagcagcagaaaaagcgctccgggaagcagaacgccaacgtgatgagattgcccgccaacaggctgaaaccgcgcatttgttagcaatggcggaggcagcagaggctgagaaaaatcgacaggattctcttgatgaagagcatcgggctgtggaagtggcagagaagaagctggctgaggctaaagctgaactggcgaaggccgaaagcgatgtacagagtaagcaagcgattgtttccagagttgcaggggagcttgaaaacgctcaaaaaagtgttgatgtgaaggttaccggatttcctggatggcgtgatgttcagaaaaaactggagagacaattgcaggataagaagaatgaatattcgtcagtgacgaatgctcttaattctgctgttagcattagagatgctaaaaaaacagaagttcagaatgctgagataaaattaaaagaagctaaggatgctcttgagaagagtcaggtaaaagactctgttgatactatggttgggttttatcaatatataaccgaacaatatggggaaaaatattccagaatagctcaggatttagctgaaaaggcgaagggtagtaaatttaatagtgttgatgaagcacttgctgcatttgaaaagtataaaaatgtactggataagaaattcagtaaggttgatagggatgatatttttaatgctttagagtctattacttatgatgagtgggccaagcatctagaaaagatctctagggctcttaaggttactggatatttgtctttcgggtatgatgtatgggatggtaccctaaagggattaaaaacaggagactggaagcctttatttgtcactctggagaagagcgcggtagatttcggcgtggcaaaaattgtggcattaatgtttagttttattgttggtgcgcctcttggcttctggggaattgcaattatcacaggtattgtttcttcttacataggggatgatgagttgaacaagcttaatgaattactaggtatttaa

SEQ ID NO: 54 - protein, 522 aa, Escherichia coli
METAVAYYKDGVPYDDKGQVIITLLNGTPDGSGSGGGGGKGGSKSESSAAIHATAKWSTAQLKKTQAEQAARAKAAAEAQAKAKANRDALTQRLKDIVNEALRHNASRTPSATELAHANNAAMQAEDERLRLAKAEEKARKEAEAAEKAFQEAEQRRKEIEREKAETERQLKLAEAEEKRLAALSEEAKAVEIAQKKLSAAQSEVVKMDGEIKTLNSRLSSSIHARDAEMKTLAGKRNELAQASAKYKELDELVKKLSPRANDPLQNRPFFEATRRRVGAGKIREEKQKQVTASETRINRINADITQIQKAISQVSNNRNAGIARVHEAEENLKKAQNNLLNSQIKDAVDATVSFYQTLTEKYGEKYSKMAQELADKSKGKKIGNVNEALAAFEKYKDVLNKKFSKADRDAIFNALASVKYDDWAKHLDQFAKYLKITGHVSFGYDVVSDILKIKDTGDWKPLFLTLEKKAADAGVSYVVALLFSLLAGTTLGIWGIAIVTGILCSYIDKNKLNTINEVLGI

SEQ ID NO: 55 - DNA, 1569 bp, Escherichia coli
  source 1..1569: mol_type = genomic DNA
atggaaaccgcggtagcgtactataaagatggtgttccttatgatgataagggacaggtaattattactcttttgaatggtactcctgacgggagtggctctggcggcggaggtggaaaaggaggcagtaaaagtgaaagttctgcagctattcatgcaactgctaaatggtctactgctcaattaaagaaaacacaggcagagcaggctgcccgggcaaaagctgcagcggaagcacaggcgaaagcaaaggcaaacagggatgcgctgactcagcgcctgaaggatatcgtgaatgaggctcttcgtcacaatgcctcacgtacgccttcagcaacagagcttgctcatgctaataatgcagctatgcaggcggaagacgagcgtttgcgccttgcgaaagcagaagaaaaagcccgtaaagaagcggaagcagcagaaaaggcttttcaggaagcagaacaacgacgtaaagagattgaacgggagaaggctgaaacagaacgccagttgaaactggctgaagctgaagagaaacgactggctgcattgagtgaagaagctaaagctgttgagatcgcccaaaaaaaactttctgctgcacaatctgaagtggtgaaaatggatggagagattaagactctcaattctcgtttaagctccagtatccatgcccgtgatgcagaaatgaaaacgctcgctggaaaacgaaatgaactggctcaggcatccgctaaatataaagaactggatgagctggtcaaaaaactatcaccaagagccaatgatccgcttcagaaccgtcctttttttgaagcaaccagacgacgggttggggccggtaagattagagaagaaaaacaaaaacaggtaacagcatcagaaacacgtattaaccggataaatgctgatataactcagatccagaaggctatttctcaggtcagtaataatcgtaatgccggtatcgctcgtgttcatgaagctgaagaaaatttgaaaaaagcacagaataatctccttaattcacagattaaggatgctgttgatgcaacagttagcttttatcaaacgctgactgaaaaatatggtgaaaaatattcgaaaatggcacaggaacttgctgataagtctaaaggtaagaaaatcggcaatgtgaatgaagctctcgctgcttttgaaaaatacaaggatgttttaaataagaaattcagcaaagccgatcgtgatgctatttttaatgcgttggcatcggtgaagtatgatgactgggctaaacatttagatcagtttgccaagtacttgaagattacggggcatgtttcttttggatatgatgtggtatctgatatcctaaaaattaaggatacaggtgactggaagccactatttcttacattagagaagaaagctgcagatgcaggggtgagttatgttgttgctttactttttagcttgcttgctggaactacattaggtatttggggtattgctattgttacaggaattctatgctcctatattgataagaataaacttaatactataaatgaggtgttagggatttaa

SEQ ID NO: 56 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGI

SEQ ID NO: 57 - DNA, 1881 bp, Escherichia coli
  source 1..1881: mol_type = genomic DNA
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatgattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatccgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttaa

SEQ ID NO: 58 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLKTARNALTRAEQQLTQQKNTPDGKTIVSPEKFPGRSSTNHSIVVSGDPRFAGTIKITTSAVIDNRANLNYLLTHSGLDYKRNILNDRNPVVTEDVEGDKKIYNAEVAEWDKLRQRLLDARNKITSAESAINSARNNVSARTNEQKHANDALNALLKEKENIRSQLADINQKIAEEKRKRDEINMVKDAIKLTSDFYRTIYDEFGKQASELAKELASVSQGKQIKSVDDALNAFDKFRNNLNKKYNIQDRMAISKALEAINQVHMAENFKLFSKAFGFTGKVIERYDVAVELQKAVKTDNWRPFFVKLESLAAGRAASAVTAWAFSVMLGTPVGILGFAIIMAAVSALVNDKFIEQVNKLIGI

SEQ ID NO: 59 - DNA, 1881 bp, Escherichia coli
  source 1..1881: mol_type = genomic DNA
atgtctgaccctgtacgtattacaaatcccggtgcagaatcgctgggatatgattcagatggccatgaaattatggccgttgatatttatgtaaaccctccacgtgtcgatgtctttcatggtaccccgcctgcatggagttccttcgggaacaaaaccatctggggtggaaacgagtgggtcgatgattccccaacccgaagtgatatcgaaaaaagggacaaggaaatcacagcgtacaaaaacacgctcagcgcgcagcagaaagagaatgagaataagcgtactgaagctggaaaacgcctttctgcggcaattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgatgccctccggctgcatacagagagccggatgctgtttgctgatgctgattctcttcgtatatctccccgcgaggccaggtcgttaatcgaacaggctgaaaaacggcagaaggatgcgcagaacgcagacaagaaggccgctgatatgcttgctgaatacgagcgcagaaaaggtattctggacacgcggttgtcagagctggaaaaaaatggcggggcagcccttgccgttcttgatgcacaacaggcccgtctgctcgggcagcagacacggaatgacagggccatttcagaggcccggaataaactcagttcggtgacggaatcgcttaagacggcccgtaatgcattaaccagagctgaacaacagctgacgcaacagaaaaacacgcctgacggcaaaacgatagtttcccctgaaaaattcccggggcgttcatcaacaaatcattctattgttgtgagtggtgatccgaggtttgccggtacgataaaaatcacaaccagcgcggtcatcgataaccgtgcaaacctgaattatcttctgacccattccggtctggactataaacgcaatattctgaatgaccggaatccggtggtgacagaggatgtggaaggtgacaagaaaatttataatgctgaagttgctgaatgggataagttacggcaacgattgcttgatgccagaaataaaatcacctctgctgaatctgcgataaattcggcgagaaataacgtcagtgccagaacaaatgaacaaaagcatgcaaatgacgctcttaatgccctgttgaaggaaaaagagaatatccgtagccagcttgctgacatcaatcagaaaatagctgaagagaaaagaaaaagggatgaaataaatatggtaaaggatgccataaaactcacctctgatttctacagaacgatatatgatgagttcggtaaacaagcatccgaacttgctaaggagctggcttctgtatctcaagggaaacagattaagagtgtggatgatgcactgaacgcttttgataaattccgtaataatctgaacaagaaatataacatacaagatcgcatggccatttctaaagccctggaagctattaatcaggtccatatggcggagaattttaagctgttcagtaaggcatttggttttaccggaaaagttattgaacgttatgatgttgctgtggagttacaaaaggctgtaaaaacggacaactggcgtccattttttgtaaaacttgaatcactggcagcaggaagagctgcttcagcagttacagcatgggcgttttccgtcatgctgggaacccctgtaggtattctgggttttgcaattattatggcggctgtgagtgcgcttgttaatgataagtttattgagcaggtcaataaacttattggtatctga

SEQ ID NO: 60 - protein, 271 aa, Escherichia coli
METLTVHAPSPSTNLPSYGNGAFSLSAPHVPGAGPLLVQVVYSFFQSPNMCLQALTQLEDYIKKHGASNPLTLQIISTNIGYFCNADRNLVLHPGISVYDAYHFAKPAPSQYDYRSMNMKQMSGNVTTPIVALAHYLWGNGAERSVNIANIGLKISPMKINQIKDIIKSGVVGTFPVSTKFTHATGDYNVITGAYLGNITLKTEGTLTISANGSWTYNGVVRSYDDKYDFNASTHRGIIGESLTRLGAMFSGKEYQILLPGEIHIKESGKR

SEQ ID NO: 61 - DNA, 816 bp, Escherichia coli
  source 1..816: mol_type = genomic DNA
atggaaaccttaactgttcatgcaccatcaccatcaactaacttaccaagttatggcaatggtgcattttctctttcagcaccacatgtgcctggtgctggccctcttttagtccaggttgtttatagttttttccagagtccaaacatgtgtcttcaggctttaactcaacttgaggattacatcaaaaaacatggggccagcaaccctctcacattgcagatcatatcgacaaatattggttacttctgtaacgccgaccgaaatctggttcttcaccctggaataagcgtttatgacgcttaccacttcgcaaaaccagcgccaagtcaatatgactatcgctcaatgaatatgaaacaaatgagcggtaatgtcactacaccaattgtggcgcttgctcactatttatggggtaatggcgctgaaaggagcgttaatatcgccaacattggtcttaaaatttcccctatgaaaattaatcagataaaagacattataaaatctggtgtagtaggcacattccctgtttctacaaagttcacacatgccactggtgattataatgttattaccggtgcatatcttggtaatatcacactgaaaacagaaggtactttaactatctctgccaatggctcctggacttacaatggcgttgttcgttcatatgatgataaatacgattttaacgccagcactcaccgtggcattatcggagagtcgctcacaaggctcggggcgatgttttctggtaaagagtaccagatactgcttcctggtgaaattcacattaaagaaagtggtaagcgataa

SEQ ID NO: 62 - protein, 387 aa, Escherichia coli
MGSNGADNAHNNAFGGGKNPGIGNTSGAGSNGSASSNRGNSNGWSWSNKPHKNDGFHSDGSYHITFHGDNNSKPKPGGNSGNRGNNGDGASAKVGEITITPDNSKPGRYISSNPEYSLLAKLIDAESIKGTEVYTFHTRKGQYVKVTVPDSNIDKMRVDYVNWKGPKYNNKLVKRFVSQFLLFRKEEKEKNEKEALLKASELVSGMGDKLGEYLGVKYKNVAKEVANDIKNFHGRNIRSYNEAMASLNKVLANPKMKVNKSDKDAIVNAWKQVNAKDMANKIGNLGKAFKVADLAIKVEKIREKSIEGYNTGNWGPLLLEVESWIIGGVVAGVAISLFGAVLSFLPISGLAVTALGVIGIMTISYLSSFIDANRVSNINNIISSVIR

SEQ ID NO: 63 - DNA, 51 bp, Escherichia coli
  source 1..51: mol_type = genomic DNA
gcaaatcgagtttcgaatataaataacattatatctagtgttattcgatga

SEQ ID NO: 64 - protein, 103 aa, Escherichia coli
MRTLTLNELDSVSGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 65 - DNA, 312 bp, Escherichia coli
  source 1..312: mol_type = genomic DNA
atgagaactctgactctaaatgaattagattctgtttctggtggtgcttcagggcgtgatattgcgatggctataggaacactatccggacaatttgttgcaggaggaattggagcagctgctgggggtgtggctggaggtgcaatatatgactatgcatccactcacaaacctaatcctgcaatgtctccatccggtttaggaggaacaattaagcaaaaacccgaagggataccttcagaagcatggaactatgctgcgggaagattgtgtaattggagtccaaataatcttagtgatgtttgtttataa

SEQ ID NO: 66 - protein, 58 aa, Enterococcus columbae
MMNATENQIFVETVSDQELEMLIGGAGRGWIKTLTKDCPNVISSICAGTIITACKNCA

SEQ ID NO: 67 - DNA, 177 bp, Enterococcus columbae
  source 1..177: mol_type = genomic DNA
atgatgaatgctactgaaaaccaaatttttgttgagactgtgagtgaccaagaattagaaatgttaattggtggtgcaggtcgtggatggattaagactttaacaaaagattgtccaaatgtgatttcttcaatttgtgcaggtacaattattacagcttgtaaaaattgtgcttaa

SEQ ID NO: 68 - protein, 59 aa, Lactobacillus curvatus
MNNVKELSMTELQTITGGARSYGNGVYCNNKKCWVNRGEATQSIIGGMISGWASGLAGM

SEQ ID NO: 69 - DNA, 180 bp, Lactobacillus curvatus
  source 1..180: mol_type = genomic DNA
atgaataatgtaaaagaattaagtatgacagaattacaaacaattaccggcggtgctagatcatatggcaacggtgtttactgtaataataaaaaatgttgggtaaatcggggtgaagcaacgcaaagtattattggtggtatgattagcggctgggctagtggtttagctggaatgtaa

SEQ ID NO: 70 - protein, 64 aa, Streptomyces sp.
MRSEMTLTSTNSAEALAAQDFANTVLSAAAPGFHADCETPAMATPATPTVAQFVIQGSTICLVC

SEQ ID NO: 71 - DNA, 195 bp, Streptomyces sp.
  source 1..195: mol_type = genomic DNA
gtgcgatctgagatgactcttacgagcacgaattccgctgaggctctggcggcgcaggactttgcgaacaccgttctcagcgcggcggccccgggcttccacgcggactgcgagacgccggccatggccaccccggccacgccgaccgtcgcccagttcgtgatccagggcagcacgatctgcctggtctgctga

SEQ ID NO: 72 - protein, 65 aa, Bacillus halodurans
MVNSKDLRNPEFRKAQGLQFVDEVNEKELSSLAGSGDVHAQTTWPCATVGVSVALCPTTKCTSQC

SEQ ID NO: 73 - DNA, 198 bp, Bacillus halodurans
  source 1..198: mol_type = genomic DNA
atggtaaattcaaaagatttgcgtaatcctgaattccgcaaagcccaaggtctacaattcgttgacgaggtgaacgagaaggaactttcgtctctagctggttcaggagatgtgcatgcacaaacaacttggccttgcgctacagttggtgtctccgtagccttgtgcccaactacaaagtgtacaagccagtgctaa

SEQ ID NO: 74 - protein, 66 aa, Carnobacterium divergens
MKNLKEGSYTAVNTDELKSINGGTKYYGNGVYCNSKKCWVDWGQASGCIGQTVVGGWLGGAIPGKC

SEQ ID NO: 75 - DNA, 201 bp, Carnobacterium divergens
  source 1..201: mol_type = genomic DNA
atgaaaaacttaaaagaaggttcatacactgctgttaatactgatgaattaaaaagtatcaatggtggaacaaaatattatgggaatggcgtttattgcaattctaaaaaatgttgggtagattggggacaagcttcaggttgtatcggtcaaactgttgttggcggatggctaggcggagctataccaggtaaatgctaa

SEQ ID NO: 76 - protein, 63 aa, Carnobacterium divergens
MIKREKNRTISSLGYEEISNHKLQEIQGGKGILGKLGVVQAGVDFVSGVWAGIKQSAKDHPNA

SEQ ID NO: 77 - DNA, 192 bp, Carnobacterium divergens
  source 1..192: mol_type = genomic DNA
atgattaaaagagaaaagaacagaacaatttcttcccttggttatgaagaaatttctaatcataaattgcaagaaatacaaggtggaaaaggaattcttggtaaactaggagtagtacaggcaggagtggattttgtatcaggagtgtgggctggaataaaacagtctgccaaagatcatcctaatgcgtaa

SEQ ID NO: 78 - protein, 75 aa, Carnobacterium divergens
MKKQILKGLVIVVCLSGATFFSTPQQASAAAPKITQKQKNCVNGQLGGMLAGALGGPGGVVLGGIGGAIAGGCFN

SEQ ID NO: 79 - DNA, 228 bp, Carnobacterium divergens
  source 1..228: mol_type = genomic DNA
atgaaaaaacaaattttaaaagggttggttatagttgtttgtttatctggggcaacatttttctcaacaccacaacaagcttctgctgctgcaccgaaaattactcaaaaacaaaaaaattgtgttaatggacaattaggtggaatgcttgctggagctttgggtggacctggcggagttgtgttaggtggtataggtggtgcaatagcaggaggttgttttaattaa

SEQ ID NO: 80 - protein, 72 aa, Enterococcus durans
MQTIKELNTMELQEIIGGENDHRMPYELNRPNNLSKGGAKCAAGILGAGLGAVGGGPGGFISAGISAVLGCM

SEQ ID NO: 81 - DNA, 219 bp, Enterococcus durans
  source 1..219: mol_type = genomic DNA
atgcaaacgatcaaagaattgaacacgatggaattacaagaaataattggaggtgaaaatgaccatcggatgccttacgaattgaaccgtccaaataatttatccaaaggtggggctaagtgtgctgctggaatacttggcgctggactaggcgcagtaggcggtggacctggcggatttattagtgccggaatcagtgctgttcttggttgtatgtaa

SEQ ID NO: 82 - protein, 72 aa, Enterococcus durans
MQTIKELNTMELQKIIGGENDHRMPYELNRPNNLSKGGAKCAAGILGAGLGAVGGGPGGFISAGISAVLGCM

SEQ ID NO: 83 - DNA, 219 bp, Enterococcus durans
  source 1..219: mol_type = genomic DNA
atgcaaacgatcaaagaattgaacacgatggaattacaaaaaataattggaggtgaaaatgaccatcggatgccttacgaattgaaccgtccaaataatttatccaaaggtggagctaagtgcgctgccggaatacttggtgctggattaggcgcagtaggcggtggacctggcggatttattagtgccggaatcagtgctgttcttggttgtatgtaa

SEQ ID NO: 84 - protein, 220 aa, Streptococcus dysgalactiae
  source 1..220: note = subsp. equisimilis
MKKLKRLVISLVTSLLVISSTVPALVYANETNNFAETQKEITTNSEATLTNEDYTKLTSEVKTIYTNLIQYDQTKNKFYVDEDKTEQYYNYDDESIKGVYLMKDSLNDELNNNNSSNYSEIINQKISEIDYVLQGNDINNLIPSNTRVKRSADFSWIQRCLEEAWGYAISLVTLKGIINLFKAGKFEAAAAKLASATAGRIAGMAALFAFVATCGATTVS

SEQ ID NO: 85 - DNA, 663 bp, Streptococcus dysgalactiae
  source 1..663: mol_type = genomic DNA; note = subsp. equisimilis
atgaaaaaattaaaacgtcttgttatctctcttgttacttcattactagtaatttcaagtacagttccagcacttgtttacgctaatgaaacaaataactttgcagaaactcaaaaagaaattacaacaaattcagaagcaacattaaccaatgaagactacactaaattaacttccgaagtaaaaacaatttatacaaatctgattcaatacgaccaaacaaaaaacaaattttacgtcgatgaagacaaaactgaacaatattataactacgatgatgaaagtataaaaggggtttatctcatgaaagatagtttgaacgatgagttaaacaataataactcttcaaactattctgaaataattaatcaaaaaatctctgaaattgactatgtccttcaaggaaacgatataaataatttaattcctagcaataccagagtaaaaagatcagcagatttttcttggattcaaagatgtctagaagaagcatggggatatgctattagtctagttactctaaaaggaataatcaatctatttaaagcaggaaaatttgaagctgctgctgctaaattagcttctgctacagcaggtagaatcgctggaatggctgccttatttgctttcgtagcaacttgcggtgcgacaactgtatcataa

SEQ ID NO: 86 - protein, 57 aa, Enterococcus faecalis
MKQYKVLNEKEMKKPIGGESVFSKIGNAVGPAAYWILKGLGNMSDVNQADRINRKKH

SEQ ID NO: 87 - DNA, 174 bp, Enterococcus faecalis
  source 1..174: mol_type = genomic DNA
atgaagcaatataaagtattgaatgaaaaagaaatgaaaaaacctattgggggagagtcggtttttagtaaaataggtaatgctgtaggtccagctgcttattggattttaaaaggattaggtaatatgagtgatgtaaaccaagctgatagaattaatagaaagaaacattaa

SEQ ID NO: 88 - protein, 44 aa, Enterococcus faecalis
MGAIAKLVAKFGWPIVKKYYKQIMQFIGEGWAINKIIDWIKKHI

SEQ ID NO: 89 - DNA, 135 bp, Enterococcus faecalis
  source 1..135: mol_type = genomic DNA
atgggagcaatcgcaaaattagtagcaaagtttggatggccaattgttaaaaagtattacaaacaaattatgcaatttattggagaaggatgggcaattaacaaaattattgattggatcaaaaaacatatttaa

SEQ ID NO: 90 - protein, 43 aa, Enterococcus faecalis
MGAIAKLVAKFGWPFIKKFYKQIMQFIGQGWTIDQIEKWLKRH

SEQ ID NO: 91 - DNA, 132 bp, Enterococcus faecalis
  source 1..132: mol_type = genomic DNA
atgggagcaatcgcaaaattagtagcaaagtttggatggccatttattaaaaaattctacaaacaaattatgcagtttatcggacaaggatggacaatagatcaaattgaaaaatggttaaaaagacattga

SEQ ID NO: 92 - protein, 74 aa, Enterococcus faecalis
MLNKKLLENGVVNAVTIDELDAQFGGMSKRDCNLMKACCAGQAVTYAIHSLLNRLGGDSSDPAGCNDIVRKYCK

SEQ ID NO: 93 - DNA, 225 bp, Enterococcus faecalis
  source 1..225: mol_type = genomic DNA
atgttaaataaaaaattattagaaaatggtgtagtaaatgctgtaacaattgatgaacttgatgctcaatttggtggaatgagcaaacgtgattgtaacttgatgaaggcgtgttgtgctggacaagcagtaacatatgctattcatagtcttttaaatcgattaggtggagactctagtgatccagctggttgtaatgatattgtaagaaaatattgtaaataa

SEQ ID NO: 94 - protein, 65 aa, Enterococcus faecium
MKHLKILSIKETQLIYGGTTHSGKYYGNGVYCTKNKCTVDWAKATTCIAGMSIGGFLGGAIPGKC

SEQ ID NO: 95 - DNA, 195 bp, Enterococcus faecium
  source 1..195: mol_type = genomic DNA
atgaaacatttaaaaattttgtctattaaagagacacaacttatctatgggggtaccactcatagtggaaaatattatggaaatggagtgtattgcactaaaaataaatgtacggtcgattgggccaaggcaactacttgtattgcaggaatgtctataggtggttttttaggtggagcaattccagggaagtgc

SEQ ID NO: 96 - protein, 105 aa, Enterococcus faecalis
MVKENKFSKIFILMALSFLGLALFSASLQFLPIAHMAKEFGIPAAVAGTVLNVVEAGGWVTTIVSILTAVGSGGLSLLAAAGRESIKAYLKKEIKKKGKRAVIAW

SEQ ID NO: 97 - DNA, 318 bp, Enterococcus faecalis
  source 1..318: mol_type = genomic DNA
atggttaaagaaaataaattttctaagatttttattttaatggctttgagttttttggggttagccttgtttagtgcaagtcttcagtttttgcccattgcacatatggctaaagagttcggtataccagcagcagttgcaggaactgtgcttaatgtagttgaagctggtggatgggtcactactattgtatcaattcttactgctgtaggtagcggaggtctttctttactcgctgcagcaggaagagagtcaattaaagcataccttaagaaagaaattaagaaaaaaggaaaaagagcagttattgcttggtaa

SEQ ID NO: 98 - protein, 71 aa, Enterococcus faecium
MQNVKELSTKEMKQIIGGENDHRMPNELNRPNNLSKGGAKCGAAIAGGLFGIPKGPLAWAAGLANVYSKCN

SEQ ID NO: 99 - DNA, 216 bp, Enterococcus faecium
  source 1..216: mol_type = genomic DNA
atgcaaaatgtaaaagaattaagtacgaaagagatgaaacaaattatcggtggagaaaatgatcacagaatgcctaatgagttaaatagacctaacaacttatctaaaggtggagcaaaatgtggtgctgcaattgctgggggattatttggaatcccaaaaggaccactagcatgggctgctgggttagcaaatgtatactctaaatgcaactaa

SEQ ID NO: 100 - protein, 58 aa, Enterococcus mundtii
MKKLTSKEMAQVVGGKYYGNGVSCNKKGCSVDWGKAIGIIGNNSAANLATGGAAGWKS

SEQ ID NO: 101 - DNA, 177 bp, Enterococcus mundtii
  source 1..177: mol_type = genomic DNA
ttgaagaaattaacatcaaaagaaatggcacaagtagtaggtggaaaatactacggtaatggagtctcatgtaataaaaaagggtgcagtgttgattggggaaaagctattggcattattggaaataattctgctgcgaatttagctactggtggagcagctggttggaaaagttaa

SEQ ID NO: 102 - protein, 44 aa, Enterococcus faecalis
MLAKIKAMIKKFPNPYTLAAKLTTYEINWYKQQYGRYPWERPVA

SEQ ID NO: 103 - DNA, 135 bp, Enterococcus faecalis
  source 1..135: mol_type = genomic DNA
atgttagcaaaaattaaagcgatgattaagaagtttccgaacccttatactttagcagctaagctaacgacttacgaaattaattggtataaacaacaatacggtcgttatccttgggagcgccctgtagcataa

SEQ ID NO: 104 - protein, 71 aa, Enterococcus faecium
MRKKLFSLALIGIFGLVVTNFGTKVDAATRSYGNGVYCNNSKCWVNWGEAKENIAGIVISGWASGLAGMGH

SEQ ID NO: 105 - DNA, 216 bp, Enterococcus faecium
  source 1..216: mol_type = genomic DNA
atgagaaaaaaattatttagtttagctcttattggaatatttgggttagttgtgacaaattttggtacaaaagttgatgcagctacgcgttcatatggtaatggtgtttattgtaataatagtaaatgctgggttaactggggagaagctaaagagaatattgcaggaatcgttattagtggctgggcttctggtttggcaggtatgggacattaa

SEQ ID NO: 106 - protein, 34 aa, Enterococcus faecium
MNFLKNGIAKWMTGAELQAYKKKYGCLPWEKISC

SEQ ID NO: 107 - DNA, 105 bp, Enterococcus faecium
  source 1..105: mol_type = genomic DNA
atgaattttcttaaaaatggtatcgcaaaatggatgaccggtgctgaattgcaagcgtataaaaagaaatatggatgcttgccatgggaaaaaatttcttgttaa

SEQ ID NO: 108 - protein, 76 aa, Enterococcus faecalis
MKKKLVKGLVICGMIGIGFTALGTNVEAATYYGNGVYCNKQKCWVDWSRARSEIIDRGVKAYVNGFTKVLGGIGGR

SEQ ID NO: 109 - DNA, 231 bp, Enterococcus faecalis
  source 1..231: mol_type = genomic DNA
atgaaaaagaaattagttaaaggcttagttatttgtggcatgattgggattggttttacagcattaggaacaaatgtagaagccgccacgtattacggaaatggtgtctattgcaataagcaaaaatgttgggtagattggagtagagcacgttctgaaattatagacagaggcgtaaaagcatacgtcaatggatttacgaaagtgttaggtggtataggtggaagataa

SEQ ID NO: 110 - protein, 60 aa, Enterococcus faecalis
MKKEELVGMAKEDFLNVICENDNKLENSGAKCPWWNLSCHLGNDGKICTYSHECTAGCNA

SEQ ID NO: 111 - DNA, 183 bp, Enterococcus faecalis
  source 1..183: mol_type = genomic DNA
atgaaaaaagaagaattagtaggaatggctaaggaagactttttaaatgttatttgtgaaaatgacaacaaactagaaaatagtggagcaaaatgtccttggtggaatctttcttgtcatttaggcaatgatggtaaaatttgcacttattcacatgaatgtaccgcaggttgtaatgcataa

SEQ ID NO: 112 - protein, 61 aa, Enterococcus faecalis
MTELNKRLQLKRDVSTENSLKKISNTDETHGGVTTSIPCTVMVSAAVCPTLVCSNKCGGRG

SEQ ID NO: 113 - DNA, 186 bp, Enterococcus faecalis
  source 1..186: mol_type = genomic DNA
atgactgaacttaacaaaagattacaattaaaaagagatgtttcaacagaaaatagtttgaaaaaaatttctaatactgatgaaacacatgggggagttactacatcaattccatgtacagtaatggttagtgcggcagtatgtcctacccttgtttgctcgaataaatgtggcggtagaggctag

SEQ ID NO: 114 - protein, 58 aa, Enterococcus faecium
MQNVKEVSVKEMKQIIGGSNDSLWYGVGQFMGKQANCITNHPVKHMIIPGYCLSKILG

SEQ ID NO: 115 - DNA, 177 bp, Enterococcus faecium
  source 1..177: mol_type = genomic DNA
atgcaaaatgtaaaagaagtttctgtaaaagagatgaaacaaattatcggtggttctaatgatagtctttggtatggtgtaggacaatttatgggtaaacaagcaaactgtataacaaaccatcctgttaaacacatgataattcctggatattgtttatcgaaaattttagggtaa

SEQ ID NO: 116 - protein, 55 aa, Enterococcus faecium
MKKYNELSKKELLQIQGGIAPIIVAGLGYLVKDAWDHSDQIISGFKKGWNGGRRK

SEQ ID NO: 117 - DNA, 168 bp, Enterococcus faecium
  source 1..168: mol_type = genomic DNA
atgaaaaaatataatgagttatctaaaaaagaacttctacagattcaaggaggaatagcacctattatagttgctggccttggctatttagtaaaagatgcatgggatcactcagatcaaataatctcaggatttaaaaaaggttggaatggtggacgtagaaaataa

SEQ ID NO: 118 - protein, 343 aa, Enterococcus faecalis
MKNILLSILGVLSIVVSLAFSSYSVNAASNEWSWPLGKPYAGRYEEGQQFGNTAFNRGGTYFHDGFDFGSAIYGNGSVYAVHDGKILYAGWDPVGGGSLGAFIVLQAGNTNVIYQEFSRNVGDIKVSTGQTVKKGQLIGKFTSSHLHLGMTKKEWRSAHSSWNKDDGTWFNPIPILQGGSTPTPPNPGPKNFTTNVRYGLRVLGGSWLPEVTNFNNTNDGFAGYPNRQHDMLYIKVDKGQMKYRVHTAQSGWLPWVSKGDKSDTVNGAAGMPGQAIDGVQLNYITPKGEKLSQAYYRSQTTKRSGWLKVSADNGSIPGLDSYAGIFGEPLDRLQIGISQSNPF

SEQ ID NO: 119 - DNA, 1032 bp, Enterococcus faecalis
  source 1..1032: mol_type = genomic DNA
atgaaaaatattttactttctattctaggggtattatctatcgttgtttctttggcgttttcttcttattctgtcaacgcagcttctaatgagtggtcgtggccactgggcaaaccatatgcgggaagatatgaagaaggacaacaattcgggaacactgcatttaaccgaggaggtacttatttccatgatgggtttgactttggttctgctatttatggaaatggcagtgtgtatgctgtgcatgatggtaaaattttatatgctggttgggatcctgtaggtggaggctcattaggtgcatttattgtactacaagcgggaaacacaaatgtgatttatcaagaatttagccgaaatgttggagatattaaagttagcactggacaaactgttaaaaaaggacagctgataggaaagtttacttctagtcatttacatttaggaatgacaaaaaaagaatggcgttctgctcattcttcttggaataaagatgatggcacttggtttaacccaattcctatacttcaaggaggatctacgcctacgcctccaaatccaggaccaaaaaatttcacaacaaatgttcgttacggattgcgggtcctcggaggttcatggttaccagaagtaaccaactttaacaataccaatgatggtttcgcaggttaccctaatcgtcaacatgatatgctttatataaaggtagataaagggcaaatgaaatatcgtgttcacacggctcaaagtggatggttgccttgggtaagtaaaggggataagagcgatacagtaaatggagcggcaggtatgcctggacaagcaattgatggtgttcagctaaactatataactcctaagggagaaaaattatcacaggcttactatcgttcacaaactacgaaacgatcaggctggttaaaagtaagtgcagataatggttctattcctggactagacagttatgcaggaatctttggagaaccgttggatcgcttgcaaataggtatttcacagtcaaatccattttaa

SEQ ID NO: 120 - protein, 56 aa, Staphylococcus epidermidis
MENKKDLFDLEIKKDNMENNNELEAQSLGPAIKATRQVCPKATRFVTVSCKKSDCQ

SEQ ID NO: 121 - DNA, 171 bp, Staphylococcus epidermidis
  source 1..171: mol_type = genomic DNA
atggaaaacaaaaaagatttatttgatttagaaatcaaaaaagataatatggaaaataataatgaattagaagctcaatctcttggtcctgcaattaaggcaactagacaggtatgtcctaaagcaacacgttttgttacagtttcttgtaaaaaaagtgattgtcaatag

SEQ ID NO: 122 - protein, 51 aa, Staphylococcus epidermidis
MAAFMKLIQFLATKGQKYVSLAWKHKGTILKWINAGQSFEWIYKQIKKLWA

SEQ ID NO: 123 - DNA, 156 bp, Staphylococcus epidermidis
  source 1..156: mol_type = genomic DNA
atggcagcatttatgaagttaattcagttcttagcaactaaaggtcaaaagtatgtttcacttgcatggaaacataaaggtactattttaaaatggattaacgccggtcaaagttttgaatggatttataaacaaatcaaaaaattatgggcataa

SEQ ID NO: 124 - protein, 52 aa, Staphylococcus epidermidis
MEAVKEKNDLFNLDVKVNAKESNDSGAEPRIASKFICTPGCAKTGSFNSYCC

SEQ ID NO: 125 - DNA, 159 bp, Staphylococcus epidermidis
  source 1..159: mol_type = genomic DNA
atggaagcagtaaaagaaaaaaatgatctttttaatcttgatgttaaagttaatgcaaaagaatctaacgattcaggagctgaaccaagaattgctagtaaatttatatgtactcctggatgtgcaaaaacaggtagttttaacagttattgttgttaa

SEQ ID NO: 126 - protein, 55 aa, Staphylococcus epidermidis
MNNSLFDLNLNKGVETQKSDLSPQSASVLKTSIKVSKKYCKGVTLTCGCNITGGK

SEQ ID NO: 127 - DNA, 168 bp, Staphylococcus epidermidis
  source 1..168: mol_type = genomic DNA
atgaataactcattattcgatttaaacctaaacaaaggtgtagaaactcaaaagagtgatttaagtccgcaatctgctagtgtcttgaagacttctattaaagtatctaaaaaatattgtaaaggtgttactttaacatgcggttgcaatattactggtggtaaataa

SEQ ID NO: 128 - protein, 52 aa, Staphylococcus gallinarum
MEAVKEKNELFDLDVKVNAKESNDSGAEPRIASKFLCTPGCAKTGSFNSYCC

SEQ ID NO: 129 - DNA, 159 bp, Staphylococcus gallinarum
  source 1..159: mol_type = genomic DNA
atggaagcagtaaaagagaaaaatgaactttttgatcttgacgttaaagtaaatgcaaaagagtctaatgattcaggcgcagaaccacgaattgctagtaaatttttatgtactcctggatgtgccaaaacaggtagcttcaatagctactgttgttaa

SEQ ID NO: 130 - protein, 63 aa, Lactococcus garvieae
MENNNYTVLSDEELQKIDGGIGGALGNALNGLGTWANMMNGGGFVNQWQVYANKGKINQYRPY

SEQ ID NO: 131 - DNA, 192 bp, Lactococcus garvieae
  source 1..192: mol_type = genomic DNA
atggaaaacaacaattacacagtactttcagatgaagaactacaaaaaattgatggtggaatcggcggggctcttggtaatgctctcaacggattaggtacctgggcaaacatgatgaacggtggaggatttgttaatcagtggcaagtttatgctaataaaggaaaaataaatcaataccgtccgtattaa

SEQ ID NO: 132 - protein, 63 aa, Lactococcus garvieae
MFDLVATGMAAGVAKTIVNAVSAGMDIATALSLFSGAFTAAGGIMALIKKYAQKKLWKQLIAA

SEQ ID NO: 133 - DNA, 192 bp, Lactococcus garvieae
  source 1..192: mol_type = genomic DNA
atgtttgatttagtcgcgactggaatggctgcaggtgtagcaaaaactattgttaatgccgttagtgctggtatggatattgccactgctttatcattgttctcaggagcttttactgcagctgggggaattatggcactcattaaaaaatatgctcaaaagaaattatggaaacagcttattgctgcataa

SEQ ID NO: 134 - protein, 91 aa, Lactobacillus gasseri
MVTKYGRNLGLNKVELFAIWAVLVVALLLTTANIYWIADQFGIHLATGTARKLLDAMASGASLGTAFAAILGVTLPAWALAAAGALGATAA

SEQ ID NO: 135 - DNA, 276 bp, Lactobacillus gasseri
  source 1..276: mol_type = genomic DNA
atggttactaagtacggacgtaatttaggtttgaacaaggtagagttgtttgcaatttgggcggttttagtagttgctcttttattgaccacagcgaacatttattggattgctgatcaattcgggattcatttagcgactggaacagcccgtaagttattagatgcaatggcttctggtgcctcattgggaactgcctttgctgctattttgggcgtgacattacctgcatgggctttggcagctgcaggagcattgggagcgactgcagcctag

SEQ ID NO: 136 - protein, 75 aa, Lactobacillus gasseri
MKNFNTLSFETLANIVGGRNNWAANIGGVGGATVAGWALGNAVCGPACGFVGAHYVPIAWAGVTAATGGFGKIRK

SEQ ID NO: 137 - DNA, 228 bp, Lactobacillus gasseri
  source 1..228: mol_type = genomic DNA
atgaaaaattttaatacattatcatttgaaacattggctaacatagttggtgggagaaataattgggctgctaatataggtggagtaggtggagcgacagtcgctggatgggctcttggaaatgcagtttgcggtcctgcttgtggctttgttggagcacactatgttccaatagcatgggctggcgtaacggcagctactggtggattcggaaagataagaaagtag

SEQ ID NO: 138 - protein, 64 aa, Lactobacillus plantarum
MSKLVKTLTISEISKAQNNGGKPAWCWYTLAMCGAGYDSGTCDYMYSHCFGIKHHSSGSSSYHC

SEQ ID NO: 139 - DNA, 195 bp, Lactobacillus plantarum
  source 1..195: mol_type = genomic DNA
atgagtaaattggttaagacacttactataagtgaaatttctaaggctcaaaacaacggtggaaaacctgcatggtgttggtatactttagcaatgtgtggtgctggttatgattcgggaacctgtgattatatgtattcgcattgttttggtataaagcatcatagtagtggtagtagcagttatcattgttag

SEQ ID NO: 140 - protein, 359 aa, Haloferax mediterranei
MSKDRDGRRTSRRGTLKKIGGFSLGALSFGAVGRTQAATGSSVTTADIAPPGPNGDPKSVQIDDKYTGAEMYGEGDFRVGLGTDLTMYPPVYRESLGNGSGGWEFDFTVCGSTACRFVDSNGDVKEDDKAKEMWWQEINFNDINQDLYSRNDSDWVGSTPADTQPEFDYTEFALARDGVTLALTALNPAMGSLALGATYFLSDMVNWIASQHEDDSSLKRKWDYDGLSGPLYADSSTYLLARDEMTSNSYESFTIDNIAVAFPEFPVRTKYYVTFTAPDDPSTQSISTLEEEGIYRVPATEVAAARPPGSRRSKSAADEMVYVADPKKFIEVEPVKNPSIPDRIYEEIEQKKKQRSRKQ

SEQ ID NO: 141 - DNA, 1080 bp, Haloferax mediterranei
  source 1..1080: mol_type = genomic DNA
atgtcgaaagacagagatgggagaaggacaagtcggcgaggcacgttaaagaaaatcggcggtttcagtctcggagcgcttagtttcggggcagtcggacgaactcaagcggcgaccggctcatcggttacgaccgctgatatcgcacctcccggaccgaacggagacccgaagagtgttcagatagatgataaatacaccggagccgagatgtacggcgagggtgacttcagagtcggtctcggaactgacctgacgatgtatccgcccgtgtaccgtgagagtcttggaaatggaagcgggggttgggaattcgacttcaccgtttgtgggtccactgcctgtcgatttgtggacagtaacggtgacgtcaaagaggacgacaaggcgaaagaaatgtggtggcaggaaattaacttcaacgacataaatcaggatttatacagtcggaacgattccgactgggtcgggtcgacccctgccgatacccaaccggagttcgattacaccgactttgcgctcgctcgggacggagtgacgctcgctctcacggcactcaaccccgcaatggggagtcttgcactcggtgccacgtacttcctcagcgacatggtgaactggattgcgagccagcacgaagacgacagttcgctcaagagaaaatgggattacgacgggctaagtgggccgttgtacgccgattcgtcgacgtacctactggcacgcgacgagatgacttcgaactcgtacgaatcattcacgatcgataacatcgccgttgccttcccagagttccccgtccggaccaagtactacgtcacattcactgcgccggatgacccgtcaacgcagtcgatatctacgctcgaagaggagggaatctaccgagtgcccgctacggaagtggctgcggccagaccaccggggtcccgacgttccaaatcggcagccgacgagatggtgtacgttgccgatccgaagaagttcatagaggtcgagccggtgaagaacccaagtatcccggaccgaatctacgaggagatagagcaaaaaaagaaacaacggagtaggaaacagtag

SEQ ID NO: 142 - protein, 311 aa, unidentified
  source 1..311: note = S8a; note = Haloarchaeon species
MSDKDSINRRNVLRKIGGIGVASAVGFSGLASGESLSDDEKQDVIDTIYKSQRVEQIKKKFGGVNIEPKKVQSVTTNQSGDLVTAKLSVSDGDLVYSSVKDTTVIVQFDRSASEIGESWPKNTEAFIKSTSSGVDLLRTATDEEIKDVTEGVNTSEIESADAVNIFIDPESQTYYMEKYDFNNKVLEMFELATGGTSSGKISPTREDQNHEYNVREHKVFNSEKQNIQLQSDCNINSNTAADVILCFNQVGSCALCSPTLVGGPVPTVACLLVVCFGTPNAVSAILEEVDNSCFNLIKDVISCWDEWTSFW

SEQ ID NO: 143 - DNA, 936 bp, unidentified
  source 1..936: mol_type = genomic DNA; note = S8a; note = Haloarchaeon species
atgtcggataaagacagcattaacagaagaaatgtattaagaaaaattggcggtatcggtgtggcttcagctgtcggattttctggtttggcaagcggggaaagtcttagcgatgatgagaaacaagatgttattgacacaatttacaaatcacaaagagttgaacagataaagaaaaagttcggaggagtgaatattgagccgaaaaaggttcaatctgtaacgaccaatcagagcggagatcttgttacggcgaagctgtcggttagtgatggggatttggtatattcgagtgtcaaagatacaactgtaatagttcagttcgatagatcggcttctgaaattggtgaaagttggcccaagaatactgaggcattcatcaaatcgacgtcctctggggtcgatcttctacgtacagcaactgatgaagaaataaaggacgttactgagggagtcaacacatctgaaattgaatctgcggatgctgttaacatatttattgatcctgaatcacagacatactatatggagaaatatgactttaataataaggtacttgagatgtttgaattagcgacaggtgggacaagtagtggtaaaatctcccccacacgtgaagaccagaatcacgaatataatgttagggaacataaagtatttaactcagaaaaacagaatatacaacttcagagtgactgtaatataaacagtaacaccgctgctgatgttattctatgcttcaaccaggttggttcttgtgcactctgctccccgactttagtcggaggtccagtccctacagttgcatgtctcttagtcgtctgtttcggcactccaaatgctgtgtccgcgatacttgaagaagtcgataattcttgctttaacttgatcaaggatgtaatttcgtgttgggatgaatggactagcttctggtga

SEQ ID NO: 144 - protein, 333 aa, Lactobacillus helveticus
MKHLNETTNVRILSQFDMDTGYQAVVQKGNVGSKYVYGLQLRKGATTILRGYRGSKINNPILELSGQAGGHTQTWEFAGDRKDINGEERAGQWFIGVKPSKIEGSKIIWAKQIARVDLRNQMGPHYSNTDFPRLSYLNRAGSNPFAGNKMTHAEAAVSPDYTKFLIATVENNCIGHFTIYNLDTINEKLDEKGNSEDVNLETVKYEDSFIIDNLYGDDNNSIVNSIQGYDLDNDGNIYISSQKAPDFDGSYYAHHKQIVKIPYYARSKESEDQWRAVNLSEFGGLDIPGKHSEVESIQIIGENHCYLTVAYHSKNKAGENKTTLNEIYELSWN

SEQ ID NO: 145 - DNA, 1002 bp, Lactobacillus helveticus
  source 1..1002: mol_type = genomic DNA
atgaagcatttaaatgaaacaactaatgttagaattttaagtcaatttgatatggatactggctatcaagcagtagttcaaaaaggcaatgtaggttcaaaatatgtatatggattacaacttcgcaaaggtgctactactatcttgcgtggttaccgtggaagtaaaattaataaccctattcttgaattatctggtcaagcaggtggtcacacacagacatgggaatttgctggtgatcgtaaagacattaatggtgaagaaagagcaggtcaatggtttataggtgttaaaccatcgaaaattgaaggaagcaaaattatttgggcaaagcaaattgcaagagttgatcttagaaatcaaatgggacctcattattcaaatactgactttcctcgattatcctacttgaatcgcgccggttctaatccatttgctggtaataagatgacgcatgccgaagccgcagtatcacctgattatactaagtttttaattgctactgttgaaaataactgtattggtcattttactatatacaatttagatacaattaatgaaaaacttgatgaaaagggaaatagtgaagatgttaatctcgaaactgttaaatacgaagatagttttatcattgataatttatatggtgatgataataattctattgtaaattcaattcaagggtatgatttggataatgatggaaatatttatatttccagtcaaaaagcgccagattttgatggctcttattatgcacatcataagcagattgttaagattccatattatgctcggtctaaagaaagcgaagaccaatggagagctgtaaatttaagcgaattcggtggcttggatattccaggtaaacatagtgaagttgaaagcatccaaattattggtgagaatcattgttacttaactgttgcatatcattctaaaaataaagcgggtgaaaataaaactactttgaatgagatttatgaattatcttggaattag

SEQ ID NO: 146 - protein, 74 aa, Enterococcus hirae; identical to SEQ ID NO: 20

SEQ ID NO: 147 - DNA, 225 bp, Enterococcus hirae; identical to SEQ ID NO: 21
  source 1..225: mol_type = genomic DNA

SEQ ID NO: 148 - protein, 75 aa, Lactobacillus johnsonii
MKQFNYLSHKDLAVVVGGRNNWQTNVGGAVGSAMIGATVGGTICGPACAVAGAHYLPILWTAVTAATGGFGKIRK

SEQ ID NO: 149 - DNA, 228 bp, Lactobacillus johnsonii
  source 1..228: mol_type = genomic DNA
atgaaacaatttaattatttatcacataaagatttagcagtcgttgttggtggaagaaataattggcaaacaaatgtgggaggagcagtgggatcagctatgattggggctacagttggtggtacaatttgtggacctgcatgtgctgtagctggtgcccattatcttcctattttatggacagcggttacagctgcaacaggtggttttggcaagataagaaagtag

SEQ ID NO: 150 - protein, 62 aa, Lactobacillus johnsonii
MKLNDKELSKIVGGNRWGDTVLSAASGAGTGIKACKSFGPWGMAICGVGGAAIGGYFGYTHN

SEQ ID NO: 151 - DNA, 189 bp, Lactobacillus johnsonii
  source 1..189: mol_type = genomic DNA
atgaaattaaatgacaaagaattatcaaagattgttggtggaaatcgatggggagatactgttttatcagctgctagtggcgcaggaactggtattaaagcatgtaaaagttttggcccatggggaatggcaatttgtggtgtaggaggtgcagcaataggaggttattttggctatactcataattaa

SEQ ID NO: 152 - protein, 59 aa, Lactococcus lactis
  source 1..59: note = subsp. lactis
MNKNEIETQPVTWLEEVSDQNFDEDVFGACSTNTFSLSDYWGNNGAWCTLTHECMAWCK

SEQ ID NO: 153 - DNA, 180 bp, Lactococcus lactis
  source 1..180: mol_type = genomic DNA; note = subsp. lactis
atgaacaaaaatgaaattgaaacacaaccagttacatggttggaagaagtatctgatcaaaattttgatgaagatgtatttggtgcgtgtagtactaacacattctcgctcagtgattactggggaaataacggggcttggtgtacactcactcatgaatgtatggcttggtgtaaataa

SEQ ID NO: 154 - protein, 65 aa, Lactococcus lactis
  source 1..65: note = subsp. lactis
MKEKNMKKNDTIELQLGKYLEDDMIELAEGDESHGGTTPATPAISILSAYISTNTCPTTKCTRAC

SEQ ID NO: 155 - DNA, 198 bp, Lactococcus lactis
  source 1..198: mol_type = genomic DNA; note = subsp. lactis
atgaaagaaaaaaatatgaaaaagaatgacactattgaattacaattgggaaaataccttgaagatgatatgattgaattagctgaaggggatgagtctcatggaggaacaacaccagcaactcctgcaatctctattctcagtgcatatattagtaccaatacttgtccaacaacaaaatgtacacgtgcttgttaa

SEQ ID NO: 156 - protein, 51 aa, Lactococcus lactis
  source 1..51: note = subsp. lactis
MKEQNSFNLLQEVTESELDLILGAKGGSGVIHTISHECNMNSWQFVFTCCS

SEQ ID NO: 157 - DNA, 156 bp, Lactococcus lactis
  source 1..156: mol_type = genomic DNA; note = subsp. lactis
atgaaagaacaaaactcttttaatcttcttcaagaagtgacagaaagtgaattggaccttattttaggtgcaaaaggcggcagtggagttattcatacaatttctcatgaatgtaatatgaatagctggcaatttgtatttacttgctgctcttaa

SEQ ID NO: 158 - protein, 53 aa, Lactococcus lactis
  source 1..53: note = subsp. lactis
MAGFLKVVQLLAKYGSKAVQWAWANKGKILDWLNAGQAIDWVVSKIKQILGIK

SEQ ID NO: 159 - DNA, 162 bp, Lactococcus lactis
  source 1..162: mol_type = genomic DNA; note = subsp. lactis
atggcagggtttttaaaagtagttcaattactagctaaatatggttctaaagctgtacaatgggcttgggcaaacaagggtaagattttagattggcttaatgcaggtcaggctattgattgggtagtttcgaaaattaagcaaattttaggtattaagtaa

SEQ ID NO: 160 - protein, 53 aa, Lactococcus lactis
  source 1..53: note = subsp. lactis
MAGFLKVVQILAKYGSKAVQWAWANKGKILDWINAGQAIDWVVEKIKQILGIK

SEQ ID NO: 161 - DNA, 162 bp, Lactococcus lactis
  source 1..162: mol_type = genomic DNA; note = subsp. lactis
atggcagggtttttaaaagtagtccaaattttggctaagtatggttctaaagccgtacaatgggcatgggcaaataaaggaaaaatcttagattggattaatgcaggtcaagctattgactgggtagttgaaaagattaagcaaattttgggtattaaataa

SEQ ID NO: 162 - protein, 65 aa, Lactobacillus amylovorus
MKQLNSEQLQNIIGGNRWTNAYSAALGCAVPGVKYGKKLGGVWGAVIGGVGGAAVCGLAGYVRKG

SEQ ID NO: 163 - DNA, 198 bp, Lactobacillus amylovorus
  source 1..198: mol_type = genomic DNA
atgaaacaattgaattcagaacaattacaaaatattatcggtggaaatagatggactaatgcatacagcgcagctttgggatgcgctgtccctggagttaaatatggaaaaaaacttggtggcgtatggggtgctgtaattggtggcgtaggcggtgcagcagtctgtggcttggcgggttatgttcgtaaaggctaa

SEQ ID NO: 164 - protein, 68 aa, Lactobacillus sakei
  source 1..68: note = L45
MKTEKKVLDELSLHASAKMGARDVESSMNADSTPVLASVAVSMELLPTASVLYSDVAGCFKYSAKHHC

SEQ ID NO: 165 - DNA, 207 bp, Lactobacillus sakei
  source 1..207: mol_type = genomic DNA; note = L45
atgaaaacagaaaaaaaggttttagatgaactgagcttacacgcttctgcaaaaatgggagcacgtgatgttgaatccagcatgaatgcagactcaacaccagttttagcatcagtcgctgtatccatggaattattgccaactgcgtctgttctttattcggatgttgcaggttgcttcaaatattctgcaaaacatcattgttag

SEQ ID NO: 166 - protein, 91 aa, Lactococcus lactis
  source 1..91: note = subsp. lactis
MKTKSLVLALSAVTLFSAGGIVAQAEGTWQHGYGVSSAYSNYHHGSKTHSATVVNNNTGRQGKDTQRAGVWAKATVGRNLTEKASFYYNFW

SEQ ID NO: 167 - DNA, 276 bp, Lactococcus lactis
  source 1..276: mol_type = genomic DNA; note = subsp. lactis
atgaaaaccaagtctctcgtattggcattatctgcggttacgttattctctgccggaggaattgtagctcaagctgaaggaacatggcaacatggatatggtgttagttcggcatattcaaattatcatcatggtagcaaaactcattcagccacagttgtaaataataatactggccgacaaggtaaggatacacaacgtgccggtgtttgggcaaaagctactgttggacgtaacttaactgaaaaagcttcattttattataacttttggtaa

SEQ ID NO: 168 - protein, 75 aa, Lactococcus lactis
  source 1..75: note = subsp. cremoris
MKNQLNFNIVSDEELSEANGGKLTFIQSTAAGDLYYNTNTHKYVYQQTQNAFGAAANTIVNGWMGGAAGGFGLHH

SEQ ID NO: 169 - DNA, 228 bp, Lactococcus lactis
  source 1..228: mol_type = genomic DNA; note = subsp. cremoris
atgaaaaatcaattaaattttaatattgtttcagatgaagaactttcagaagctaacggaggaaaattaacatttattcaatcgacagcggctggagatttatattacaatactaatacacacaaatatgtttaccaacaaactcaaaacgcttttggggctgctgctaataccattgttaatggatggatgggtggcgctgctggaggtttcgggttgcaccattga

SEQ ID NO: 170 - protein, 68 aa, Lactococcus lactis
  source 1..68: note = subsp. cremoris
MKNQLNFNIVSDEELAEVNGGSLQYVMSAGPYTWYKDTRTGKTICKQTIDTASYTFGVMAEGWGKTFH

SEQ ID NO: 171 - DNA, 207 bp, Lactococcus lactis
  source 1..207: mol_type = genomic DNA; note = subsp. cremoris
atgaaaaatcaattaaattttaatattgtttctgatgaagaacttgcagaagttaatggaggaagcttgcagtatgttatgagtgctggaccatatacttggtataaagatactagaacaggaaaaacaatatgtaaacagacaattgacacagcaagttatacatttggtgtaatggcagaaggatggggaaaaacattccactaa

SEQ ID NO: 172 - protein, 63 aa, Lactococcus sp.
  source 1..63: note = QU 12
MKLIDHLGAPRWAVDTILGAIAVGNLASWVLALVPGPGWAVKAGLATAAAIVKHQGKAAAAAW

SEQ ID NO: 173 - DNA, 192 bp, Lactococcus sp.
  source 1..192: mol_type = genomic DNA; note = QU 12
atgaaattaattgatcatttaggtgctccaagatgggccgttgatactattttaggtgcaatcgcagttgggaacttagcaagttgggttctagcgcttgtccctggtccagggtgggcagtaaaagctggtttagcaactgctgctgccatcgttaaacatcaaggtaaagctgccgctgctgcttggtaa

SEQ ID NO: 174 - protein, 50 aa, Brevibacillus sp.
  source 1..50: note = GI-9
MACQCPDAISGWTHTDYQCHGLENKMYRHVYAICMNGTQVYCRTEWGSSC

SEQ ID NO: 175 - DNA, 153 bp, Brevibacillus sp.
  source 1..153: mol_type = genomic DNA; note = GI-9
atggcttgccaatgtccagatgcgatctcaggttggacgcatacagattaccagtgtcacggtttggagaataaaatgtatagacatgtttatgcaatttgcatgaacggtactcaagtatattgcagaacagagtggggtagcagctgctag

SEQ ID NO: 176 - protein, 57 aa, Leuconostoc pseudomesenteroides
MNKEYNSISNFKKITNKDLQNINGGFIGRAIGDFVYFGAKGLRESGKLLNYYYKHKH

SEQ ID NO: 177 - DNA, 174 bp, Leuconostoc pseudomesenteroides
  source 1..174: mol_type = genomic DNA
atgaataaagaatataatagcattagcaattttaaaaaaattactaataaagacttgcaaaacataaatggtggatttattggtagggcaataggtgactttgtgtactttggagcgaagggactaagagaatctggtaaactacttaattattactataagcataagcattga

SEQ ID NO: 178 - protein, 53 aa, Leuconostoc pseudomesenteroides
MKNQLMSFEVISEKELSTVQGGKGLGKLIGIDWLLGQAKDAVKQYKKDYKRWH

SEQ ID NO: 179 - DNA, 162 bp, Leuconostoc pseudomesenteroides
  source 1..162: mol_type = genomic DNA
atgaaaaatcagttaatgtctttcgaagtgatatcagaaaaagaattgtccacggtacaaggtggcaaaggcttaggtaaactcataggaattgattggcttttgggtcaagctaaggacgctgttaaacagtacaagaaggattacaaacgttggcactaa

SEQ ID NO: 180 - protein, 61 aa, Leuconostoc gelidum
MMNMKPTESYEQLDNSALEQVVGGKYYGNGVHCTKSGCSVNWGEAFSAGVHRLANGGNGFW

SEQ ID NO: 181 - DNA, 186 bp, Leuconostoc gelidum
  source 1..186: mol_type = genomic DNA
atgatgaacatgaaacctacggaaagctatgagcaattggataatagtgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 182 - protein, 61 aa, Leuconostoc carnosum
MNNMKSADNYQQLDNNALEQVVGGKYYGNGVHCTKSGCSVNWGEAFSAGVHRLANGGNGFW

SEQ ID NO: 183 - DNA, 186 bp, Leuconostoc carnosum
  source 1..186: mol_type = genomic DNA
atgaataacatgaaatctgcggataattatcagcaattggataataatgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 184 - protein, 63 aa, Leuconostoc mesenteroides
MFLVNQLGISKSLANTILGAIAVGNLASWLLALVPGPGWATKAALATAETIVKHEGKAAAIAW

SEQ ID NO: 185 - DNA, 192 bp, Leuconostoc mesenteroides
  source 1..192: mol_type = genomic DNA
atgttcttggtaaatcagttagggatttcaaaatcgttagctaatactattcttggtgcaattgctgttggtaatttggccagttggttattagctttggttcctggtccgggttgggcaacaaaagcagcacttgcgacagctgaaacaattgtgaagcatgaaggaaaagcagctgctattgcgtggtaa

SEQ ID NO: 186 - protein, 74 aa, Bacillus licheniformis
MSKKEMILSWKNPMYRTESSYHPAGNILKELQEEEQHSIAGGTITLSTCAILSKPLGNNGYLCTVTKECMPSCN

SEQ ID NO: 187 - DNA, 225 bp, Bacillus licheniformis
  source 1..225: mol_type = genomic DNA
atgtcaaaaaaggaaatgattctttcatggaaaaatcctatgtatcgcactgaatcttcttatcatccagcagggaacatccttaaagaactccaggaagaggaacagcacagcatcgccggaggcacaatcacgctcagcacttgtgccatcttgagcaagccgttaggaaataacggatacctgtgtacagtgacaaaagaatgcatgccaagctgtaactaa

SEQ ID NO: 188 - protein, 266 aa, Brevibacterium linens
MNNLYRELAPIPGPAWAEIEEEARRTFKRNIAGRRIVDVAGPTGFETSAVTTGHIRDVQSETSGLQVKQRIVQEYIELRTPFTVTRQAIDDVARGSGDSDWQPVKDAATTIAMAEDRAILHGLDAAGIGGIVPGSSNAAVAIPDAVEDFADAVAQALSVLRTVGVDGPYSLLLSSAEYTKVSESTDHGYPIREHLSRQLGAGEIIWAPALEGALLVSTRGGDYELHLGQDLSIGYYSHDSETVELYLQETFGFLALTDESSVPLSL

SEQ ID NO: 189 - DNA, 801 bp, Brevibacterium linens
  source 1..801: mol_type = genomic DNA
gtgaataacctctatcgcgagcttgcccccatccccggcccggcctgggcggagatcgaggaggaggctcgacggacattcaaacgcaatatcgccggccgccggatcgtcgatgtcgcagggcccacgggcttcgagacctccgcggtgaccactggccacatccgagacgtccagtcggagacgagcggactgcaggttaagcagcgcatcgtgcaggaatacatcgagctgcggaccccattcaccgtgactcggcaggccatcgatgacgtggcccgcgggtccggtgactcggactggcagcccgtcaaggatgcggccacgacgatcgcgatggctgaagatcgggccattctccacgggctcgatgcggccgggatcggcggaatcgttcccggcagctcgaatgccgcagtggccatccccgacgccgtcgaggacttcgcggacgccgtcgcccaggcgctgagtgtgctgcgcacggtgggagtcgacgggccctacagcctgttgctctcctccgcggagtacaccaaggtctccgagtccaccgaccacggctacccgatccgcgagcacctctcccggcagctcggcgccggagagatcatctgggcgcccgcgctcgaaggggcgctgctcgtctccacgcgcgggggtgactacgagctccacctcggccaggacctgtcgatcggttactacagccacgacagcgagaccgtcgaactctatctgcaggagaccttcggattcctcgcgctgaccgacgaatccagtgtgcctttgagcctctga

SEQ ID NO: 190 - protein, 71 aa, Listeria innocua
MKKAALKFIIVIAILGFSFSFFSIQSEAKSYGNGVQCNKKKCWVDWGSAISTIGNNSAANWATGGAAGWKS

SEQ ID NO: 191 - DNA, 216 bp, Listeria innocua
  source 1..216: mol_type = genomic DNA
ttgaagaaggcagcgttaaaatttattattgttattgctattctaggtttcagtttttctttctttagcatacaatctgaagctaaatcttatggaaatggagttcagtgtaataagaaaaaatgttgggtagattggggtagtgctataagtactattggaaataattctgcagcgaattgggctacaggtggagcagctggttggaaaagctga

SEQ ID NO: 192 - protein, 68 aa, Bacillus sp.
MSQEAIIRSWKDPFSRENSTQNPAGNPFSELKEAQMDKLVGAGDMEAACTFTLPGGGGVCTLTSECIC

SEQ ID NO: 193 - DNA, 207 bp, Bacillus sp.
  source 1..207: mol_type = genomic DNA
atgagtcaagaagctatcattcgttcatggaaagatcctttttcccgtgaaaattctacacaaaatccagctggtaacccattcagtgagctgaaagaagcacaaatggataagttagtaggtgcgggagacatggaagcagcatgtacttttacattgcctggtggcggcggtgtttgtactctaacttctgaatgtatttgttaa

SEQ ID NO: 194 - protein, 61 aa, Leuconostoc mesenteroides
MTNMKSVEAYQQLDNQNLKKVVGGKYYGNGVHCTKSGCSVNWGEAASAGIHRLANGGNGFW

SEQ ID NO: 195 - DNA, 186 bp, Leuconostoc mesenteroides
  source 1..186: mol_type = genomic DNA
atgacgaatatgaagtctgtggaagcatatcagcaattagataaccagaatctcaagaaagttgttggtggaaagtattatgggaatggtgttcactgtacaaaaagtggatgctctgttaactggggagaagctgcctcagctggcatacatcgtttggccaatggtggaaatggattttggtaa

SEQ ID NO: 196 - protein, 68 aa, Clavibacter michiganensis
  source 1..68: note = subsp. michiganensis
MNDILETETPVMVSPRWDMLLDAGEDTSPSVQTQIDAEFRRVVSPYMSSSGWLCTLTIECGTIICACR

SEQ ID NO: 197 - DNA, 207 bp, Clavibacter michiganensis
  source 1..207: mol_type = genomic DNA; note = subsp. michiganensis
atgaacgacatcctcgagacggagacccccgtcatggtcagcccccggtgggacatgctgctcgacgcgggcgaggacaccagcccgtccgtccagacccagatcgacgcggagttccgtcgcgtcgtgagcccgtacatgtccagcagcggctggctctgcacgctcaccatcgaatgtggcaccatcatctgcgcgtgtcgctga

SEQ ID NO: 198 - protein, 69 aa, Escherichia coli
MELKASEFGVVLSVDALKLSRQSPLGVGIGGGGGGGGGGSCGGQGGGCGGCSNGCSGGNGGSGGSGSHI

SEQ ID NO: 199 - DNA, 207 bp, Escherichia coli
  source 1..207: mol_type = genomic DNA
atggaattaaaagcgagtgaatttggtgtagttttgtccgttgatgctcttaaattatcacgccagtctccattaggtgttggcattggtggtggtggcggcggcggcggcggcggtagctgcggtggtcaaggtggcggttgtggtggttgcagcaacggttgtagtggtggaaacggtggcagcggcggaagtggttcacatatc

SEQ ID NO: 200 - protein, 7 aa, Escherichia coli
MRTGNAN

SEQ ID NO: 201 - DNA, 24 bp, Escherichia coli
  source 1..24: mol_type = genomic DNA
atgcgtactggtaatgcaaactaa

SEQ ID NO: 202 - protein, 99 aa, Klebsiella pneumoniae
MREISQKDLNLAFGAGETDPNTQLLNDLGNNMAWGAALGAPGGLGSAALGAAGGALQTVGQGLIDHGPVNVPIPVLIGPSWNGSGSGYNSATSSSGSGS

SEQ ID NO: 203 - DNA, 300 bp, Klebsiella pneumoniae
  source 1..300: mol_type = genomic DNA
atgagagaaattagtcaaaaggacttaaatcttgcttttggtgcaggagagaccgatccaaatactcaacttctaaacgaccttggaaataatatggcatggggtgctgctcttggcgctcctggcggattaggatcagcagctttgggggccgcgggaggtgcattacaaactgtagggcaaggattaattgaccatggtcctgtaaatgtccccatccctgtactcatcgggccaagctggaatggtagcggtagtggttataacagcgcaacatccagttccggtagtggtagttaa

SEQ ID NO: 204 - protein, 75 aa, Escherichia coli
MREITESQLRYISGAGGAPATSANAAGAAAIVGALAGIPGGPLGVVVGAVSAGLTTAIGSTVGSGSASSSAGGGS

SEQ ID NO: 205 - DNA, 228 bp, Escherichia coli
  source 1..228: mol_type = genomic DNA
atgcgagaaataacagaatcacagttaagatatatttccggggcgggaggtgcgccagcgacttcagctaatgccgcaggtgctgcagctattgttggagctctcgccggaatacctggtggtccacttggggttgtagttggagccgtatctgccggtttgacaacagcaattggctcgaccgtgggaagtggtagtgccagttcttctgctggtggcggtagctaa

SEQ ID NO: 206 - protein, 58 aa, Escherichia coli
MIKHFHFNKLSSGKKNNVPSPAKGVIQIKKSASQLTKGGAGHVPEYFVGIGTPISFYG

SEQ ID NO: 207 - DNA, 177 bp, Escherichia coli
  source 1..177: mol_type = genomic DNA
atgattaagcattttcattttaataaactgtcttctggtaaaaaaaataatgttccatctcctgcaaagggggttatacaaataaaaaaatcagcatcgcaactcacaaaaggtggtgcaggacatgtgcctgagtattttgtggggattggtacacctatatctttctatggctga

SEQ ID NO: 208 - protein, 90 aa, Escherichia coli
MYMRELDREELNCVGGAGDPLADPNSQIVRQIMSNAAWGPPLVPERFRGMAVGAAGGVTQTVLQGAAAHMPVNVPIPKVPMGPSWNGSKG

SEQ ID NO: 209 - DNA, 273 bp, Escherichia coli
  source 1..273: mol_type = genomic DNA
atgtatatgagagagttagatagagaggaattaaattgcgttggtggggctggagatccgcttgcagatcctaattcccaaattgtaagacagataatgtctaatgcggcatggggcccgcctttggtgccagagcggtttaggggaatggctgttggagccgcaggtggggttacgcagacagttcttcaaggagcagcagctcatatgccggttaatgtccctatacctaaagttccgatgggaccctcatggaacggaagtaaaggataa

SEQ ID NO: 210 - protein, 50 aa, Enterococcus mundtii
MSQVVGGKYYGNGVSCNKKGCSVDWGKAIGIIGNNSAANLATGGAAGWKS

SEQ ID NO: 211 - DNA, 153 bp, Enterococcus mundtii
  source 1..153: mol_type = genomic DNA
atgtcacaggtagtaggtggaaaatactacggtaatggagtctcatgtaataaaaaagggtgcagtgttgattggggaaaagcgattggcattattggaaataattctgctgcgaatttagctactggtggagcagctggttggaaaagttaa

SEQ ID NO: 212 - protein, 58 aa, Enterococcus mundtii
MKKLTSKEMAQVVGGKYYGNGLSCNKKGCSVDWGKAIGIIGNNSAANLATGGAAGWKS

SEQ ID NO: 213 - DNA, 177 bp, Enterococcus mundtii
  source 1..177: mol_type = genomic DNA
ttgaagaaattaacatcaaaagaaatggcacaagtagtaggtgggaaatactacggtaatggattatcatgtaataaaaaagggtgcagtgttgattggggaaaagctattggcattattggaaataattctgctgcgaatttagctactggtggagcagctggttggaaaagttaa

SEQ ID NO: 214 - protein, 63 aa, Streptococcus mutans
MSNTQLLEVLGTETFDVQEDLFAFDTTDTTIVASNDDPDTRFKSWSLCTPGCARTGSFNSYCC

SEQ ID NO: 215 - DNA, 192 bp, Streptococcus mutans
  source 1..192: mol_type = genomic DNA
atgtcaaacacacaattattagaagtccttggtactgaaacttttgatgttcaagaagatctctttgcttttgatacaacagatactactattgtggcaagcaacgacgatccagatactcgtttcaaaagttggagcctttgtacgcctggttgtgcaaggacaggtagtttcaatagttactgttgctga

SEQ ID NO: 216 - protein, 53 aa, Streptococcus mutans
MNKLNSNAVVSLNEVSDSELDTILGGNRWWQGVVPTVSYECRMNSWQHVFTCC

SEQ ID NO: 217 - DNA, 162 bp, Streptococcus mutans
  source 1..162: mol_type = genomic DNA
atgaacaagttaaacagtaacgcagtagtttctttgaatgaagtttcagattctgaattggatactattttgggtggtaatcgttggtggcaaggtgttgtgccaacggtctcatatgagtgtcgcatgaattcatggcaacatgttttcacttgctgttaa

SEQ ID NO: 218 - protein, 57 aa, Lactococcus lactis
  source 1..57: note = subsp. lactis
MSTKDFNLDLVSVSKKDSGASPRITSISLCTPGCKTGALMGCNMKTATCHCSIHVSK

SEQ ID NO: 219 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataa

SEQ ID NO: 220 - protein, 57 aa, Lactococcus lactis
MSTKDFNLDLVSVSKKDSGASPRITSISLCTPGCKTGALMGCNMKTATCNCSVHVSK

SEQ ID NO: 221 - DNA, 171 bp, Lactococcus lactis
  source 1..171: mol_type = genomic DNA
atgagtacaaaagatttcaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagcgttcacgtaagcaaa

SEQ ID NO: 222 - protein, 57 aa, Lactococcus lactis
MSTKDFNLDLVSVSKTDSGASTRITSISLCTPGCKTGVLMGCNLKTATCNCSVHVSK

SEQ ID NO: 223 - DNA, 174 bp, Lactococcus lactis
  source 1..174: mol_type = genomic DNA
atgagtacaaaagatttcaacttagatttggtatctgtttcaaaaacagattctggcgcttcaacacgtattaccagcatttcgctttgtacaccaggttgtaaaacaggtgttctgatgggatgtaacctgaaaacagcaacttgtaattgtagcgttcacgtaagcaaataa

SEQ ID NO: 224 - protein, 55 aa, Streptococcus uberis
MNNEDFNLDLIKISKENNSGASPRITSKSLCTPGCKTGILMTCPLKTATCGCHFG

SEQ ID NO: 225 - DNA, 168 bp, Streptococcus uberis
  source 1..168: mol_type = genomic DNA
atgaacaatgaagattttaatttggatctcatcaaaatctcaaaggaaaacaactcaggagcttcacctcgaataactagtaaatcattatgtactcctggatgtaagacgggtattttgatgacttgtccactaaaaactgcaacctgtggttgtcattttggataa

SEQ ID NO: 226 - protein, 57 aa, Lactococcus lactis
  source 1..57: note = subsp. lactis
MSTKDFNLDLVSVSKKDSGASPRITSISLCTPGCKTGALMGCNMKTATCNCSIHVSK

SEQ ID NO: 227 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagtattcacgtaagcaaataa

SEQ ID NO: 228 - protein, 57 aa, Staphylococcus warneri
MENSKVMKDIEVANLLEEVQEDELNEVLGAKKKSGVIPTVSHDCHMNSFQFVFTCCS

SEQ ID NO: 229 - DNA, 174 bp, Staphylococcus warneri
  source 1..174: mol_type = genomic DNA
atggaaaattctaaagttatgaaggacattgaagtagcaaatttattagaagaggttcaagaagatgaattgaatgaagtcttaggagctaagaaaaagtcaggagtaatcccaactgtgtcacacgattgccatatgaattctttccaatttgtatttacttgttgttcataa

SEQ ID NO: 230 - protein, 58 aa, Paenibacillus polymyxa
MAENLFDLDIQVNKSQGSVEPQVLSIVACSSGCGSGKTAASCVETCGNRCFTNVGSLC

SEQ ID NO: 231 - DNA, 177 bp, Paenibacillus polymyxa
  source 1..177: mol_type = genomic DNA
atggctgaaaacttatttgatctggacattcaagtaaacaaatctcaaggttctgtagagcctcaggttctgagcattgttgcatgttctagcggatgtggtagcggtaaaacagctgccagttgtgttgaaacttgtggcaaccggtgctttactaacgttggttcactctgctaa

SEQ ID NO: 232 - protein, 62 aa, Pediococcus acidilactici
MKKIEKLTEKEMANIIGGKYYGNGVTCGKHSCSVDWGKATTCIINNGAMAWATGGHQGNHKC

SEQ ID NO: 233 - DNA, 189 bp, Pediococcus acidilactici
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacttgcataatcaataatggagctatggcatgggctactggtggacatcaaggtaatcataaatgctag

SEQ ID NO: 234 - protein, 60 aa, Pediococcus pentosaceus
MTEIKVLNDKELKNVVGGKYYGNGVHCGKKTCYVDWGQATASIGKIIVNGWTQHGPWAHR

SEQ ID NO: 235 - DNA, 183 bp, Pediococcus pentosaceus
  source 1..183: mol_type = genomic DNA
atgactgaaattaaagtactaaacgataaggaactaaaaaatgtcgtaggaggaaagtattacggtaacggagtgcattgtggtaaaaagacttgctatgtggactggggacaagctacagctagcattggaaaaattatagtgaacggatggacacaacacgggccttgggcacatagataa

SEQ ID NO: 236 - protein, 60 aa, Staphylococcus epidermidis
MKNNKNLFDLEIKKETSQNTDELEPQTAGPAIRASVKQCQKTLKATRLFTVSCKGKNGCK

SEQ ID NO: 237 - DNA, 183 bp, Staphylococcus epidermidis
  source 1..183: mol_type = genomic DNA
atgaaaaataacaaaaatttatttgatttagaaattaaaaaagaaacaagtcaaaacactgatgaacttgaacctcaaactgctggaccagcgattagagcttctgtgaaacaatgtcagaaaactttgaaagctacgcgtttatttacagtgtcttgcaaaggaaaaaacggatgtaaatag

SEQ ID NO: 238 - protein, 62 aa, Carnobacterium maltaromaticum
MKTVKELSVKEMQLTTGGKYYGNGVSCNKNGCTVDWSKAIGIIGNNAAANLTTGGAAGWNKG

SEQ ID NO: 239 - DNA, 189 bp, Carnobacterium maltaromaticum
  source 1..189: mol_type = genomic DNA
atgaaaactgttaaagaacttagcgttaaagaaatgcaactaactacaggaggtaagtattacggaaatggcgtttcctgtaataaaaatggttgtactgtagattggagcaaagctattgggattataggaaacaatgcagcagcaaatttgactacaggtggagccgctggttggaacaaaggataa

SEQ ID NO: 240 - protein, 69 aa, Lactobacillus plantarum
  VARIANT 40: note = Xaa = any amino acid
MYKELTVDELALIDGGKKKKKKVACTWGNAATAAASGAVXGILGGPTGALAGAIWGVSQCASNNLHGMH

SEQ ID NO: 241 - DNA, 210 bp, Lactobacillus plantarum
  source 1..210: mol_type = genomic DNA
  variation 119: note = n = a, c, t or g
atgtataaagaattaacagttgatgaattagcattgattgatggaggaaaaaagaagaagaaaaaagtagcttgtacttggggaaatgcagcaacagccgctgcttctggtgcagttangggtattcttggtgggcctactggtgcactggctggagctatctggggcgtttcacaatgcgcgtctaacaacttacacggcatgcactaa

SEQ ID NO: 242 - protein, 56 aa, Lactobacillus plantarum
MMKKIEKLTEKEMANIIGGKYYGNGVTCGKHSCSVNWGQAFSCSVSHLANFGHGKC

SEQ ID NO: 243 - DNA, 171 bp, Lactobacillus plantarum
  source 1..171: mol_type = genomic DNA
atgatgaaaaaaattgaaaaattaactgaaaaagaaatggccaatatcattggtggtaaatactatggtaatggggttacttgtggtaaacattcctgctctgttaactggggccaagcattttcttgtagtgtgtcacatttagctaacttcggtcatggaaagtgctaa

SEQ ID NO: 244 - protein, 64 aa, Lactobacillus plantarum
MSKLVKTLTVDEISKIQTNGGKPAWCWYTLAMCGAGYDSGTCDYMYSHCFGVKHSSGGGGSYHC

SEQ ID NO: 245 - DNA, 195 bp, Lactobacillus plantarum
  source 1..195: mol_type = genomic DNA
atgagtaaactagttaaaacattaactgtcgatgaaatctctaagattcaaaccaatggtggaaaacctgcatggtgttggtacacattggcaatgtgcggtgctggttatgattcaggcacttgtgattatatgtattcacactgctttggtgtaaaacactctagcggtggtggcggtagctaccattgttag

SEQ ID NO: 246 - protein, 56 aa, Lactobacillus plantarum
MLQFEKLQYSRLPQKKLAKISGGFNRGGYNFGKSVRHVVDAIGSVAGIRGILKSIR

SEQ ID NO: 247 - DNA, 171 bp, Lactobacillus plantarum
  source 1..171: mol_type = genomic DNA
atgctacagtttgagaaattacaatattccaggttgccgcaaaaaaagcttgccaaaatatctggtggttttaatcggggcggttataactttggtaaaagtgttcgacatgttgttgatgcaattggttcagttgcaggcattcgtggtattttgaaaagtattcgttaa

SEQ ID NO: 248 - protein, 52 aa, Lactobacillus plantarum
MKKFLVLRDRELNAISGGVFHAYSARGVRNNYKSAVGPADWVISAVRGFIHG

SEQ ID NO: 249 - DNA, 159 bp, Lactobacillus plantarum
  source 1..159: mol_type = genomic DNA
atgaaaaaatttctagttttgcgtgaccgtgaattaaatgctatttcaggtggcgttttccatgcctatagcgcgcgtggcgttcggaataattataaaagtgctgttgggcctgccgattgggtcattagcgctgtccgaggattcatccacggatag

SEQ ID NO: 250 - protein, 55 aa, Lactobacillus plantarum
MTVNKMIKDLDVVDAFAPISNNKLNGVVGGGAWKNFWSSLRKGFYDGEAGRAIRR

SEQ ID NO: 251 - DNA, 168 bp, Lactobacillus plantarum
  source 1..168: mol_type = genomic DNA
atgactgtgaacaaaatgattaaggatttggatgtagtagatgcatttgcacctatttctaataataagttgaacggggttgttgggggaggcgcttggaaaaatttctggtctagtttaagaaaaggattttatgatggcgaagctggcagagcaatccgtcgttaa

SEQ ID NO: 252 - protein, 57 aa, Lactobacillus plantarum
MKIKLTVLNEFEELTADAEKNISGGRRSRKNGIGYAIGYAFGAVERAVLGGSRDYNK

SEQ ID NO: 253 - DNA, 174 bp, Lactobacillus plantarum
  source 1..174: mol_type = genomic DNA
atgaaaattaaattaactgttttaaatgaatttgaagaattaactgctgacgctgaaaagaatatttctggtggccgtcggagtcgtaaaaatggaattggatacgctattggttatgcgtttggcgcggttgaacgggccgtgcttggtggttcaagggattataataagtga

SEQ ID NO: 254 - protein, 47 aa, Lactobacillus plantarum
MDKFEKISTSNLEKISGGDLTTKLWSSWGYYLGKKARWNLKHPYVQF

SEQ ID NO: 255 - DNA, 141 bp, Lactobacillus plantarum
  source 1..141: mol_type = genomic DNA
atggataaatttgaaaaaattagtacatctaacctagaaaagatctctggcggtgatttaacaaccaagttatggagctcttggggatattatcttggcaagaaagcacgttggaatttaaagcacccatatgttcaattt

SEQ ID NO: 256 - protein, 55 aa, Lactobacillus plantarum
MNNLNKFSTLGKSSLSQIEGGSVPTSVYTLGIKILWSAYKHRKTIEKSFNKGFYH

SEQ ID NO: 257 - DNA, 168 bp, Lactobacillus plantarum
  source 1..168: mol_type = genomic DNA
atgaataacttgaataaattttctactctaggcaagagtagcttgtctcaaattgagggcggatcagtcccaacttcagtatatacgcttggaattaaaattctatggtctgcgtataagcatcgcaaaacgattgaaaaaagttttaataaaggcttttatcattaa

SEQ ID NO: 258 - protein, 55 aa, Lactobacillus plantarum
MNNALSFEQQFTDFSTLSDSELESVEGGRNKLAYNMGHYAGKATIFGLAAWALLA

SEQ ID NO: 259 - DNA, 168 bp, Lactobacillus plantarum
  source 1..168: mol_type = genomic DNA
atgaataacgcattaagttttgaacaacaatttacagacttcagcaccttatcggactctgaattagaatccgttgagggtggccgaaataagcttgcatataatatggggcattacgctggtaaggcaaccatttttggacttgcagcatgggcactccttgcatga

SEQ ID NO: 260 - protein, 47 aa, Lactobacillus plantarum
MDKIIKFQGISDDQLNAVIGGKKKKQSWYAAAGDAIVSFGEGFLNAW

SEQ ID NO: 261 - DNA, 144 bp, Lactobacillus plantarum
  source 1..144: mol_type = genomic DNA
atggataagattattaagtttcaagggatttctgatgatcaattaaatgctgttatcggtgggaaaaagaaaaaacaatcttggtacgcagcagctggtgatgcaatcgttagttttggtgaaggatttttaaatgcttggtaa

SEQ ID NO: 262 - protein, 59 aa, Lactobacillus plantarum
MKISKIEAQARKDFFKKIDTNSNLLNVNGAKCKWWNISCDLGNNGHVCTLSHECQVSCN

SEQ ID NO: 263 - DNA, 180 bp, Lactobacillus plantarum
  source 1..180: mol_type = genomic DNA
atgaaaatttctaagattgaagctcaggctcgtaaagatttttttaaaaaaatcgatactaactcgaacttattaaatgtaaatggtgccaaatgcaagtggtggaatatttcgtgtgatttaggaaataatggccatgtttgtaccttgtcacatgaatgccaagtatcttgtaactaa

SEQ ID NO: 264 - protein, 67 aa, Lactobacillus plantarum
MTKTSRRKNAIANYLEPVDEKSINESFGAGDPEARSGIPCTIGAAVAASIAVCPTTKCSKRCGKRKK

SEQ ID NO: 265 - DNA, 204 bp, Lactobacillus plantarum
  source 1..204: mol_type = genomic DNA
atgactaaaactagtcgtcgtaagaatgctattgctaattatttagaaccagtcgacgaaaaaagtattaatgaatcttttggggctggggatccggaagcaagatccggaattccatgtacaatcggcgcagctgtcgcagcatcaattgcagtttgtccaactactaagtgtagtaaacgttgtggcaagcgtaagaaataa

SEQ ID NO: 266 - protein, 48 aa, Lactobacillus plantarum
MKIQIKGMKQLSNKEMQKIVGGKSSAYSLQMGATAIKQVKKLFKKWGW

SEQ ID NO: 267 - DNA, 147 bp, Lactobacillus plantarum
  source 1..147: mol_type = genomic DNA
atgaaaattcaaattaaaggtatgaagcaacttagtaataaggaaatgcaaaaaatagtaggtggaaagagtagtgcgtattctttgcagatgggggcaactgcaattaaacaggtaaagaaactgtttaaaaaatggggatggtaa

SEQ ID NO: 268 - protein, 207 aa, Propionibacterium jensenii
MNKTHKMATLVIAAILAAGMTAPTAYADSPGNTRITASEQSVLTQILGHKPTQTEYNRYVETYGSVPTEADINAYIEASESEGSSSQTAAHDDSTSPGTSTEIYTQAAPARFSMFFLSGTWITRSGVVSLSLKPRKGGIGNEGDERTWKTVYDKFHNAGQWTRYKNNGVDASMKKQYMCHFKYGMVKTPWNLEPHKKAADVSPVKCN

SEQ ID NO: 269 - DNA, 624 bp, Propionibacterium jensenii
  source 1..624: mol_type = genomic DNA
atgaacaaaacacacaaaatggcgacgctggtaattgccgcgatcttggccgccggaatgaccgcaccaactgcctatgcagattctcctggaaacaccagaattacagccagcgagcaaagcgtccttacccagatactcggccacaaacctacacaaactgaatataaccgatacgttgagacttacggaagcgtaccgaccgaagcagacatcaacgcatatatagaagcgtctgaatctgagggatcatcaagtcaaacggctgctcacgatgactcgacatcacccggcacgagtaccgaaatctacacgcaggcagcccctgccaggttctcaatgtttttcctgtccggaacttggatcactaggagtggtgtagtatcgctctccttgaagccaaggaagggtggtattggcaacgagggggacgagcgtacctggaagactgtatacgacaaattccataacgctgggcaatggacacgatacaagaacaacggcgtagacgccagcatgaaaaagcagtacatgtgccacttcaagtacgggatggtgaagacgccatggaatctggagccccacaagaaggctgcagacgtcagtccagtcaagtgcaactag

SEQ ID NO: 270 - protein, 96 aa, Propionibacterium thoenii
MKKTLLRSGTIALATAAAFGASLAAAPSAMAVPGGCTYTRSNRDVIGTCKTGSGQFRIRLDCNNAPDKTSVWAKPKVMVSVHCLVGQPRSISFETK

SEQ ID NO: 271 - DNA, 291 bp, Propionibacterium thoenii
  source 1..291: mol_type = genomic DNA
atgaagaagaccctcctgcgaagtggaacgatcgcactggcgaccgcggctgcatttggcgcatcattggcagccgccccatctgccatggccgttcctggtggttgcacgtacacaagaagcaatcgcgacgtcatcggtacctgcaagactggaagcggccagttccgaatccgacttgactgcaacaacgctccagacaaaacttcagtctgggccaagcccaaggtaatggtgtcggttcactgtcttgttggtcaaccgaggtccatctcgttcgagaccaagtga

SEQ ID NO: 272 - protein, 255 aa, Propionibacterium freudenreichii
  source 1..255: note = subsp. freudenreii
MNTKAVNLKSENTTKLVSYLTENQLDEFIRRIRIDGALVEEVSQNAKQALDNTGLNGWINTDCDEGLLSDFISKIASARWIPLAESIRPAVTDRDKYRVSCWFYQGMNIAIYANIGGVANIIGYTEAAVATLLGAVVAVAPVVPGTPTPPKDKSSQYKEVPLAVRLSETYHEEGVRGLFDELNYSESRMISTLRRASTDGVLINSWNDGQDTILLKKYNFQDLQLTVRSRIVGNQTIIEECKITDGRKTLSDETV

SEQ ID NO: 273 - DNA, 768 bp, Propionibacterium freudenreichii
  source 1..768: mol_type = genomic DNA; note = subsp. freudenreii
atgaataccaaagctgtaaatctgaagtcagaaaacacgactaagttggtgagctaccttacggaaaatcaattggatgagtttattagaaggattcgcattgatggcgctcttgtggaagaggtcagtcaaaatgctaagcaggccttagataatactgggctcaatggctggataaatactgattgcgatgaaggccttctctctgatttcatttcaaagatagcaagtgctagatggattccattagctgagtcaattcgacctgcggtgactgacagggataagtatcgagtaagttgctggttctaccaggggatgaatatagcaatttacgcaaatatcggtggcgtggccaatattatcggctatacggaggccgcagtcgcaacactccttggtgcagttgtggcggtagctcctgtggtccctggaactccaacccctccaaaggacaagagttcgcaatataaggaggttccccttgccgttcgtctttccgaaacataccacgaagagggagtacgaggtctattcgacgagctgaactactccgagagccgtatgatctctactctaaggcgagcatcaaccgatggagtcctaattaattcttggaacgatgggcaggatacaattctgcttaagaagtacaatttccaagacttgcaactgactgtcaggagccgcattgttgggaatcaaacaataattgaagaatgcaaaatcactgatggtagaaaaactctttcagacgagactgtgtag

SEQ ID NO: 274 - protein, 618 aa, Pseudomonas aeruginosa
MARPIADLIHFNSTTVTASGDVYYGPGGGTGIGPIARPIEHGLDSSTENGWQEFESYADVGVDPRRYVPLQVKEKRREIELQFRDAEKKLEASVQAELDKADAALGPAKNLAPLDVINRSLTIVGNALQQKNQKLLLNQKKITSLGAKNFLTRTAEEIGEQAVREGNINGPEAYMRFLDREMEGLTAAYNVKLFTEAISSLQIRMNTLTAAKASIEAAAANKAREQAAAEAKRKAEEQARQQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRVADGGGVYNMGNLVAVTPKRHIEIHKGGK

SEQ ID NO: 275 - DNA, 1857 bp, Pseudomonas aeruginosa
  source 1..1857: mol_type = genomic DNA
atggcacgacccattgctgaccttatccacttcaactctacaactgtcacggcaagcggagacgtttattacggccctgggggaggtaccggcattggccccattgccagacctatagagcacggcttggattcgtccactgaaaatggctggcaagagtttgaaagttatgctgatgtgggcgttgaccccagacgctatgttcctcttcaggttaaagaaaaacgcagggagatcgagcttcagttccgagatgccgagaaaaaacttgaggcgtcggtacaagccgagctggataaggctgatgccgctcttggtccggcaaagaatcttgcaccattggacgtcatcaaccgcagtctgaccatcgttggaaacgccctccagcaaaagaatcaaaaactactgctgaatcagaagaagattaccagcctgggtgcaaagaatttccttacccgtacggcggaagagatcggtgaacaagcggtgcgagaaggcaatattaacgggcctgaagcctatatgcgcttcctcgacagggaaatggaaggtctcacggcagcttataacgtaaaactcttcaccgaagcgatcagtagtctccagatccgcatgaatacgttgaccgccgccaaagcaagtattgaggcggccgcagcaaacaaggcgcgtgaacaagcagcggctgaggccaaacgcaaagccgaagagcaggcccgccagcaagcggcgataagagctgccaatacctatgccatgccggccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgagtagcagatggaggcggcgtttacaatatggggaaccttgttgcagtaacgccaaaacgtcatatagaaatccacaagggagggaagtga

SEQ ID NO: 276 - protein, 689 aa, Pseudomonas aeruginosa
MAVNDYEPGSMVITHVQGGGRDIIQYIPARSSYGTPPFVPPGPSPYVGTGMQEYRKLRSTLDKSHSELKKNLKNETLKEVDELKSEAGLPGKAVSANDIRDEKSIVDALMDAKAKSLKAIEDRPANLYTASDFPQKSESMYQSQLLASRKFYGEFLDRHMSELAKAYSADIYKAQIAILKQTSQELENKARSLEAEAQRAAAEVEADYKARKANVEKKVQSELDQAGNALPQLTNPTPEQWLERATQLVTQAIANKKKLQTANNALIAKAPNALEKQKATYNADLLVDEIASLQARLDKLNAETARRKEIARQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRIADGGGVYNMGNLVAVTPKRHIEIHKGGK

SEQ ID NO: 277 - DNA, 2070 bp, Pseudomonas aeruginosa
  source 1..2070: mol_type = genomic DNA
atggctgtcaatgattacgaacctggttcgatggttattacacatgtgcagggtggtgggcgtgacataatccagtatattcctgctcgatcaagctacggtactccaccatttgtcccaccaggaccaagtccgtatgtcggtactggaatgcaggagtacaggaagctaagaagtacgcttgataagtcccattcagaactcaagaaaaacctgaaaaatgaaaccctgaaggaggttgatgaactcaagagtgaagcggggttgccaggtaaagcggtcagtgccaatgacatccgcgatgaaaagagtatcgttgatgcactcatggatgccaaagcaaaatcgctaaaggccattgaggatcgcccggccaatctttatacggcttcagactttcctcagaagtcagagtcgatgtaccagagtcagttgctggccagccgaaaattctatggagagttcctggatcgccatatgagtgagctggccaaagcgtacagcgccgatatctataaggcgcaaatcgctatcttgaaacaaacgtctcaagagctggagaataaagcccggtcattggaagcagaagcccagcgagccgctgctgaggtggaggcggactacaaggccaggaaggcaaatgtcgagaaaaaagtgcagtccgagcttgaccaggctgggaatgctttgcctcaactgaccaatccaacgccagagcagtggcttgaacgcgctactcaactggttacgcaggcgatcgccaataagaagaaattgcagactgcaaacaatgccttgattgccaaggcacccaatgcactggagaaacaaaaggcaacctacaacgccgatctcctagtggatgaaatcgccagcctgcaagcacggctggacaagctgaacgccgaaacggcaaggcgcaaggaaatcgctcgtcaagcggcgatcagggctgccaatacttatgccatgccagccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgaatagcagatggaggcggcgtttacaatatggggaaccttgttgcagtaacgccaaaacgtcatatagaaatccacaagggagggaagtga

SEQ ID NO: 278 - protein, 47 aa, Ruminococcus gnavus
MRNDVLTLTNPMEEKELEQILGGGNGVLKTISHECNMNTWQFLFTCC

SEQ ID NO: 279 - DNA, 144 bp, Ruminococcus gnavus
  source 1..144: mol_type = genomic DNA
atgagaaatgacgtattaacattaacaaacccaatggaagagaacgaactggagcagatcttaggtggtggcaatggtgtgttaaaaacgattagccacgaatgcaatatgaacacatggcagttcctgtttacttgttgctaa

SEQ ID NO: 280 - protein, 55 aa, Lactobacillus sakei
MKNAKSLTIQEMKSITGGKYYGNGVSCNSHGCSVNWGQAWTCGVNHLANGGHGVC

SEQ ID NO: 281 - DNA, 168 bp, Lactobacillus sakei
  source 1..168: mol_type = genomic DNA
atgaaaaacgcaaaaagcctaacaattcaagaaatgaaatctattacaggtggtaaatactatggtaatggcgttagctgtaactctcacggctgttcagtaaattgggggcaagcatggacttgtggagtaaaccatctagctaatggcggtcatggagtttgttaa

SEQ ID NO: 282 - protein, 59 aa, Lactobacillus sakei; identical to SEQ ID NO: 68

SEQ ID NO: 283 - DNA, 180 bp, Lactobacillus sakei; identical to SEQ ID NO: 69
  source 1..180: mol_type = genomic DNA

SEQ ID NO: 284 - protein, 61 aa, Lactobacillus sakei
MEKFIELSLKEVTAITGGKYYGNGVHCGKHSCTVDWGTAIGNIGNNAAANWATGGNAGWNK

SEQ ID NO: 285 - DNA, 186 bp, Lactobacillus sakei
  source 1..186: mol_type = genomic DNA
atggaaaagtttattgaattatctttaaaagaagtaacagcaattacaggtggaaaatattatggtaacggtgtacactgtggaaaacattcatgtaccgtagactggggaacagctattggaaatatcggaaataatgcagctgcaaactgggccacaggcggaaacgctggctggaataaataa

SEQ ID NO: 286 - protein, 56 aa, Streptococcus salivarius
MKSTNNQSIAEIAAVNSLQEVSMEELDQIIGAGNGVVLTLTHECNLATWTKKLKCC

SEQ ID NO: 287 - DNA, 171 bp, Streptococcus salivarius
  source 1..171: mol_type = genomic DNA
atgaaatcaacaaataatcaaagtatcgcagaaattgcagcagtaaactcactacaagaagtaagtatggaggaactagaccaaattattggtgccggaaacggagtggttcttactcttactcatgaatgtaacctagcaacttggacaaaaaaactaaaatgttgctaa

SEQ ID NO: 288 - protein, 51 aa, Streptococcus pyogenes
  source 1..51: note = serotype M28
MSFMKNSKDILTNAIEEVSEKELMEVAGGKKGSGWFATITDDCPNSVFVCC

SEQ ID NO: 289 - DNA, 156 bp, Streptococcus pyogenes
  source 1..156: mol_type = genomic DNA; note = serotype M28
atgagttttatgaaaaattcaaaggatattttgactaatgctatcgaagaagtttctgaaaaagaacttatggaagtagctggtggtaaaaaaggttccggttggtttgcaactattactgatgactgtccgaactcagtattcgtttgttgttaa

SEQ ID NO: 290 - protein, 48 aa, Streptococcus salivarius
MKNSKDVLNNAIEEVSEKELMEVAGGKKGPGWIATITDDCPNSIFVCC

SEQ ID NO: 291 - DNA, 147 bp, Streptococcus salivarius
  source 1..147: mol_type = genomic DNA
atgaaaaactcaaaagatgttttgaacaatgctatcgaagaggtttctgaaaaagaacttatggaagtagctggtggtaaaaaaggtccaggttggattgcaactattactgatgactgtccaaactcaatattcgtttgttgttaa

SEQ ID NO: 292 - protein, 48 aa, Streptococcus salivarius
MKNSKDILNNAIEEVSEKELMEVAGGKRGSGWIATITDDCPNSVFVCC

SEQ ID NO: 293 - DNA, 147 bp, Streptococcus salivarius
  source 1..147: mol_type = genomic DNA
atgaaaaactcaaaagatattttgaacaatgctatcgaagaagtttctgaaaaagaacttatggaagtagctggtggtaaaagaggttcaggttggattgcaactattactgatgactgtccaaactcagtattcgtttgttgttaa

SEQ ID NO: 294 - protein, 62 aa, Staphylococcus aureus
MKSSFLEKDIEEQVTWFEEVSEQEFDDDIFGACSTNTFSLSDYWGNKGNWCTATHECMSWCK

SEQ ID NO: 295 - DNA, 189 bp, Staphylococcus aureus
  source 1..189: mol_type = genomic DNA
atgaaaagttcttttttagaaaaagatatagaagaacaagtgacatggttcgaggaagtttcagaacaagaatttgacgatgatatttttggagcttgtagtacaaacactttttctttgagtgactattggggtaataaaggaaattggtgtactgctactcacgaatgtatgtcttggtgtaaataa

SEQ ID NO: 296 - protein, 67 aa, Staphylococcus aureus
MKNELGKFLEENELELGKFSESDMLEITDDEVYAAGTPLALLGGAATGVIGYISNQTCPTTACTRAC

SEQ ID NO: 297 - DNA, 204 bp, Staphylococcus aureus
  source 1..204: mol_type = genomic DNA
atgaaaaatgaattaggtaagtttttagaagaaaacgaattagagttaggtaaattttcagaatcagacatgctagaaattactgatgatgaagtatatgcagctggaacacctttagccttattgggtggagctgccaccggggtgataggttatatttctaaccaaacatgtccaacaactgcttgtacacgcgcttgctag

SEQ ID NO: 298 - protein, 46 aa, Streptococcus pyogenes
MNNTIKDFDLDLKTNKKDTATPYVGSRYLCTPGSCWKLVCFTTTVK

SEQ ID NO: 299 - DNA, 141 bp, Streptococcus pyogenes
  source 1..141: mol_type = genomic DNA
atgaataacacaattaaagactttgatctcgatttgaaaacaaataaaaaagacactgctacaccttatgttggtagccgttacctatgtacccctggttcttgttggaaattagtttgctttacaacaactgttaaataa

SEQ ID NO: 300 - protein, 51 aa, Streptococcus pyogenes
MEKNNEVINSIQEVSLEELDQIIGAGKNGVFKTISHECHLNTWAFLATCCS

SEQ ID NO: 301 - DNA, 156 bp, Streptococcus pyogenes
  source 1..156: mol_type = genomic DNA
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgttcataa

SEQ ID NO: 302 - protein, 51 aa, Streptococcus pyogenes
  source 1..51: note = serotype M49
MTKEHEIINSIQEVSLEELDQIIGAGKNGVFKTISHECHLNTWAFLATCCS

SEQ ID NO: 303 - DNA, 156 bp, Streptococcus pyogenes
  source 1..156: mol_type = genomic DNA; note = serotype M49
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgctcataa

SEQ ID NO: 304 - protein, 56 aa, Bacillus subtilis
MEKLFKEVKLEELENQKGSGLGKAQCAALWLQCASGGTIGCGGGAVACQNYRQFCR

SEQ ID NO: 305 - DNA, 171 bp, Bacillus subtilis
  source 1..171: mol_type = genomic DNA
atggaaaagctatttaaagaagttaaactagaggaactcgaaaaccaaaaaggtagtggattaggaaaagctcagtgtgctgcgttgtggctacaatgtgctagtggcggtacaattggttgtggtggcggagctgttgcttgtcaaaactatcgtcaattctgcagataa

SEQ ID NO: 306 - protein, 56 aa, Bacillus subtilis
MSKFDDFDLDVVKVSKQDSKITPQWKSESLCTPGCVTGALQTCFLQTLTCNCKISK

SEQ ID NO: 307 - DNA, 171 bp, Bacillus subtilis
  source 1..171: mol_type = genomic DNA
atgtcaaagttcgatgatttcgatttggatgttgtgaaagtctctaaacaagactcaaaaatcactccgcaatggaaaagtgaatcactttgtacaccaggatgtgtaactggtgcattgcaaacttgcttccttcaaacactaacttgtaactgcaaaatctctaaataa

SEQ ID NO: 308 - protein, 50 aa, Bacillus subtilis
MKLPVQQVYSVYGGKDLPKGHSHSTMPFLSKLQFLTKIYLLDIHTQPFFI

SEQ ID NO: 309 - DNA, 153 bp, Bacillus subtilis
  source 1..153: mol_type = genomic DNA
ttgaaattgccggtgcaacaggtctattcggtctatgggggtaaggatctcccaaaagggcatagtcattctactatgccctttttaagtaaattacaatttttaactaaaatctacctcttggatatacatacacaaccgtttttcatttga

SEQ ID NO: 310 - protein, 43 aa, Bacillus subtilis
MKKAVIVENKGCATCSIGAACLVDGPIPDFEIAGATGLFGLWG

SEQ ID NO: 311 - DNA, 132 bp, Bacillus subtilis
  source 1..132: mol_type = genomic DNA
atgaaaaaagctgtcattgtagaaaacaaaggttgtgcaacatgctcgatcggagccgcttgtctagtggacggtcctatccctgattttgaaattgccggtgcaacaggtctattcggtctatgggggtaa

SEQ ID NO: 312 - protein, 58 aa, Streptococcus thermophilus; identical to SEQ ID NO: 24

SEQ ID NO: 313 - DNA, 177 bp, Streptococcus thermophilus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcctgtaaaaattgtgcttaa

SEQ ID NO: 314 - protein, 64 aa, Streptococcus thermophilus
MKQYNGFEVLHELDLANVTGGQINWGSVVGHCIGGAIIGGAFSGGAAAGVGCLVGSGKAIINGL

SEQ ID NO: 315 - DNA, 195 bp, Streptococcus thermophilus
  source 1..195: mol_type = genomic DNA
atgaagcagtataatggttttgaggttctacatgaacttgacttagcaaatgtaactggcggtcaaattaattggggatcagttgtaggacactgtataggtggagctattatcggaggtgcattttcaggaggtgcagcggctggagtaggatgccttgttgggagcggaaaggcaatcataaatggattataa

SEQ ID NO: 316 - protein, 85 aa, Streptococcus thermophilus
MNTITICKFDVLDAELLSTVEGGYSGKDCLKDMGGYALAGAGSGALWGAPAGGVGALPGAFVGAHVGAIAGGFACMGGMIGNKFN

SEQ ID NO: 317 - DNA, 258 bp, Streptococcus thermophilus
  source 1..258: mol_type = genomic DNA
atgaatacaataactatttgtaaatttgatgttttagatgctgaacttctttcgacagttgagggtggatactctggtaaggattgtttaaaagacatgggaggatatgcattggcaggagctggaagtggagctctgtggggagctccagcaggaggtgttggagcacttccaggtgcatttgtcggagctcatgttggggcaattgcaggaggctttgcatgtatgggtggaatgattggtaataagtttaactaa

SEQ ID NO: 318 - protein, 52 aa, Bacillus cereus
MSEIKKALNTLEIEDFDAIEMVDVDAMPENEALEIMGASCTTCVCTCSCCTT

SEQ ID NO: 319 - DNA, 159 bp, Bacillus cereus
  source 1..159: mol_type = genomic DNA
atgagtgaaattaaaaaagcattaaatacgcttgaaattgaagattttgatgcaattgaaatggttgatgttgatgctatgccagaaaacgaagcgcttgaaattatgggagcgtcatgtacgacatgcgtatgtacatgcagttgttgtacaacttga

SEQ ID NO: 320 - protein, 47 aa, Bacillus cereus
MEVMNNALITKVDEEIGGNAACVIGCIGSCVISEGIGSLVGTAFTLG

SEQ ID NO: 321 - DNA, 144 bp, Bacillus cereus
  source 1..144: mol_type = genomic DNA
atggaagttatgaacaatgctttaattacaaaagtagatgaggagattggaggaaacgctgcttgtgtaattggttgtattggcagttgcgtaattagtgaaggaattggttcacttgtaggaacagcatttactttaggttaa

SEQ ID NO: 322 - protein, 49 aa, Bacillus cereus
MEVLNKQNVNIIPESEEVGGWVACVGACGTVCLASGGVGTEFAAASYFL

SEQ ID NO: 323 - DNA, 150 bp, Bacillus cereus
  source 1..150: mol_type = genomic DNA
atggaagttttaaacaaacaaaatgtaaatattattccagaatctgaagaagtaggtggatgggtagcatgtgttggagcatgtggtacagtatgtcttgctagtggtggtgttggaacagagtttgcagctgcatcttatttcctataa

SEQ ID NO: 324 - protein, 40 aa, Bacillus thuringiensis
METPVVQPRDWTCWSCLVCAACSVELLNLVTAATGASTAS

SEQ ID NO: 325 - DNA, 123 bp, Bacillus thuringiensis
  source 1..123: mol_type = genomic DNA
atggaaacaccagtagtacaaccaagggattggacttgttggagttgcttagtatgtgcagcatgttctgtggaattattaaatttagttactgcggcaacaggggctagtactgcaagctaa

SEQ ID NO: 326 - protein, 42 aa, Rhizobium leguminosarum
  source 1..42: note = bv. trifolii
MDNKVAKNVEVKKGSIKATFKAAVLKSKTKVDIGGSRQGCVA

SEQ ID NO: 327 - DNA, 129 bp, Rhizobium leguminosarum
  source 1..129: mol_type = genomic DNA; note = bv. trifolii
atggataacaaggttgcgaagaatgtcgaagtgaagaagggctccatcaaggcgaccttcaaggctgctgttctgaagtcgaagacgaaggtcgacatcggaggtagccgtcagggctgcgtcgcttaa

SEQ ID NO: 328 - protein, 70 aa, Streptococcus uberis
MNTIEKFENIKLFSLKKIIGGKTVNYGNGLYCNQKKCWVNWSETATTIVNNSIMNGLTGGNAGWHSGGRA

SEQ ID NO: 329 - DNA, 213 bp, Streptococcus uberis
  source 1..213: mol_type = genomic DNA
atgaatacaattgaaaaatttgaaaatattaaacttttttcactaaagaaaattatcggtggcaaaactgtaaattatggtaatggcctttattgtaaccaaaaaaaatgctgggtaaactggtcagaaactgctacaacaatagtaaataattccatcatgaacgggctcacaggtggtaatgcgggttggcactcaggcgggagagcataa

SEQ ID NO: 330 - protein, 76 aa, Streptococcus uberis
MDILLELAGYTGIASGTAKKVVDAIDKGAAAFVIISIISTVISAGALGAVSASADFIILTVKNYISRNLKAQAVIW

SEQ ID NO: 331 - DNA, 231 bp, Streptococcus uberis
  source 1..231: mol_type = genomic DNA
atggacattttattagaactcgcaggatatactgggatagcctcaggtactgcaaaaaaagttgttgatgccattgataaaggagctgcagcctttgttattatttcaattatctcaacagtaattagtgcgggagcattgggagcagtttcagcctcagctgattttattattttaactgtaaaaaattacattagtagaaatttaaaagcacaagctgtcatttggtaa

SEQ ID NO: 332 - protein, 64 aa, Clostridium perfringens
MDSELFKLMATQGAFAILFSYLLFYVLKENSKREDKYQNIIEELTELLPKIKEDVEDIKEKLNK

SEQ ID NO: 333 - DNA, 195 bp, Clostridium perfringens
  source 1..195: mol_type = genomic DNA
atggatagtgaattatttaagttaatggcaacacaaggagcctttgcaatattattttcgtatttattgttttatgttttaaaagagaatagtaaaagagaagataagtatcaaaatataatagaggagcttacagaattattgccaaaaataaaagaagatgtagaagatataaaagaaaaacttaataaatag

SEQ ID NO: 334 - protein, 47 aa, Micrococcus varians
MTNAFQALDEVTDAELDAILGGGSGVIPTISHECHMNSFQFVFTCCS

SEQ ID NO: 335 - DNA, 144 bp, Micrococcus varians
  source 1..144: mol_type = genomic DNA
atgacgaacgcatttcaggcactggacgaagtcacggacgccgagctcgacgccatccttggcgggggcagtggtgttattcccacgatcagccacgagtgccacatgaactccttccagttcgtgttcacctgctgctcctga

SEQ ID NO: 336 - protein, 285 aa, Streptococcus equi
  source 1..285: note = subsp. zooepidemicus
MKRIFFAFLSLCLFIFGTQTVSAATYTRPLDTGNITTGFNGYPGHVGVDYAVPVGTPVRAVANGTVKFAGNGANHPWMLWMAGNCVLIQHADGMHTGYAHLSKISVSTDSTVKQGQIIGYTGATGQVTGPHLHFEMLPANPNWQNGFSGRIDPTGYIANAPVFNGTTPTEPTTPTTNLKIYKVDDLQKINGIWQVRNNILVPTDFTWVDNGIAADDVIEVTSNGTRTSDQVLQKGGYFVINPNNVKSVGTPMKGSGGLSWAQVNFTTGGNVWLNTTSKDNLLYGK

SEQ ID NO: 337 - DNA, 858 bp, Streptococcus equi
  source 1..858: mol_type = genomic DNA; note = subsp. zooepidemicus
atgaaacgtatattttttgctttcttaagtttatgcttatttatattcggaacacaaacggtatctgcagctacttatactcggccattagatacgggaaatatcactacagggtttaacggataccctggtcatgttggagtcgattatgcagtacccgttggaactccggttagagcagttgcaaatggtacagtcaaatttgcaggtaatggggctaatcacccatggatgctttggatggctggaaactgtgttctaattcaacatgctgacgggatgcatactggatatgcacacttatcaaaaatttcagttagcacagatagtacagttaaacaaggacaaatcataggttatactggtgccaccggccaagttaccggtccacatttgcattttgaaatgttgccagcaaatcctaactggcaaaatggtttttctggaagaatagatccaaccggatacatcgctaatgcccctgtatttaatggaacaacacctacagaacctactactcctacaacaaatttaaaaatctataaagttgatgatttacaaaaaattaatggtatttggcaagtaagaaataacatacttgtaccaactgatttcacatgggttgataatggaattgcagcagatgatgtaattgaagtaactagcaatggaacaagaacctctgaccaagttcttcaaaaaggtggttattttgtcatcaatcctaataatgttaaaagtgttggaactccgatgaaaggtagtggtggtctatcttgggctcaagtaaactttacaacaggtggaaatgtctggttaaatactactagcaaagacaacttactttacggaaaataa

SEQ ID NO: 338 - protein, 45 aa, Myxococcus fulvus
ANCSCSTASDYCPILTFCTTGTACSYTPTGCGTGWVYCACNGNFY

SEQ ID NO: 339 - DNA, 135 bp, synthetic construct
  misc_feature 1..135: note = Polynucleotide encoding the polypeptide of SEQ IDNO: 338
  source 1..135: mol_type = other DNA
gcgaactgcagctgcagcaccgcgagcgattattgcccgattctgaccttttgcaccaccggcaccgcgtgcagctataccccgaccggctgcggcaccggctgggtgtattgcgcgtgcaacggcaacttttat

SEQ ID NO: 340 - protein, 19 aa, Streptomyces griseoluteus
CANSCSYGPLTWSCDGNTK

SEQ ID NO: 341 - DNA, 57 bp, synthetic construct
  misc_feature 1..57: note = Polynucleotide encoding the polypeptide of SEQ IDNO: 340
  source 1..57: mol_type = other DNA
tgcgcgaacagctgcagctatggcccgctgacctggagctgcgatggcaacaccaaa

SEQ ID NO: 342 - protein, 19 aa, Streptoverticillium griseoverticillatum
CKQSCSFGPFTFVCDGNTK

SEQ ID NO: 343 - DNA, 57 bp, synthetic construct
  misc_feature 1..57: note = Polynucleotide encoding the polypeptide of SEQ IDNO: 342
  source 1..57: mol_type = other DNA
tgcaaacagagctgcagctttggcccgtttacctttgtgtgcgatggcaacaccaaa

SEQ ID NO: 344 - protein, 7 aa, Carnobacterium sp.
GSEIQPR

SEQ ID NO: 345 - DNA, 21 bp, synthetic construct
  misc_feature 1..21: note = Polynucleotide encoding the polypeptide of SEQ IDNO: 344
  source 1..21: mol_type = other DNA
ggcagcgaaattcagccgcgc

SEQ ID NO: 346 - protein, 39 aa, Lactococcus lactis
  source 1..39: note = subsp. lactis
GTWDDIGQGIGRVAYWVGKAMGNMSDVNQASRINRKKKH

SEQ ID NO: 347 - DNA, 117 bp, synthetic construct
  misc_feature 1..117: note = Polynucleotide encoding the polypeptide of SEQ IDNO: 346
  source 1..117: mol_type = other DNA
ggcacctgggatgatattggccagggcattggccgcgtggcgtattgggtgggcaaagcgatgggcaacatgagcgatgtgaaccaggcgagccgcattaaccgcaaaaaaaaacat

SEQ ID NO: 348 - protein, 35 aa, Lactococcus lactis
  source 1..35: note = subsp. lactis
KKWGWLAWVDPAYEFIKGFGKGAIKEGNKDKWKNI

SEQ ID NO: 349 - DNA, 105 bp, synthetic construct
  misc_feature 1..105: note = Polynucleotide encoding the polypeptide of SEQ IDNO: 348
  source 1..105: mol_type = other DNA
aaaaaatggggctggctggcgtgggtggatccggcgtatgaatttattaaaggctttggcaaaggcgcgattaaagaaggcaacaaagataaatggaaaaacatt

SEQ ID NO: 350 - protein, 19 aa, Streptomyces sp.
CVQSCSFGPLTWSCDGNTK

SEQ ID NO: 351 - DNA, 57 bp, synthetic construct
  misc_feature 1..57: note = Polynucleotide encoding the polypeptide of SEQ IDNO: 350
  source 1..57: mol_type = other DNA
tgcgtgcagagctgcagctttggcccgctgacctggagctgcgatggcaacaccaaa

SEQ ID NO: 352 - protein, 19 aa, Actinoplanes liguriae
SSGWVCTLTIECGTVICAC

SEQ ID NO: 353 - DNA, 57 bp, synthetic construct
  misc_feature 1..57: note = Polynucleotide encoding the polypeptide of SEQ IDNO: 352
  source 1..57: mol_type = other DNA
agcagcggctgggtgtgcaccctgaccattgaatgcggcaccgtgatttgcgcgtgc

SEQ ID NO: 354 - protein, 38 aa, Lactobacillus curvatus
  VARIANT 34: note = Xaa = any amino acid
  VARIANT 37: note = Xaa = any amino acid
YTAKQCLQAIGSCGIAGTGAGAAGGPAGAFVGAXVVXI

SEQ ID NO: 355 - DNA, 114 bp, synthetic construct
  misc_feature 1..114: note = Polynucleotide encoding the polypeptide of SEQ IDNO: 354
  misc_feature 100..102: note = nnn = is not TAA, TAG, or TGA
  misc_feature 109..111: note = nnn = is not TAA, TAG, or TGA
  source 1..114: mol_type = other DNA
  variation 100..111: note = n = A,T,C or G
tataccgcgaaacagtgcctgcaggcgattggcagctgcggcattgcgggcaccggcgcgggcgcggcgggcggcccggcgggcgcgtttgtgggcgcgnnngtggtgnnnatt

SEQ ID NO: 356 - protein, 42 aa, Lactobacillus sakei
  VARIANT 32: note = Xaa = any amino acid
TKYYGNGVYCNSKKCWVDWGQAAGGIGQTVVXGWLGGAIPGK

SEQ ID NO: 357 - DNA, 126 bp, synthetic construct
  misc_feature 1..126: note = Polynucleotide encoding the polypeptide of SEQ IDNO: 356
  misc_feature 94..96: note = nnn = is not TAA, TAG, or TGA
  source 1..126: mol_type = other DNA
accaaatattatggcaacggcgtgtattgcaacagcaaaaaatgctgggtggattggggccaggcggcgggcggcattggccagaccgtggtgnnnggctggctgggcggcgcgattccgggcaaa

SEQ ID NO: 358 - protein, 22 aa, Streptococcus mutans
FKSWSFCTPGCAKTGSFNSYCC

SEQ ID NO: 359 - DNA, 132 bp, synthetic construct
  misc_feature 1..132: note = Polynucleotide encoding the polypeptide of SEQ IDNO: 358
  source 1..132: mol_type = other DNA
tttaaaagctggagcttttgcaccccgggctgcgcgaaaaccggcagctttaacagctattgctgctttaaaagctggagcttttgcaccccgggctgcgcgaaaaccggcagctttaacagctattgctgc

SEQ ID NO: 360 - protein, 43 aa, Enterococcus mundtii
KYYGNGVSCNKKGCSVDWGKAIGIIGNNSAANLATGGAAGWSK

SEQ ID NO: 361 - DNA, 129 bp, synthetic construct
  misc_feature 1..129: note = Polynucleotide encoding the polypeptide of SEQ IDNO: 360
  source 1..129: mol_type = other DNA
aaatattatggcaacggcgtgagctgcaacaaaaaaggctgcagcgtggattggggcaaagcgattggcattattggcaacaacagcgcggcgaacctggcgaccggcggcgcggcgggctggagcaaa

SEQ ID NO: 362 - protein, 41 aa, Lactobacillus sakei
  VARIANT 9..37: note = X= any amino acid
KYYGNGVHXGKHSXTVDWGTAIGNIGNNAAANXATGXNAGG

SEQ ID NO: 363 - DNA, 123 bp, synthetic construct
  misc_feature 1..123: note = Polynucleotide encoding the polypeptide of SEQ IDNO: 362
  misc_feature 25..27: note = nnn = is not TAA, TAG, or TGA
  misc_feature 40..42: note = nnn = is not TAA, TAG, or TGA
  misc_feature 97..99: note = nnn = is not TAA, TAG, or TGA
  misc_feature 109..111: note = nnn = is not TAA, TAG, or TGA
  source 1..123: mol_type = other DNA
aaatattatggcaacggcgtgcatnnnggcaaacatagcnnnaccgtggattggggcaccgcgattggcaacattggcaacaacgcggcggcgaacnnngcgaccggcnnnaacgcgggcggc

SEQ ID NO: 364 - protein, 31 aa, Lactobacillus paracasei
GMSGYIQGIPDFLKGYLHGISAANKHKKGRL

SEQ ID NO: 365 - DNA, 93 bp, synthetic construct
  misc_feature 1..93: note = Polynucleotide encoding the polypeptide of SEQ IDNO: 364
  source 1..93: mol_type = other DNA
ggcatgagcggctatattcagggcattccggattttctgaaaggctatctgcatggcattagcgcggcgaacaaacataaaaaaggccgcctg

SEQ ID NO: 366 - protein, 31 aa, Leuconostoc mesenteroides
KGKGFWSWASKATSWLTGPQQPGSPLLKKHR

SEQ ID NO: 367 - DNA, 93 bp, synthetic construct
  misc_feature 1..93: note = Polynucleotide encoding the polypeptide of SEQ IDNO: 366
  source 1..93: mol_type = other DNA
aaaggcaaaggcttttggagctgggcgagcaaagcgaccagctggctgaccggcccgcagcagccgggcagcccgctgctgaaaaaacatcgc

SEQ ID NO: 368 - protein, 43 aa, Leuconostoc mesenteroides
KNYGNGVHCTKKGCSVDWGYAWTNIANNSVMNGLTGGNAGWHN

SEQ ID NO: 369 - DNA, 129 bp, synthetic construct
  misc_feature 1..129: note = Polynucleotide encoding the polypeptide of SEQ IDNO: 368
  source 1..129: mol_type = other DNA
aaaaactatggcaacggcgtgcattgcaccaaaaaaggctgcagcgtggattggggctatgcgtggaccaacattgcgaacaacagcgtgatgaacggcctgaccggcggcaacgcgggctggcataac

SEQ ID NO: 370 - protein, 47 aa, Bacillus subtilis
AIKLVQSPNGNFAASFVLDGTKWIFKSKYYDSSKGYWVGIYEVWDRK

SEQ ID NO: 371 - DNA, 141 bp, synthetic construct
  misc_feature 1..141: note = Polynucleotide encoding the polypeptide of SEQ IDNO: 370
  source 1..141: mol_type = other DNA
gcgattaaactggtgcagagcccgaacggcaactttgcggcgagctttgtgctggatggcaccaaatggatttttaaaagcaaatattatgatagcagcaaaggctattgggtgggcatttatgaagtgtgggatcgcaaa

SEQ ID NO: 372 - protein, 12 aa, Bacillus licheniformis
  VARIANT 7: note = Xaa = any amino acid
ISLEICXIFHDN

SEQ ID NO: 373 - DNA, 36 bp, synthetic construct
  misc_feature 1..36: note = Polynucleotide encoding the polypeptide of SEQ IDNO: 372
  misc_feature 19..21: note = nnn = is not TAA, TAG, or TGA
  source 1..36: mol_type = other DNA
attagcctggaaatttgcnnnatttttcatgataac

SEQ ID NO: 374 - protein, 37 aa, Lactococcus lactis
  source 1..37: note = subsp. lactis
TSYGNGVHCNKSKCWIDVSELETYKAGTVSNPKDILW

SEQ ID NO: 375 - DNA, 111 bp, synthetic construct
  misc_feature 1..111: note = Polynucleotide encoding the polypeptide of SEQ IDNO: 374
  source 1..111: mol_type = other DNA
accagctatggcaacggcgtgcattgcaacaaaagcaaatgctggattgatgtgagcgaactggaaacctataaagcgggcaccgtgagcaacccgaaagatattctgtgg

SEQ ID NO: 376 - protein, 9 aa, Serratia plymuthica
DYHHGVRVL

SEQ ID NO: 377 - DNA, 27 bp, synthetic construct
  misc_feature 1..27: note = Polynucleotide encoding the polypeptide of SEQ IDNO: 376
  source 1..27: mol_type = other DNA
gattatcatcatggcgtgcgcgtgctg

SEQ ID NO: 378 - protein, 15 aa, Halobacterium sp.
DIDITGCSACKYAAG

SEQ ID NO: 379 - DNA, 45 bp, synthetic construct
  misc_feature 1..45: note = Polynucleotide encoding the polypeptide of SEQ IDNO: 378
  source 1..45: mol_type = other DNA
gatattgatattaccggctgcagcgcgtgcaaatatgcggcgggc

SEQ ID NO: 380 - protein, 12 aa, Bacillus subtilis
  VARIANT 1..6: note = X= any amino acid
XXKEIXHIFHDN

SEQ ID NO: 381 - DNA, 36 bp, synthetic construct
  misc_feature 1..36: note = Polynucleotide encoding the polypeptide of SEQ IDNO: 380
  misc_feature 1..3: note = nnn = is not TAA, TAG, or TGA
  misc_feature 4..6: note = nnn = is not TAA, TAG, or TGA
  misc_feature 16..18: note = nnn = is not TAA, TAG, or TGA
  source 1..36: mol_type = other DNA
nnnnnnaaagaaattnnncatatttttcatgataac

SEQ ID NO: 382 - protein, 12 aa, Lactobacillus curvatus
TPVVNPPFLQQT

SEQ ID NO: 383 - DNA, 36 bp, synthetic construct
  misc_feature 1..36: note = Polynucleotide encoding the polypeptide of SEQ IDNO: 382
  source 1..36: mol_type = other DNA
accccggtggtgaacccgccgtttctgcagcagacc

SEQ ID NO: 384 - protein, 10 aa, Lactobacillus curvatus
  VARIANT 10: note = Xaa = any amino acid
VAPFPEQFLX

SEQ ID NO: 385 - DNA, 30 bp, synthetic construct
  misc_feature 1..30: note = Polynucleotide encoding the polypeptide of SEQ IDNO: 384
  misc_feature 28..30: note = nnn = is not TAA, TAG, or TGA
  source 1..30: mol_type = other DNA
gtggcgccgtttccggaacagtttctgnnn

SEQ ID NO: 386 - protein, 9 aa, Lactobacillus curvatus
NIPQLTPTP

SEQ ID NO: 387 - DNA, 27 bp, synthetic construct
  misc_feature 1..27: note = Polynucleotide encoding the polypeptide of SEQ IDNO: 386
  source 1..27: mol_type = other DNA
aacattccgcagctgaccccgaccccg

SEQ ID NO: 388 - protein, 18 aa, Bacillus thuringiensis
  VARIANT 4..10: note = X= any amino acid
  source 1..18: note = subsp. entomocidus
DWTXWSXLVXAACSVELL

SEQ ID NO: 389 - DNA, 54 bp, synthetic construct
  misc_feature 1..54: note = Polynucleotide encoding the polypeptide of SEQ IDNO: 388
  misc_feature 10..12: note = nnn = is not TAA, TAG, or TGA
  misc_feature 19..21: note = nnn = is not TAA, TAG, or TGA
  misc_feature 28..30: note = nnn = is not TAA, TAG, or TGA
  source 1..54: mol_type = other DNA
gattggaccnnntggagcnnnctggtgnnngcggcgtgcagcgtggaactgctg

SEQ ID NO: 390 - protein, 30 aa, Lactobacillus curvatus
AYPGNGVHCGKYSCTVDKQTAIGNIGNNAA

SEQ ID NO: 391 - DNA, 90 bp, synthetic construct
  misc_feature 1..90: note = Polynucleotide encoding the polypeptide of SEQ IDNO: 390
  source 1..90: mol_type = other DNA
gcgtatccgggcaacggcgtgcattgcggcaaatatagctgcaccgtggataaacagaccgcgattggcaacattggcaacaacgcggcg

SEQ ID NO: 392 - protein, 43 aa, Carnobacterium divergens
TKYYGNGVYCNSKKCWVDWGTAQGCIDVVIGQLGGGIPGKGKC

SEQ ID NO: 393 - DNA, 129 bp, synthetic construct
  misc_feature 1..129: note = Polynucleotide encoding the polypeptide of SEQ IDNO: 392
  source 1..129: mol_type = other DNA
accaaatattatggcaacggcgtgtattgcaacagcaaaaaatgctgggtggattggggcaccgcgcagggctgcattgatgtggtgattggccagctgggcggcggcattccgggcaaaggcaaatgc

SEQ ID NO: 394 - protein, 62 aa, Enterococcus sp.
NRWYCNSAAGGVGGAAVCGLAGYVGEAKENIAGEVRKGWGMAGGFTHNKACKSFPGSGWASG

SEQ ID NO: 395 - DNA, 186 bp, synthetic construct
  misc_feature 1..186: note = Polynucleotide encoding the polypeptide of SEQ IDNO: 394
  source 1..186: mol_type = other DNA
aaccgctggtattgcaacagcgcggcgggcggcgtgggcggcgcggcggtgtgcggcctggcgggctatgtgggcgaagcgaaagaaaacattgcgggcgaagtgcgcaaaggctggggcatggcgggcggctttacccataacaaagcgtgcaaaagctttccgggcagcggctgggcgagcggc

SEQ ID NO: 396 - protein, 39 aa, Enterococcus faecium
TTKNYGNGVCNSVNWCQCGNVWASCNLATGCAAWLCKLA

SEQ ID NO: 397 - DNA, 117 bp, synthetic construct
  misc_feature 1..117: note = Polynucleotide encoding the polypeptide of SEQ IDNO: 396
  source 1..117: mol_type = other DNA
accaccaaaaactatggcaacggcgtgtgcaacagcgtgaactggtgccagtgcggcaacgtgtgggcgagctgcaacctggcgaccggctgcgcggcgtggctgtgcaaactggcg

SEQ ID NO: 398 - protein, 30 aa, Paenibacillus polymyxa
ASIIKTTIKVSKAVCKTLTCICTGSCSNCK

SEQ ID NO: 399 - DNA, 90 bp, synthetic construct
  misc_feature 1..90: note = Polynucleotide encoding the polypeptide of SEQ IDNO: 398
  source 1..90: mol_type = other DNA
gcgagcattattaaaaccaccattaaagtgagcaaagcggtgtgcaaaaccctgacctgcatttgcaccggcagctgcagcaactgcaaa

SEQ ID NO: 400 - protein, 31 aa, Staphylococcus epidermidis
SASIVKTTIKASKKLCRGFTLTCGCHFTGKK

SEQ ID NO: 401 - DNA, 93 bp, synthetic construct
  misc_feature 1..93: note = Polynucleotide encoding the polypeptide of SEQ IDNO: 400
  source 1..93: mol_type = other DNA
agcgcgagcattgtgaaaaccaccattaaagcgagcaaaaaactgtgccgcggctttaccctgacctgcggctgccattttaccggcaaaaaa

SEQ ID NO: 402 - protein, 43 aa, Enterococcus faecium
KYYGNGVSCNKKGCSVDWGKAIGIIGNNAAANLTTGGKAAWAC

SEQ ID NO: 403 - DNA, 129 bp, synthetic construct
  misc_feature 1..129: note = Polynucleotide encoding the polypeptide of SEQ IDNO: 402
  source 1..129: mol_type = other DNA
aaatattatggcaacggcgtgagctgcaacaaaaaaggctgcagcgtggattggggcaaagcgattggcattattggcaacaacgcggcggcgaacctgaccaccggcggcaaagcggcgtgggcgtgc

SEQ ID NO: 404 - protein, 39 aa, Paenibacillus polymyxa
ATYYGNGLYCNKQKHYTWVDWNKASREIGKITVNGWVQH

SEQ ID NO: 405 - DNA, 117 bp, synthetic construct
  misc_feature 1..117: note = Polynucleotide encoding the polypeptide of SEQ IDNO: 404
  source 1..117: mol_type = other DNA
gcgacctattatggcaacggcctgtattgcaacaaacagaaacattatacctgggtggattggaacaaagcgagccgcgaaattggcaaaattaccgtgaacggctgggtgcagcat

SEQ ID NO: 406 - protein, 35 aa, Bacillus circulans
VNYGNGVSCSKTKCSVNWGIITHQAFRVTSGVASG

SEQ ID NO: 407 - DNA, 105 bp, synthetic construct
  misc_feature 1..105: note = Polynucleotide encoding the polypeptide of SEQ IDNO: 406
  source 1..105: mol_type = other DNA
gtgaactatggcaacggcgtgagctgcagcaaaaccaaatgcagcgtgaactggggcattattacccatcaggcgtttcgcgtgaccagcggcgtggcgagcggc

SEQ ID NO: 408 - protein, 30 aa, Paenibacillus polymyxa
FVYGNGVTSILVQAQFLVNGQRRFFYTPDK

SEQ ID NO: 409 - DNA, 90 bp, synthetic construct
  misc_feature 1..90: note = Polynucleotide encoding the polypeptide of SEQ IDNO: 408
  source 1..90: mol_type = other DNA
tttgtgtatggcaacggcgtgaccagcattctggtgcaggcgcagtttctggtgaacggccagcgccgctttttttataccccggataaa

SEQ ID NO: 410 - protein, 13 aa, Lactobacillus rhamnosus
AVPAVRKTNETLD

SEQ ID NO: 411 - DNA, 39 bp, synthetic construct
  misc_feature 1..39: note = Polynucleotide encoding the polypeptide of SEQ IDNO: 410
  source 1..39: mol_type = other DNA
gcggtgccggcggtgcgcaaaaccaacgaaaccctggat

SEQ ID NO: 412 - protein, 69 aa, Bacillus licheniformis
MKNSAAREAFKGANHPAGMVSEEELKALVGGNDVNPETTPATTSSWTCITAGVTVSASLCPTTKCTSRC

SEQ ID NO: 413 - DNA, 207 bp, synthetic construct
  misc_feature 1..207: note = Polynucleotide encoding the polypeptide of SEQ IDNO: 412
  source 1..207: mol_type = other DNA
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgcccgaccaccaaatgcaccagccgctgc

SEQ ID NO: 414 - protein, 36 aa, Lactobacillus plantarum
KYYGNGLSCSKKGCTVNWGQAFSCGVNRVATAGHGK

SEQ ID NO: 415 - DNA, 108 bp, synthetic construct
  misc_feature 1..108: note = Polynucleotide encoding the polypeptide of SEQ IDNO: 414
  source 1..108: mol_type = other DNA
aaatattatggcaacggcctgagctgcagcaaaaaaggctgcaccgtgaactggggccaggcgtttagctgcggcgtgaaccgcgtggcgaccgcgggccatggcaaa

SEQ ID NO: 416 - protein, 24 aa, Lactobacillus acidophilus
GNPKVAHCASQIGRSTAWGAVSGA

SEQ ID NO: 417 - DNA, 72 bp, synthetic construct
  misc_feature 1..72: note = Polynucleotide encoding the polypeptide of SEQ IDNO: 416
  source 1..72: mol_type = other DNA
ggcaacccgaaagtggcgcattgcgcgagccagattggccgcagcaccgcgtggggcgcggtgagcggcgcg

SEQ ID NO: 418 - protein, 40 aa, Enterococcus faecalis; identical to SEQ ID NO: 1

SEQ ID NO: 419 - DNA, 120 bp, synthetic construct
  misc_feature 1..120: note = Polynucleotide encoding the polypeptide of SEQ IDNO: 418
  source 1..120: mol_type = other DNA
tggctgccgccggcgggcctgctgggccgctgcggccgctggtttcgcccgtggctgctgtggctgcagagcggcgcgcagtataaatggctgggcaacctgtttggcctgggcccgaaa

SEQ ID NO: 420 - protein, 40 aa, Anabaena variabilis
NLDQWLTEQVHEFQDMYLEPQAISNQDITFKLSDLDFIHN

SEQ ID NO: 421 - DNA, 124 bp, Anabaena variabilis
  source 1..124: mol_type = genomic DNA
taatttagatcagtggttaacagaacaagttcatgagtttcaagatatgtacttggaaccacaagcaatatccaatcaagacattaccttcaaactatctgacctagattttattcataattga

SEQ ID NO: 422 - protein, 40 aa, Nostoc sp.; identical to SEQ ID NO: 420

SEQ ID NO: 423 - DNA, 123 bp, Nostoc sp.
  source 1..123: mol_type = genomic DNA
aatttagatcaatggttaacagaacaagttcatgagtttcaagatatgtacttggaaccacaagcaatatccaatcaagacattaccttcaaactgtcagacctagattttattcataattga

SEQ ID NO: 424 - protein, 7 aa, Nostoc azollae
HREKKSA

SEQ ID NO: 425 - DNA, 24 bp, Nostoc azollae
  source 1..24: mol_type = genomic DNA
cacagagagaaaaaatcagcatag

SEQ ID NO: 426 - protein, 23 aa, Acaryochloris marina
TSNNWLAKNYLSMWNKKSSNPNL

SEQ ID NO: 427 - DNA, 72 bp, Acaryochloris marina
  source 1..72: mol_type = genomic DNA
acaagcaataactggctagccaaaaactatctttctatgtggaataaaaagagcagtaatccaaacctttag

SEQ ID NO: 428 - protein, 6 aa, Cyanothece sp.
FRYFWW

SEQ ID NO: 429 - DNA, 21 bp, Cyanothece sp.
  source 1..21: mol_type = genomic DNA
tttagatatttttggtggtaa

SEQ ID NO: 430 - protein, 6 aa, Cyanothece sp.
FRYFWW

SEQ ID NO: 431 - DNA, 21 bp, Cyanothece sp.
  source 1..21: mol_type = genomic DNA
tttagatatttttggtggtaa

SEQ ID NO: 432 - protein, 9 aa, Cyanothece sp.
CGEKWRIFS

SEQ ID NO: 433 - DNA, 27 bp, Cyanothece sp.
  source 1..27: mol_type = genomic DNA
tgtggagaaaaatggagaatttttagc

SEQ ID NO: 434 - protein, 8 aa, Cyanothece sp.
FRLQLWQF

SEQ ID NO: 435 - DNA, 24 bp, Cyanothece sp.
  source 1..24: mol_type = genomic DNA
tttcgcttacaactgtggcaattt

SEQ ID NO: 436 - protein, 16 aa, Cyanothece sp.
LGCNQSSIWSIFFWNH

SEQ ID NO: 437 - DNA, 51 bp, Cyanothece sp.
  source 1..51: mol_type = genomic DNA
ctaggatgtaaccagagcagtatctggtcaatttttttctggaatcattaa

SEQ ID NO: 438 - protein, 40 aa, Microcoleus chthonoplastes
YNLQGLPAIESEDCIPDSVAPSDDWFSGVSSLFNRLTGLG

SEQ ID NO: 439 - DNA, 123 bp, Microcoleus chthonoplastes
  source 1..123: mol_type = genomic DNA
tataacctacaggggttgccagcaattgagtcagaagactgtatcccagattctgtagcgccttcggatgattggttttcaggcgtatcgtctctgtttaaccgcttgactgggttgggttag

SEQ ID NO: 440 - protein, 37 aa, Nostoc sp.
WMAIRRILRCHPFHPGGYDPVPELGEHCCHHDSGNKG

SEQ ID NO: 441 - DNA, 114 bp, Nostoc sp.
  source 1..114: mol_type = genomic DNA
tggatggcgattcgccgcattttgcgttgtcatccattccacccagggggttatgatcctgtaccagagttgggtgagcattgttgtcatcatgatagcgggaataaggggtga

SEQ ID NO: 442 - protein, 35 aa, Anabaena variabilis
WMGIRRILRCHPFHPGGYDPVPEVGEHCCHHDSGK

SEQ ID NO: 443 - DNA, 108 bp, Anabaena variabilis
  source 1..108: mol_type = genomic DNA
tggatggggattcgccgcattttgcgttgtcatccattccacccaggcggttatgatcctgtaccagaggtgggtgagcattgttgtcatcatgatagcgggaagtag

SEQ ID NO: 444 - protein, 48 aa, Nodularia spumigena
WMATRRILRCHPFHPGGYDPVPEVKHNCCDQHLSDSGKQTTEDHHKGS

SEQ ID NO: 445 - DNA, 147 bp, Nodularia spumigena
  source 1..147: mol_type = genomic DNA
tggatggcgactcggcggattttgcgttgtcatcccttccatcctggtggatatgatccagttccagaggtaaaacacaattgctgcgatcagcatctgtccgattctgggaaacagaccacagaagaccatcacaaaggctcgtag

SEQ ID NO: 446 - protein, 34 aa, Nostoc azollae
WMATLRILRCHPFHPGGYDPVPGLAEKSCCDHHD

SEQ ID NO: 447 - DNA, 105 bp, Nostoc azollae
  source 1..105: mol_type = genomic DNA
tggatggcaactttgcggattttacgctgtcatcctttccatcctggtggttatgatcctgtaccaggactagcggaaaaatcctgttgtgaccatcatgattga

SEQ ID NO: 448 - protein, 28 aa, Synechococcus sp
WLTAKRFCRCHPLHPGGYDPVPEKKSVL

SEQ ID NO: 449 - DNA, 87 bp, Synechococcus sp
  source 1..87: mol_type = genomic DNA
tggctaacagccaagcgcttttgtcgctgtcatccgcttcatcctggcgggtatgatccggtaccggagaagaaatcggtactctaa

SEQ ID NO: 450 - protein, 23 aa, Prochlorococcus marinus
WLTLRRLSRCHPFTPCGCDPVPD

SEQ ID NO: 451 - DNA, 72 bp, Prochlorococcus marinus
  source 1..72: mol_type = genomic DNA
tggctcaccctgcggcgcctgtctcgttgccatccttttaccccctgtggttgcgacccggtgcctgattaa

SEQ ID NO: 452 - protein, 69 aa, Escherichia coli
MSYKKLYQLTAIFSLPLTILLVSLSSLRIVGEGNSYVDVFLSFIIFLGFIELIHGIRKILVWSGWKNGS

SEQ ID NO: 453 - DNA, 210 bp, Escherichia coli
  source 1..210: mol_type = genomic DNA
atgagttataaaaaactgtaccaattgacggctatatttagtttacctcttactatcttattggtttcactttcatcccttcggattgttggcgaagggaattcttatgttgacgtttttctaagctttataatatttcttggttttattgagctgattcatgggattcgaaagattttggtctggtcaggctggaaaaacggaagttaa

SEQ ID NO: 454 - protein, 85 aa, Escherichia coli
MGLKLDLTWFDKSTEDFKGEEYSKDFGDDGSVMESLGVPFKDNVNNGCFDVIAEWVPLLQPYFNHQIDISDNEYFVSFDYRDGDW

SEQ ID NO: 455 - DNA, 258 bp, Escherichia coli
  source 1..258: mol_type = genomic DNA
atgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtga

SEQ ID NO: 456 - protein, 113 aa, Escherichia coli
MSLRYYIKNILFGLYCTLIYIYLITKNSEGYYFLVSDKMLYAIVISTILCPYSKYAIEYIAFNFIKKDFFERRKNLNNAPVAKLNLFMLYNLLCLVLAIPFGLLGLFISIKNN

SEQ ID NO: 457 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagggtattatttccttgtgtcagataagatgctatatgcaatagtgataagcactattctatgtccatattcaaaatatgctattgaatacatagcttttaacttcataaagaaagattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 458 - protein, 85 aa, Escherichia coli
MGLKLHIHWFDKKTEEFKGGEYSKDFGDDGSVIESLGMPLKDNINNGWFDVEKPWVSILQPHFKNVIDISKFDYFVSFVYRDGNW

SEQ ID NO: 459 - DNA, 258 bp, Escherichia coli
  source 1..258: mol_type = genomic DNA
atggggcttaaattacatattcattggtttgataagaaaaccgaagagtttaaaggcggtgaatactcaaaagacttcggtgatgatggttctgtcattgaaagtctggggatgcctttaaaggataatattaataatggttggtttgatgttgaaaaaccatgggtttcgatattacagccacactttaaaaatgtaatcgatattagtaaatttgattactttgtatcctttgtttaccgggatggtaactggtaa

SEQ ID NO: 460 - protein, 86 aa, Escherichia coli
MELKHSISDYTEAEFLEFVKKICRAEGATEEDDNKLVREFERLTEHPDGSDLIYYPRDDREDSPEGIVKEIKEWRAANGKSGFKQG

SEQ ID NO: 461 - DNA, 261 bp, Escherichia coli
  source 1..261: mol_type = genomic DNA
atggaactgaaacatagtattagtgattataccgaggctgaatttctggagtttgtaaaaaaaatatgtagagctgaaggtgctactgaagaggatgacaataaattagtgagagagtttgagcgattaactgagcacccagatggttcagatctgatttattatcctcgcgatgacagggaagatagtcctgaagggattgtcaaggaaattaaagaatggcgagctgctaacggtaagtcaggatttaaacagggctga

SEQ ID NO: 462 - protein, 178 aa, Citrobacter freundii
MMNEHSIDTDNRKANNALYLFIIIGLIPLLCIFVVYYKTPDALLLRKIATSTENLPSITSSYNPLMTKVMDIYCKTAPFLALILYILTFKIRKLINNTDRNTVLRSCLLSPLVYAAIVYLFCFRNFELTTAGRPVRLMATNDATLLLFYIGLYSIIFFTTYITLFTPVTAFKLLKKRQ

SEQ ID NO: 463 - DNA, 537 bp, Citrobacter freundii
  source 1..537: mol_type = genomic DNA
atgatgaatgaacactcaatagatacggacaacagaaaggccaataacgcattgtatttatttataataatcggattaataccattattgtgcatttttgttgtttactacaaaacgccagacgctttacttttacgtaaaattgctacaagcactgagaatctcccgtcaataacatcctcctacaacccattaatgacaaaggttatggatatttattgtaaaacagcgcctttccttgccttaatactatacatcctaacctttaaaatcagaaaattaatcaacaacaccgacaggaacactgtacttagatcttgtttattaagtccattggtctatgcagcaattgtttatctattctgcttccgaaattttgagttaacaacagccggaaggcctgtcagattaatggccaccaatgacgcaacactattgttattttatattggtctgtactcaataattttctttacaacctatatcacgctattcacaccagtcactgcatttaaattattaaaaaaaaggcagtaa

SEQ ID NO: 464 - protein, 111 aa, Escherichia coli
MNRKYYFNNMWWGWVTGGYMLYMSWDYEFKYRLLFWCISLCGMVLYPVAKWYIEDTALKFTRPDFWNSGFFADTPGKMGLLAVYTGTVFILSLPLSMIYILSVIIKRLSVR

SEQ ID NO: 465 - DNA, 336 bp, Escherichia coli
  source 1..336: mol_type = genomic DNA
atgaacagaaaatattattttaataatatgtggtggggatgggtgacggggggatatatgctgtatatgtcatgggattatgagtttaaatacagattactgttctggtgtatttctctctgcggaatggttttgtatccggttgcaaaatggtatattgaagatacagctctaaaatttacccggcctgatttctggaacagcggtttttttgctgatacacctggaaaaatggggttgcttgcggtttatacgggtactgttttcatattatctcttccgttaagtatgatatatattctttctgttattataaaaaggctgtctgtaagatag

SEQ ID NO: 466 - protein, 115 aa, Escherichia coli
MKLDISVKYLLKSLIPILIILTVFYLGWKDNQENARMFYAFIGCIISAITFPFSMRIIQKMVIRFTGKEFWQKDFFTNPVGGSLTAIFELFCFVISVPVVAIYLIFILCKALSGK

SEQ ID NO: 467 - DNA, 348 bp, Escherichia coli
  source 1..348: mol_type = genomic DNA
atgaaactggatatatctgtaaagtatttactgaaaagcctgataccaatcctcattattcttacagttttttatctgggatggaaagataaccaggaaaatgcaagaatgttttatgcgttcatcggatgcattatcagtgccattacttttcctttttcaatgaggataatacagaaaatggtaataaggtttacagggaaagaattctggcaaaaagacttctttacaaatccagttggcggaagcttaactgcaatatttgaattattctgtttcgttatatcagttcctgtggttgccatttacttaatttttatactctgcaaagccctttcaggaaaatga

SEQ ID NO: 468 - protein, 131 aa, Escherichia coli
MHNTLLEKIIAYLSLPGFHSLNNPPLSEAFNLYVHTAPLAATSLFIFTHKELELKPKSSPLRALKILTPFTILYISMIYCFLLTDTELTLSSKTFVLIVKKRSVFVFFLYNTIYWDIYIHIFVLLVPYRNI

SEQ ID NO: 469 - DNA, 396 bp, Escherichia coli
  source 1..396: mol_type = genomic DNA
atgcacaatacactcctcgaaaaaatcatcgcatacctatccctaccaggatttcattcattaaacaacccgcccctaagcgaagcattcaatctctatgttcatacagcccctttagctgcaaccagcttattcatattcacacacaaagaattagagttaaaaccaaagtcgtcacctctgcgggcactaaagatattaactcctttcactattctttatatatccatgatatactgtttcttgctaactgacacagaactaaccttgtcatcaaaaacatttgtattaatagtcaaaaaacgatctgtttttgtcttttttctatataacactatatattgggatatatatattcacatatttgtacttttggttccttataggaacatataa

SEQ ID NO: 470 - protein, 85 aa, Escherichia coli
MELKNSISDYTETEFKKIIEDIINCEGDEKKQDDNLEHFISVTEHPSGSDLIYYPEGNNDGSPEAVIKEIKEWRAANGKSGFKQG

SEQ ID NO: 471 - DNA, 258 bp, Escherichia coli
  source 1..258: mol_type = genomic DNA
atggaactgaaaaacagcattagtgattacactgaaactgaattcaaaaaaattattgaagacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 472 - protein, 98 aa, Lactococcus lactis
  source 1..98: note = subsp. lactis
MKKKQIEFENELRSMLATALEKDISQEERNALNIAEKALDNSEYLPKIILNLRKALTPLAINRTLNHDLSELYKFITSSKASNKNLGGGLIMSWGRLF

SEQ ID NO: 473 - DNA, 297 bp, Lactococcus lactis
  source 1..297: mol_type = genomic DNA; note = subsp. lactis
atgaaaaaaaaacaaatagaatttgaaaacgagctaagaagtatgttggctaccgcccttgaaaaagacattagtcaagaggaaagaaatgctctgaatattgcagaaaaggcgcttgacaattctgaatatttaccaaaaattattttaaacctcagaaaagccctaactccattagctataaatcgaacacttaaccatgatttatctgaactgtataaattcattacaagttccaaagcatcaaacaaaaatttaggtggtggtttaattatgtcgtggggacgactattctaa

SEQ ID NO: 474 - protein, 98 aa, Lactococcus lactis; identical to SEQ ID NO: 472
  source 1..98: note = subsp. cremoris

SEQ ID NO: 475 - DNA, 297 bp, Lactococcus lactis; identical to SEQ ID NO: 473
  source 1..297: mol_type = genomic DNA; note = subsp. cremoris

SEQ ID NO: 476 - protein, 87 aa, Escherichia coli
MNKMAMIDLAKLFLASKITAIEFSERICVERRRLYGVKDLSPNILNCGEELFMAAERFEPDADRANYEIDDNGLKVEVRSILEKFKL

SEQ ID NO: 477 - DNA, 249 bp, Escherichia coli
  source 1..249: mol_type = genomic DNA
atgatcgatttggcgaaattatttttagcttcgaaaattacagtgattgagttttcagagcgaatttgtgttgaacggagaagattgtatggtgttaaggatttgtctccgaatatattaaattgtggggaagagttgtctatggctgctgagcgatttgagcctgatgcagatagggctaattatgaaattgatgataatggacttaaggtcgaggtccgatctatcttggaaaaacttaaatcataa

SEQ ID NO: 478 - protein, 83 aa, Escherichia coli
MKLSPKAAIEVCNEAAKKGLWILGIDGGHWLNPGFRIDSSASWTYDMPEEYKSKIPENNRLAIENIKDDIENGYTAFIITLKM

SEQ ID NO: 479 - DNA, 243 bp, Escherichia coli
  source 1..243: mol_type = genomic DNA
atgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggagaatacaaatcaaaaatccctgaaaataatagattggctattgaaaatattaaagatgatattgagaatggatacactgctttcattatcacgttaa

SEQ ID NO: 480 - protein, 85 aa, Escherichia coli
MGLKLHINWFDKRTEEFKGGEYSKDFGDDGSVIERLGMPFKDNINNGWFDVIAEWVPLLQPYFNHQIDISDNEYFVSFDYRDGDW

SEQ ID NO: 481 - DNA, 258 bp, Escherichia coli
  source 1..258: mol_type = genomic DNA
atggggcttaaattacatattaattggtttgataagacgaccgaggaatttaaaggtggtgagtattcaaaagattttggagatgatggctcggtcattgaacgtcttggaatgcctttaaaagataatatcaataatggttggtttgatgttatagctgaatgggtacctttgctacaaccatactttaatcatcaaattgatatttccgataatgagtattttgtttcgtttgattatcgtgatggtgattggtga

SEQ ID NO: 482 - protein, 85 aa, Escherichia coli
MELKKSIGDYTETEFKKIIENIINCEGDEKKQDDNLEHFISVTEHPSGSDLIYYPEGNNDGSPEAVIKEIKEWRAANGKSGFKQG

SEQ ID NO: 483 - DNA, 258 bp, Escherichia coli
  source 1..258: mol_type = genomic DNA
gtggagctaaagaaaagtattggtgattacactgaaaccgaattcaaaaaaattattgaaaacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 484 - protein, 86 aa, Escherichia coli [sequence omitted]

SEQ ID NO: 485 - DNA, 261 bp, Escherichia coli
  source 1..261: mol_type = genomic DNA
atggaactgaagcatagcattagtgattatacagaagctgaatttttacaacttgtaacaacaatttgtaatgcgaacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggctaa

SEQ ID NO: 486 - protein, 141 aa, Escherichia coli
MLTLYGYIRNVFLYRMNDRSCGDFMKVISMKFIFILTIIALAAVFFWSEDKGPACYQVSDEQARTFVKNDYLQRMKRWDNDVQLLGTEIPKITWEKIERSLTDVEDEKTLLVPFKAEGPDGKRMYYGMYHCEEGYVEYAND

SEQ ID NO: 487 - DNA, 354 bp, Escherichia coli
  source 1..354: mol_type = genomic DNA
atgaaagtaattagcatgaaatttatttttattttaacgattattgctcttgctgctgtttttttctggtctgaagataaaggtccggcatgctatcaggtcagcgatgaacaggccagaacgtttgtaaaaaatgattacctgcaaagaatgaaacgctgggacaacgatgtacaacttcttggtacagaaatcccgaaaattacatgggaaaagattgagagaagtttaacagatgttgaagatgaaaaaacacttcttgtcccatttaaagctgaaggcccggacggtaagagaatgtattatggcatgtaccattgtgaggagggatatgttgaatatgcgaatgactaa

SEQ ID NO: 488 - protein, 175 aa, Escherichia coli
MTSNKDKNKKANEILYAFSIIGIIPLMAILILRINDPYSQVLYYLYNKVAFLPSITSLHDPVMTTLMSNYNKTAPVMGILVFLCTYKTREIIKPVTRKLVVQSCFWGPVFYAILIYITLFYNLELTTAGGFFKLLSHNVITLFILYCSIYFTVLTMTYAILLMPLLVIKYFKGRQ

SEQ ID NO: 489 - DNA, 528 bp, Escherichia coli
  source 1..528: mol_type = genomic DNA
atgaccagcaataaagataagaacaagaaagcaaacgaaatattatatgcattttccataatcgggattattccattaatggctatattaatacttcgaataaatgatccatattctcaagtgctgtactacttatataataaggtggcatttctcccttctattacatcattgcatgatcccgtcatgacaacacttatgtcaaactacaacaagacagcgccagttatgggtattctcgtttttctttgcacatataagacaagagaaatcataaagccagtaacaagaaaacttgttgtgcaatcctgtttctgggggcccgttttttatgccattctgatttatatcacactgttctataatctggaactaacaacagcaggtggtttttttaaattattatctcataatgtcatcactctgtttattttatattgctccatttactttactgttttaaccatgacatatgcgattttactgatgccattacttgtcattaaatattttaaagggaggcagtaa

SEQ ID NO: 490 - protein, 78 aa, Escherichia coli
MDRKRTKLELLFAFIINATAIYIALAIYDCVFRGKDFLSMHTFCFSALMSAICYFVGDNYYSISDKIKRRSYENSDSK

SEQ ID NO: 491 - DNA, 237 bp, Escherichia coli
  source 1..237: mol_type = genomic DNA
atggatagaaaaagaacaaaattagagttgttatttgcatttataataaatgccaccgcaatatatattgcattagctatatatgattgtgtttttagaggaaaggactttttatccatgcatacattttgcttctctgcattaatgtctgcaatatgttactttgttggtgataattattattcaatatccgataagataaaaaggagatcatatgagaactctgactctaaatga

SEQ ID NO: 492 - protein, 113 aa, Shigella sonnei
MSLRYYIKNILFGLYCALIYIYLITKNNEGYYFLASDKMLYAIVISTILCPYSKYAIEHIFFKFIKKDFFRKRKNLNKCPRGKIKPYLCVYNLLCLVLAIPFGLLGLVYINKE

SEQ ID NO: 493 - DNA, 342 bp, Shigella sonnei
  source 1..342: mol_type = genomic DNA
atgagtttaagatactacataaaaaatattttgtttggcctatactgcgcacttatatatatataccttataacaaaaaacaacgaagggtattatttcctagcgtcagataagatgctatacgcaatagtgataagcactattctatgcccatattcaaaatatgctattgaacacatattttttaagttcataaagaaagattttttcagaaaaagaaaaaacctaaataaatgcccccgtggcaaaattaaaccgtatttatgcgtatacaatctactttgtttggtcctagcaatcccatttggattgctaggacttgtttatatcaataaagaataa

SEQ ID NO: 494 - protein, 113 aa, Escherichia coli
MSLRYYIKNILFGLYCTLIYIYLITKNSEEYYFLVTDKMLYAIVISTILCPYSKYAIEHIAFNFIKKHFFERRKNLNNAPVAKLNLFMLYNLLCLVLAIPFGLLGLFISIKNN

SEQ ID NO: 495 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagagtattatttccttgtgacagataagatgctatatgcaatagtgataagcactattctatgtccatattcaaaatatgctattgaacacatagcttttaacttcataaagaaacattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 496 - protein, 113 aa, Leuconostoc gelidum
MRKNNILLDDAKIYTNKLYLLLIDRKDDAGYGDICDVLFQVSKKLDSTKNVEALINRLVNYIRITASTNRIKFSKDEEAVIIELGVIGQKAGLNGQYMADFSDKSQFYSIFER

SEQ ID NO: 497 - DNA, 342 bp, Leuconostoc gelidum
  source 1..342: mol_type = genomic DNA
ttgagaaaaaataacattttattggacgatgctaaaatatacacgaacaaactctatttgctattaatcgatagaaaagatgacgctgggtatggagatatttgtgatgttttgtttcaggtatccaaaaaattagatagcacaaaaaatgtagaagcattgattaaccgattggtcaattatatacgaattaccgcttcaacaaacagaattaagttttcaaaagatgaagaggctgtaattatagaacttggtgtaattggtcagaaggctggattaaacggccaatacatggctgatttttctgacaaatctcagttttatagtatctttgaaagataa

SEQ ID NO: 498 - protein, 91 aa, Lactococcus lactis
  source 1..91: note = subsp. cremoris
MKKKVDTEKQITSWASDLASKNETKVQEKLILSSYIQDIENHVYFPKAMISLEKKLRDQNNICALSKEVNQFYFKVVEVNQRKSWMVGLIV

SEQ ID NO: 499 - DNA, 276 bp, Lactococcus lactis
  source 1..276: mol_type = genomic DNA; note = subsp. cremoris
atgaaaaaaaaagttgatacagaaaaacaaattacttcttgggcatctgacttagcttccaaaaatgaaacaaaggttcaagaaaaattaatactgtcttcttatattcaggacatcgaaaaccatgtttactttccaaaagcaatgatttctttagaaaaaaaattacgagaccaaaataatatttgcgctttatcaaaagaagtcaatcagttttattttaaagttgttgaagtaaatcaaagaaaatcctggatggtaggtttgatagtttaa

SEQ ID NO: 500 - protein, 112 aa, Pediococcus acidilactici
MNKTKSEHIKQQALDLFTRLQFLLQKHDTIEPYQYVLDILETGISKTKHNQQTPERQARVVYNKIASQALVDKLHFTAEENKVLAAINELAHSQKGWGEFNMLDTTNTWPSQ

SEQ ID NO: 501 - DNA, 339 bp, Pediococcus acidilactici
  source 1..339: mol_type = genomic DNA
atgaataagactaagtcggaacatattaaacaacaagctttggacttatttactaggctacagtttttactacagaagcacgatactatcgaaccttaccagtacgttttagatattctggagactggtatcagtaaaactaaacataaccagcaaacgcctgaacgacaagctcgtgtagtctacaacaagattgccagccaagcgttagtagataagttacattttactgccgaagaaaacaaagttctagcagccatcaatgaattggcgcattctcaaaaagggtggggcgagtttaacatgctagatactaccaatacgtggcctagccaatag

SEQ ID NO: 502 - protein, 88 aa, Carnobacterium maltaromaticum
MIKDEKINKIYALVKSALDNTDVKNDKKLSLLLMRIQETSINGELFYDYKKELQPAISMYSIQHNFRVPDDLVKLLALVQTPKAWSGF

SEQ ID NO: 503 - DNA, 267 bp, Carnobacterium maltaromaticum
  source 1..267: mol_type = genomic DNA
atgataaaagatgaaaaaataaataaaatctatgctttagttaagagcgcacttgataatacggatgttaagaatgataaaaaactttctttacttcttatgagaatacaagaaacatcaattaatggagaactattttacgattataaaaaagaattacagccagctattagtatgtactctattcaacataactttcgggttcctgacgatctagtaaaactgttagcattagttcaaacacctaaagcttggtcagggttttaa

SEQ ID NO: 504 - protein, 111 aa, Carnobacterium maltaromaticum
MDIKSQTLYLNLSEAYKDPEVKANEFLSKLVVQCAGKLTASNSENSYIEVISLLSRGISSYYLSHKRIIPSSMLTIYTQIQKDIKNGNIDTEKLRKYEIAKGLMSVPYIYF

SEQ ID NO: 505 - DNA, 336 bp, Carnobacterium maltaromaticum
  source 1..336: mol_type = genomic DNA
atggatataaagtctcaaacattatatttgaatctaagcgaggcatataaagaccctgaagtaaaagctaatgaattcttatcaaaattagttgtacaatgtgctgggaaattaacagcttcaaacagtgagaacagttatattgaagtaatatcattgctatctaggggtatttctagttattatttatcccataaacgtataattccttcaagtatgttaactatatatactcaaatacaaaaggatataaaaaacgggaatattgacaccgaaaaattaaggaaatatgagatagcaaaaggattaatgtccgttccttatatatatttctaa

SEQ ID NO: 506 - protein, 245 aa, Lactococcus lactis
  source 1..245: note = subsp. lactis
MRRYLILIVALIGITGLSGCYQTSHKKVRFDEGSYTNFIYDNKSYFVTDKEIPQENVNNSKVKFYKLLIVDMKSEKLLSSSNKNSVTLVLNNIYEASDKSLCMGINDRYYKILPESDKGAVKALRLQNFDVTSDISDDNFVIDKNDSRKIDYMGNIYSISDTTVSDEELGEYQDVLAEVRVFDSVSGKSIPRSEWGRIDKDGSNSKQSRTEWDYGEIHSIRGKSLTEAFAVEINDDFKLATKVGN

SEQ ID NO: 507 - DNA, 738 bp, Lactococcus lactis
  source 1..738: mol_type = genomic DNA; note = subsp. lactis
atgagaagatatttaatacttattgtggccttaatagggataacaggtttatcagggtgttatcaaacaagtcataaaaaggtgaggtttgacgaaggaagttatactaattttatttatgataataaatcgtatttcgtaactgataaggagattcctcaggagaacgttaacaattccaaagtaaaattttataagctgttgattgttgacatgaaaagtgagaaacttttatcaagtagcaacaaaaatagtgtgactttggtcttaaataatatttatgaggcttctgacaagtcgctatgtatgggtattaacgacagatactataagatacttccagaaagtgataagggggcggtcaaagctttgagattacaaaactttgatgtgacaagcgatatttctgatgataattttgttattgataaaaatgattcacgaaaaattgactatatgggaaatatttacagtatatcggacaccaccgtatctgatgaagaattgggagaatatcaggatgttttagctgaagtacgtgtgtttgattcagttagtggcaaaagtatcccgaggtctgaatgggggagaattgataaggatggttcaaattccaaacagagtaggacggaatgggattatggcgaaatccattctattagaggaaaatctcttactgaagcatttgccgttgagataaatgatgattttaagcttgcaacgaaggtaggaaactag

SEQ ID NO: 508 - protein, 261 aa, Rhizobium leguminosarum
  source 1..261: note = bv. trifolii
MNDEICLTGGGRTTVTRRGGVVYREGGPWSSTVISLLRHLEASGFAEAPSVVGTGFDERGRETLSFIEGEFVHPGPWSEEAFPQFGMMLRRLHDATASFKPPENSMWRDWFGRNLGEGQHVIGHCDTGPWNIVCRSGLPVGLIDWEVAGPVRADIELAQACWLNAQLYDDDIAERVGLGSVTMRAHQVRLLLDGYGLSRKQRGGFVDKLITFAVHDAAEQAKEAAVTPESNDAEPLWAIAWRTRSASWMLHHRQTLEAALA

SEQ ID NO: 509 - DNA, 786 bp, Rhizobium leguminosarum
  source 1..786: mol_type = genomic DNA; note = bv. trifolii
atgaatgatgagatttgcctgacaggtggcggacgaacgactgtcacgcggcgcggcggagtcgtgtatcgcgaaggcggcccgtggtcatcaaccgtcatttcgctcctgcggcatctggaagcctctggcttcgctgaagctccttccgttgtcggcaccggtttcgatgagcgcggccgggagacattatcgtttatcgagggtgagtttgttcacccaggcccttggtcggaggaggcttttccgcaatttggaatgatgttgcggcgactgcacgatgccaccgcctcgttcaaacctcccgaaaactcgatgtggcgcgattggttcgggcgtaacctcggtgagggtcaacacgtaataggacactgcgacacaggcccatggaacattgtttgccggtcaggattgcctgtcgggttgatagattgggaggtggctgggcctgtcagggcggatatcgaattggcccaggcttgttggctgaatgcccagctctacgatgacgacattgcggagagggtcggattaggctctgtgaccatgagagcgcatcaagttcgcctgctgcttgacggctatggtctgtctcggaagcaacgcggcggcttcgtcgacaagctaatcacgttcgcagttcacgatgcggccgagcaggcgaaagaggcggctgtcacgccagagtcgaacgatgcggaaccgctatgggcaattgcctggcgcactagaagtgcctcctggatgctccatcatcggcaaacactggaagcagcgctggcatag

SEQ ID NO: 510 - protein, 436 aa, Bacillus subtilis
MNNIIPIMSLLFKQLYSRQGKKDAIRIAAGLVILAVFEIGLIRQAGIDESVLRKTYIILALLLMNTYMVFLSVTSQWKESYMKLSCLLPISSRSFWLAQSVVLFVDTCLRRTLFFFILPLFLFGNGTLSGAQTLFWLGRFSFFTVYSIIFGVVLSNHFVKKKNLMFLLHAAIFACVCISAALMPAATIPLCAVHILWAVVIDFPVFLQAPPQQGKMHSFMRRSEFSFYKREWNRFISSKAMLLNYAVMAVFSGFFSFQMMNTGIFNQQVIYIVISALLLICSPIALLYSIEKNDRMLLITLPIKRKTMFWAKYRFYSGLLAGGFLLVVMIVGFISGRSISVLTFLQCIELLLAGAYIRLTADEKRPSFSWQTEQQLWSGFSKYRSYLFCLPLFLAILAGTAVSLAVIPIAGLVIVYYLQKQDGGFFDTSKRERLGS

SEQ ID NO: 511 - DNA, 1000 bp, Bacillus subtilis
  source 1..1000: mol_type = genomic DNA
atgaataacataatccctatcatgtctttgctgttcaaacagctttacagccggcaagggaaaaaggacgccatccgcattgccgcaggccttgtcattctggccgtgtttgaaatcgggctgatccgccaggccggcattgatgaatcggtgttgcgcaaaacgtatatcatactcgcgcttcttttgatgaacacatatatggtgtttctttccgtgacatcacaatggaaggaatcttatatgaagctgagctgcctgctgccgatttcttcacggagcttttggctcgcccagagtgtcgttttgtttgtcgatacctgtttgagaagaactttattcttttttattttaccgctgttcttatttggaaacggaacgctgtcaggggcgcaaacattgttttggctcggcaggttttcgttttttaccgtttactccattattttcggagttgtgctaagcaaccacttcgtcaaaaagaagaacttgatgtttctgctgcatgcggcgatattcgcctgtgtatgtatcagcgccgctttgatgccggccgccacgattccgctttgcgcggttcatatcctgtgggcggtggtcattgactttcctgtctttctgcaggcgcctccgcagcagggcaagatgcattcatttatgcggcgatctgaattttcgttttacaaaagagaatggaaccgatttatctcttctaaagcgatgctgttaaattacgcggtaatggcggtattcagcggcttcttttcgttccagatgatgaacaccggcatcttcaatcagcaagtgatttatatcgtgatttccgcgcttttgctcatctgctcgccgatcgcccttttgtattcgattgaaaaaaatgaccggatgctgctcatcacgcttccgatcaagcgaaaaacgatgttttgggcgaaatatcgcttttattcaggcctattggcaggcggatttctccttgtcgtgatgattgtgggtttca

SEQ ID NO: 512 - protein, 239 aa, Bacillus subtilis
MSILDIHDVSVWYERDNVILEQVDLHLEKGAVYGLLGVNGAGKTTLINTLTGVNRNFSGRFTLCGIEAEAGMPQKTSDQLKTHRYFAADYPLLFTEITAKDYVSFVHSLYQKDFSEQQFASLAEAFHFSKYINRRISELSLGNRQKVVLMTGLLLRAPLFILDEPLVGLDVESIEVFYQKMREYCEAGGTILFSSHLLDVVQRFCDYAAILHNKQIQKVIPIGEETDLRREFFEVIGHE

SEQ ID NO: 513 - DNA, 716 bp, Bacillus subtilis
  source 1..716: mol_type = genomic DNA
gcattttggatatacacgatgtatccgtttggtatgaacgggacaacgtcatcttagagcacgtggacttacacttagaaaaaggcgccgtttacggattgcttggggtaaacggtgccggcaaaacaacactgatcaatacgctgacaggagtgaaccgcaattacagcgggggctttacgctgtgcggcattgaagctgaggccggcatgccgcagaaaacatcagatcaactgaagattcaccgttacttcgccgctgattatccgctgctgtttacagaaattacggcgaaggactatgtgtctttcgtccattcgctttatcaaaaggatttttcagagcgacagtttgccagtttggctgaggcctttcatttttcaaaatacatcaacaggagaatctcggagctgtccttggggaacaggcaaaaggttgtgttgatgacaggattattgctgcgggctcccctgtttattttggatgagccgctcgtcggtttggatgtggaatcaatagaggtcttttatcagaaaatgcgggagtactgtgaggaaggcggaaccattttgttttcttcccatctgctcgatgtcgtgcagagattttgtgattttgcggccattctgcacaacaaacagatccaaaaggtcattccgattggggaggagaccgatctgcggcgggaattttttgaggttatcggccatgaataa

SEQ ID NO: 514 - protein, 53 aa, Bacillus subtilis
MSPAQRRILLYILSFIFVIGAVVYFVKSDYLFTLIFIAIAILFGMRARKADSR

SEQ ID NO: 515 - DNA, 162 bp, Bacillus subtilis
  source 1..162: mol_type = genomic DNA
ttgtcaccagcacaaagaagaattttactgtatatcctttcatttatctttgtcatcggcgcagtcgtctattttgtcaaaagcgattatctgtttacgctgattttcattgccattgccattctgttcgggatgcgcgcgcggaaggctgactcgcgatga

SEQ ID NO: 516 - protein, 87 aa, Escherichia coli
MELKNSISDYTEAEFVQLLKEIEKENVAATDDVLDVLLEHFVKITEHPDGTDLIYYPSDNRDDSPEGIVKEIKEWRAANGKPGFKQG

SEQ ID NO: 517 - DNA, 264 bp, Escherichia coli
  source 1..264: mol_type = genomic DNA
atggaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggctga

SEQ ID NO: 518 - protein, 87 aa, Pseudomonas aeruginosa
MKSKISEYTEKEFLEFVEDIYTNNKKKFPTEESHIQAVLEFKKLTEHPSGSDLLYYPNENREDSPAGVVKEVKEWRASKGLPGFKAG

SEQ ID NO: 519 - DNA, 264 bp, Pseudomonas aeruginosa
  source 1..264: mol_type = genomic DNA
atgaagtccaagatttccgaatatacggaaaaagagtttcttgagtttgttgaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 520 - protein, 87 aa, Pseudomonas aeruginosa
MKSKISEYTEKEFLEFVKDIYTNNKKKFPTEESHIQAVLEFKKLTEHPSGSDLLYYPNENREDSPAGVVKEVKEWRASKGLPGFKAG

SEQ ID NO: 521 - DNA, 264 bp, Pseudomonas aeruginosa
  source 1..264: mol_type = genomic DNA
atgaagtccaagatttccgaatatacggaaaaagagtttcttgagtttgttaaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 522 - protein, 95 aa, Enterococcus hirae
MDFTKEEKLLNAISKVYNEATIDDYPDLKEKLFLYSKEISEGKSVGEVSMKLSSFLGRYILKHKFGLPKSLIELQEIVSKESQVYRGWASIGIWS

SEQ ID NO: 523 - DNA, 288 bp, Enterococcus hirae
  source 1..288: mol_type = genomic DNA
atggattttactaaagaagaaaaacttttaaatgcaattagtaaagtatacaatgaagcaactatagatgactatcctgacttaaaagaaaagctctttctttattctaaagaaatcagtgagggaaaaagtgttggtgaagttagtatgaaattaagtagttttcttggaagatatattttaaaacataaatttggattacctaaatctttaatagaattacaagaaattgttagtaaggaatctcaagtatatagaggatgggcttctattggtatttggagttaa

SEQ ID NO: 524 - protein, 113 aa, Leuconostoc mesenteroides
MKKKYRYLEDSKNYTSTLYSLLVDNVDKPGYSDICDVLLQVSKKLDNTQSVEALINRLVNYIRITASTYKIIFSKKEEELIIKLGVIGQKAGLNGQYMADFSDKSQFYSVFDQ

SEQ ID NO: 525 - DNA, 342 bp, Leuconostoc mesenteroides
  source 1..342: mol_type = genomic DNA
ttgaaaaaaaagtatcggtatttagaagatagcaaaaattacactagtacactctattctctgttagttgataatgttgacaaacctggatactcagatatttgcgatgttttgcttcaagtttctaagaagttggataatactcaaagtgttgaagcgctaattaatcgattggttaattatattcgtattactgcttcaacatacaaaattattttttcaaaaaaagaagaggaattgattataaaacttggtgttattggacaaaaagctggacttaatggtcagtatatggctgatttttcagacaagtctcagttttacagcgttttcgatcagtaa

SEQ ID NO: 526 - protein, 93 aa, Escherichia coli
MSFLNFAFSPVFFSIMACYFIVWRNKRNEFVCNRLLSIIIISFLICFIYPWLNYKIEVKYYIFEQFYLFCFLSSLVAVVINLIVYFILYRRCI

SEQ ID NO: 527 - DNA, 282 bp, Escherichia coli
  source 1..282: mol_type = genomic DNA
atgagttttcttaattttgcattttctcctgtattcttctccattatggcgtgttatttcattgtatggagaaataaacgaaacgaatttgtctgcaatagattgctatcaattataataatatcttttttgatatgcttcatatatccatggctaaattacaaaatcgaagttaaatattatatatttgaacagttttatcttttttgttttttatcgtcactcgtggctgttgtaataaacctaattgtatactttatattatacaggagatgtatatga

SEQ ID NO: 528 - protein, 96 aa, Escherichia coli
MHLKYYLHNLPESLIPWILILIFNDNDNTPLLFIFISSIHVLLYPYSKLTISRYIKENTKLKKEPWYLCKLSALFYLLMAIPVGLPSFIYYTLKRN

SEQ ID NO: 529 - DNA, 291 bp, Escherichia coli
  source 1..291: mol_type = genomic DNA
atgcatttaaaatactacctacataatttacctgaatcacttataccatggattcttattttaatatttaacgacaatgataacactcctttgttatttatatttatatcatcaatacatgtattgctatatccatactctaaattaaccatatctagatatatcaaagaaaatacaaagttaaaaaaagaaccctggtacttatgcaagttatctgcattgttttatttattaatggcaatcccagtaggattgccaagtttcatatattacactctaaagagaaattaa

SEQ ID NO: 530 - protein, 344 aa, Escherichia coli
MMIQSHPLLAAPLAVGDTIGFFSSSAPATVTAKNRFFRGVEFLQRKGFKLVSGKLTGKTDFYRSGTIKERAQEFNELVYNPDITCIMSTIGGDNSNSLLPFLDYDAIIANPKIIIGYSDTTALLAGIYAKTGLITFYGPALIPSFGEHPPLVDITYESFIKILTRKQSGIYTYTLPEKWSDESINWNENKILRPKKLYKNNCAFYGSGKVEGRVIGGNLNTLTGIWGSEWMPEILNGDILFIEDSRKSIATIERLFSMLKLNRVFDKVSAIILGKHELFDCAGSKRRPYEVLTEVLDGKQIPVLDGFDCSHTHPMLTLPLGVKLAIDFDNKNISITEQYLSTEK

SEQ ID NO: 531 - DNA, 1000 bp, Escherichia coli
  source 1..1000: mol_type = genomic DNA
atgatgatacaatctcatccactactggccgctcccctggcagtaggagatacaattggtttcttttcatcatctgctccggcaacagttactgcaaaaaatcgtttttttcggggagttgagtttcttcagagaaagggatttaagctggtatcagggaagcttaccggtaaaacagatttttatcgttcaggtactattaaagaaagagctcaagaatttaatgagttagtctacaatcctgatattacctgtataatgtcaacgatcggtggagataacagtaattcactactaccgtttctggactatgatgctatcattgcaaaccccaaaattatcataggttactcagatacaactgctttattagcaggaatatatgcaaaaacagggttaataacattctatggaccagctcttattccttcgtttggtgaacatccacctcttgtggatataacatatgaatcatttattaaaatactaacaagaaaacaatcaggaatatatacctacacattacctgaaaagtggagtgatgagagcataaactggaatgaaaacaagatattaaggcctaagaagctatataaaaacaactgtgccttttatggttccggaaaagttgaggggcgtgtaattggaggaaatctaaatactttgacaggtatatgggggagtgaatggatgcctgaaattcttaatggagatatattgtttattgaggacagtcggaaaagcattgcaacaattgaacgattattctctatgctaaagcttaatcgcgtgtttgataaagttagtgcaataatactcgggaaacatgagctttttgattgtgcaggaagtaaacgcagaccatatgaagtattaacagaggtattagatgggaaacagattcctgtactggatggatttgattgttcacatacacatccaatgctaactcttccacttggtgtaaaattagctattgactttgacaacaaaaatatat

SEQ ID NO: 532 - protein, 90 aa, Lactobacillus sakei
MKADYKKINSILTYTSTALKNPKIIKDKDLVVLLTIIQEEAKQNRIFYDYKRKFRPAVTRFTIDNNFEIPDCLVKLLSAVETPKAWSGFS

SEQ ID NO: 533 - DNA, 268 bp, Lactobacillus sakei
  source 1..268: mol_type = genomic DNA
ggcagattataaaaaaataaattcaatactaacttacacatctactgctttaaaaaaccctaaaattataaaagataaagatttagtagtccttctaactattattcaagaagaagccaaacaaaatagaatcttttatgattataaaagaaaatttcgtccagcggttactcgctttacaattgataataattttgagattcctgattgtttggttaaactactgtcagctgttgaaacacctaaggcgtggtctggatttagttag

SEQ ID NO: 534 - protein, 83 aa, Escherichia coli
MKLSPKAAIEVCNEAAKKGLWILGIDGGHWLNPGFRIDSSASWTYDMPEEYKSKTPENNRLAIENIKDDIENGYTAFIITLKM

SEQ ID NO: 535 - DNA, 251 bp, Escherichia coli
  source 1..251: mol_type = genomic DNA
tgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggaggaatacaaatcaaaaacccctgaaaataatagattggctattgaaaatattaaagatgatattgagaatggatacactgctttcattatcacgttaaagatgtaa

SEQ ID NO: 536 - protein, 436 aa, Bacillus subtilis
MNNIFPIMSLLFKQLYSRQGKKDAIRIAAGLVILAVFEIGLIRQAGIDESVLGKTYIILALLLMNTYMVFLSVTSQWKESYMKLSCLLPISSRSFWLAQSVVLFVDTCLRRTLFFFILPLFLFGNGTLSGAQTLFWLGRFSFFTVYSILFGVMLSNHFVKKKNSMFLLHAAVFAFVCLSAAFMPAVTIPLCAVHMLWAVIIDFPVFLQAPPHQSKMHFFMRRSEFSFYKREWNRFISSKAMLLNYVVMAAFSGFFSFQMMNTGIFNQQVIYIVISALLLICSPIALLYSIEKNDRMLLITLPIKRRTMFWAKYRFYSGLLAGGFLLVAIIVGFISGRPISALTFVQCMELLLAGAFIRLTADEKRPSFGWQTEQQLWSGFSKYRSYLFCLPLFLATLAGTAVSLAVIPIAALIIVYYLQKQDGGFFDTSKRERIGS

SEQ ID NO: 537 - DNA, 1000 bp, Bacillus subtilis
  source 1..1000: mol_type = genomic DNA
ttggggaggagaccgatctgcggcgggaattttttgaggttatcggccatgaataacatattccccatcatgtcgttgctgttcaaacagctgtacagccggcaagggaaaaaggacgctatccgcattgctgcagggcttgtgattctcgccgtgtttgaaatcgggctgatccgacaagccggcattgacgaatcggtgttgggaaaaacgtatatcatattggcgcttctcttaatgaacacgtatatggtgtttctttccgtgacatcacaatggaaggaatcttatatgaagctgagctgtctgctgccgatttcatcacggagcttttggctcgcccagagtgtcgttctgtttgtcgatacctgtttgagaagaacgttattcttttttattttaccgctgttcttatttggaaacggaacgctgtcaggggcgcaaacattgttttggcttggcagattttcgttttttaccgtttactcgattctattcggagttatgctaagcaaccatttcgtcaaaaagaagaactcgatgtttctgctgcatgcggcggtattcgcctttgtatgcctcagtgccgcttttatgccggccgtcacgatcccgctatgcgcggttcacatgctatgggcggtgatcattgactttccggtctttctgcaggcgcctccgcatcagagcaagatgcatttttttatgcggcgatctgaattttcgttttacaaaagagaatggaaccgatttatttcttctaaagcgatgctgttaaattacgtggtgatggcggcgttcagcggattcttttcgttccagatgatgaacactggcatcttcaatcagcaagtgatttatattgtgatttccgctctattgctgatttgctcgccgatcgcccttttgtactctattgaaaaaaacgatcgcatgctgctcatcacgcttccaattaaaagaagaacgatgttttgggcgaaatatcgcttttattcag

SEQ ID NO: 538 - protein, 580 aa, Escherichia coli
MERKQKNSLFNYIYSLMDVRGKFLFFSMLFITSLSSIIISISPLILAKITDLLSGSLSNFSYEYLVLLACLYMFCVISNKASVFLFMILQSSLRINMQKKMSLKYLRELYNENITNLSKNNAGYTTQSLNQASNDIYILVRNVSQNILSPVIQLISTIVVVLSTKDWFSAGVFFLYILVFVIFNTRLTGSLASLRKHSMDITLNSYSLLSDTVDNMIAAKKNNALRLISERYEDALTQENNAQKKYWLLSSKVLLLNSLLAVILFGSVFIYNILGVLNGVVSIGHFIMITSYIILLSTPVENIGALLSEIRQSMSSLAGFIQRHAENKATSPSIPFLNMERKLNLSIRELSFSYSDDKKILNSVSLDLFTGKMYSLTGPSGSGKSTLVKIISGYYKNYFGDIYLNDISLRNISDEDLNDAIYYLTQDDYIFMDTLRFNLRLANYDASENEIFKVLKLANLSVVNNEPVSLDTHLINRGNNYSGGQKQRISLARLFLRKPAIIIIDEATSALDYINESEILSSIRTHFPDALIINISHRINLLECSDCVYVLNEGNIVASGHFRDLMVSNEYISGLASVTE

SEQ ID NO: 539 - DNA, 1000 bp, Escherichia coli
  source 1..1000: mol_type = genomic DNA
atggaaagaaaacagaaaaactcattatttaattatatttattcattaatggatgtaagaggtaaatttttattcttttccatgttattcattacatcattatcatcgataatcatatctatttcaccattgattcttgcaaagattacagatttactgtctggctcattgtcaaattttagttatgaatatctggttttacttgcctgtttatacatgttttgcgttatatctaataaagcaagtgtttttttatttatgatactgcaaagtagtctacgtattaacatgcagaaaaaaatgtcgctaaagtatttgagagaattgtataacgaaaatataactaacttgagtaaaaataatgctggatatacaacgcaaagtcttaaccaggcttcaaatgacatttatattcttgtgagaaatgtttcccagaatatcctgtcacctgttatacaacttatttccactattgttgttgttttatctacgaaggactggttttctgccggtgtgttttttctctatattctggtatttgtaatttttaataccagactgactggcagtttagcgtctctcagaaaacacagcatggatatcactcttaactcttatagtctgttatctgatactgttgataacatgatagcagctaaaaagaataatgcattaagacttatttctgaacgttatgaagatgctctcactcaggaaaacaatgctcagaaaaaatactggttactcagttctaaagttcttttattgaactctttacttgctgtaatattatttggttctgtattcatatataatattttaggtgtgctgaatggtgtagttagtatcggccacttcattatgattacatcatatatcattcttctttcaacgccagtggaaaatataggggcattgctaagtgagatcaggcagtcaatgtctagcctggcaggttttattcaacgtcatgccgagaataaagccacatctccttcaa

SEQ ID NO: 540 - protein, 95 aa, Klebsiella pneumoniae
MTLLSFGFSPVFFSVMAFCIISRSKFYPQRTRNKVIVLILLTFFICFLYPLTKVYLVGSYGIFDKFYLFCFISTLIAIAINVVILTINGAKNERN

SEQ ID NO: 541 - DNA, 288 bp, Klebsiella pneumoniae
  source 1..288: mol_type = genomic DNA
atgacattactttcatttggattttctcctgttttcttttcagtcatggcgttctgtatcatttcacgtagtaaattctatccgcagagaacgcgaaacaaagttattgttctgattttactaactttttttatttgttttttatatccattaacaaaagtgtatctggtgggaagttacggtatatttgacaaattctacctcttttgctttatttctacgttaattgcaatagcaattaacgtagtgatacttacaataaatggagctaagaatgagagaaattag

SEQ ID NO: 542 - RNA, 13 nt, synthetic construct
  misc_feature 1..13: note = Kozak sequence
  source 1..13: mol_type = other RNA
gccgccrccatgg

SEQ ID NO: 543 - skipped

SEQ ID NO: 544 - DNA, 30 bp, synthetic construct
  misc_feature 1..30: note = Lead promoter
  source 1..30: mol_type = other DNA
gaaaaccttgtcaatgaagagcgatctatg

SEQ ID NO: 545 - DNA, 30 bp, synthetic construct
  misc_feature 1..30: note = FecA promoter
  source 1..30: mol_type = other DNA
ttctcgttcgactcatagctgaacacaaca

SEQ ID NO: 546 - DNA, 16 bp, synthetic construct
  misc_feature 1..16: note = Cu-sensitive promoter
  source 1..16: mol_type = other DNA
atgacaaaattgtcat

SEQ ID NO: 547 - DNA, 30 bp, synthetic construct
  misc_feature 1..30: note = Fe promoter
  source 1..30: mol_type = other DNA
accaatgctgggaacggccagggcacctaa

SEQ ID NO: 548 - DNA, 30 bp, synthetic construct
  misc_feature 1..30: note = Fe and UV promoters
  source 1..30: mol_type = other DNA
ctgaaagcgcataccgctatggagggggtt

SEQ ID NO: 549 - DNA, 30 bp, synthetic construct
  misc_feature 1..30: note = PrFe (PI + PII rus operon)
  source 1..30: mol_type = other DNA
tagatatgcctgaaagcgcataccgctatg

SEQ ID NO: 550 - DNA, 30 bp, synthetic construct
  misc_feature 1..30: note = Lux cassette right promoter
  source 1..30: mol_type = other DNA
tgttatagtcgaatacctctggcggtgata

SEQ ID NO: 551 - DNA, 30 bp, synthetic construct
  misc_feature 1..30: note = P(Las) TetO
  source 1..30: mol_type = other DNA
ttttggtacactccctatcagtgatagaga

SEQ ID NO: 552 - DNA, 30 bp, synthetic construct
  misc_feature 1..30: note = P(Las) CIO
  source 1..30: mol_type = other DNA
ctttttggtacactacctctggcggtgata

SEQ ID NO: 553 - DNA, 30 bp, synthetic construct
  misc_feature 1..30: note = P(Rhl)
  source 1..30: mol_type = other DNA
tacgcaagaaaatggtttgttatagtcgaa

SEQ ID NO: 554 - DNA, 30 bp, synthetic construct
  misc_feature 1..30: note = Double Promoter (LuxR/HSL, positive / cI,negative)
  source 1..30: mol_type = other DNA
cgtgcgtgttgataacaccgtgcgtgttga

SEQ ID NO: 555 - DNA, 30 bp, synthetic construct
  misc_feature 1..30: note = P2 promoter in agr operon from S. aureus
  source 1..30: mol_type = other DNA
agattgtactaaatcgtataatgacagtga

SEQ ID NO: 556 - DNA, 30 bp, synthetic construct
  misc_feature 1..30: note = plux-cI hybrid promoter
  source 1..30: mol_type = other DNA
gtgttgatgcttttatcaccgccagtggta

SEQ ID NO: 557 - DNA, 30 bp, synthetic construct
  misc_feature 1..30: note = plux-lac hybrid promoter
  source 1..30: mol_type = other DNA
agtgtgtggaattgtgagcggataacaatt

SEQ ID NO: 558 - DNA, 30 bp, synthetic construct
  misc_feature 1..30: note = CinR, CinL and glucose controlled promotor
  source 1..30: mol_type = other DNA
acatcttaaaagttttagtatcatattcgt

SEQ ID NO: 559 - DNA, 30 bp, synthetic construct; identical to SEQ ID NO: 553
  misc_feature 1..30: note = RhIR promoter repressible by CI
  source 1..30: mol_type = other DNA

SEQ ID NO: 560 - DNA, 30 bp, synthetic construct
  misc_feature 1..30: note = Reverse Lux Promoter
  source 1..30: mol_type = other DNA
tcttgcgtaaacctgtacgatcctacaggt

SEQ ID NO: 561 - DNA, 30 bp, synthetic construct
  misc_feature 1..30: note = rhlI promoter
  source 1..30: mol_type = other DNA
atcctcctttagtcttccccctcatgtgtg

SEQ ID NO: 562 - DNA, 30 bp, synthetic construct
  misc_feature 1..30: note = lasI promoter
  source 1..30: mol_type = other DNA
taaaattatgaaatttgcataaattcttca

SEQ ID NO: 563 - DNA, 30 bp, synthetic construct
  misc_feature 1..30: note = LuxR+3OC6HSL independent R0065
  source 1..30: mol_type = other DNA
gtgttgactattttacctctggcggtgata

SEQ ID NO: 564 - DNA, 30 bp, synthetic construct
  misc_feature 1..30: note = LasR/LasI Inducible & RHLR/RHLI repressiblePromoter
  source 1..30: mol_type = other DNA
gaaatctggcagtttttggtacacgaaagc

SEQ ID NO: 565 - DNA, 30 bp, synthetic construct
  misc_feature 1..30: note = pLux/cI Hybrid Promoter
  source 1..30: mol_type = other DNA
acaccgtgcgtgttgatatagtcgaataaa

SEQ ID NO: 566 - DNA, 30 bp, synthetic construct
  misc_feature 1..30: note = pLas promoter
  source 1..30: mol_type = other DNA
aaaattatgaaatttgtataaattcttcag

SEQ ID NO: 567 - DNA, 30 bp, synthetic construct
  misc_feature 1..30: note = pLas/cI Hybrid Promoter
  source 1..30: mol_type = other DNA
ggttctttttggtacctctggcggtgataa

SEQ ID NO: 568 - DNA, 30 bp, synthetic construct
  misc_feature 1..30: note = pLas/Lux Hybrid Promoter
  source 1..30: mol_type = other DNA
tgtaggatcgtacaggtataaattcttcag

SEQ ID NO: 569 - DNA, 30 bp, synthetic construct
  misc_feature 1..30: note = pLux
  source 1..30: mol_type = other DNA
caagaaaatggtttgttatagtcgaataaa

SEQ ID NO: 570 - DNA, 30 bp, synthetic construct
  misc_feature 1..30: note = pLux/Las Hybrid Promoter
  source 1..30: mol_type = other DNA
ctatctcatttgctagtatagtcgaataaa

SEQ ID NO: 571 - DNA, 30 bp, synthetic construct
  misc_feature 1..30: note = Hybrid promoter: HSL-LuxR activated, P22 C2repressed
  source 1..30: mol_type = other DNA
tagtttataatttaagtgttctttaatttc

SEQ ID NO: 572 - DNA, 30 bp, synthetic construct
  misc_feature 1..30: note = PAI+LasR -> LuxI (AI)
  source 1..30: mol_type = other DNA
caccttcgggtgggcctttctgcgtttata

SEQ ID NO: 573 - DNA, 30 bp, synthetic construct
  misc_feature 1..30: note = PAI+LasR -> LasI & AI+LuxR --| LasI
  source 1..30: mol_type = other DNA
aataactctgatagtgctagtgtagatctc

SEQ ID NO: 574 - DNA, 30 bp, synthetic construct; identical to SEQ ID NO: 572
  misc_feature 1..30: note = PAI+LasR -> LasI+GFP & AI+LuxR --| LasI+GFP
  source 1..30: mol_type = other DNA

SEQ ID NO: 575 - DNA, 30 bp, synthetic construct; identical to SEQ ID NO: 572
  misc_feature 1..30: note = Complex QS -> LuxI & LasI circuit
  source 1..30: mol_type = other DNA

SEQ ID NO: 576 - DNA, 30 bp, synthetic construct; identical to SEQ ID NO: 569
  misc_feature 1..30: note = position 3 mutated promoter lux pR-3 (luxR & HSLregulated)
  source 1..30: mol_type = other DNA

SEQ ID NO: 577 - DNA, 30 bp, synthetic construct; identical to SEQ ID NO: 569
  misc_feature 1..30: note = position 5 mutated promoter lux pR-5 (luxR & HSLregulated)
  source 1..30: mol_type = other DNA

SEQ ID NO: 578 - DNA, 30 bp, synthetic construct; identical to SEQ ID NO: 569
  misc_feature 1..30: note = position 3&5 mutated promoter lux pR-3/5 (luxR &HSL regulated)
  source 1..30: mol_type = other DNA

SEQ ID NO: 579 - DNA, 30 bp, synthetic construct
  misc_feature 1..30: note = Promoter (HSL-mediated luxR repressor)
  source 1..30: mol_type = other DNA
ttgacacctgtaggatcgtacaggtataat

SEQ ID NO: 580 - DNA, 30 bp, synthetic construct; identical to SEQ ID NO: 569
  misc_feature 1..30: note = Promoter (luxR & HSL regulated -- lux pR)
  source 1..30: mol_type = other DNA

SEQ ID NO: 581 - DNA, 30 bp, synthetic construct
  misc_feature 1..30: note = Promoter (luxR & HSL regulated -- lux pL)
  source 1..30: mol_type = other DNA
cacgcaaaacttgcgacaaacaataggtaa

SEQ ID NO: 582 - DNA, 30 bp, synthetic construct
  misc_feature 1..30: note = Promoter (RhlR & C4-HSL regulated)
  source 1..30: mol_type = other DNA
gttagctttcgaattggctaaaaagtgttc

SEQ ID NO: 583 - DNA, 30 bp, synthetic construct
  misc_feature 1..30: note = Promoter (cinR and HSL regulated)
  source 1..30: mol_type = other DNA
ccattctgctttccacgaacttgaaaacgc

SEQ ID NO: 584 - DNA, 30 bp, synthetic construct
  misc_feature 1..30: note = Promoter (LasR & PAI regulated)
  source 1..30: mol_type = other DNA
ggccgcgggttctttttggtacacgaaagc

SEQ ID NO: 585 - DNA, 30 bp, synthetic construct
  misc_feature 1..30: note = Promoter, Standard (luxR and HSL regulated -- luxpR)
  source 1..30: mol_type = other DNA
aagaaaatggtttgttgatactcgaataaa

SEQ ID NO: 586 - DNA, 30 bp, synthetic construct
  misc_feature 1..30: note = P(Bla)
  source 1..30: mol_type = other DNA
gtttatacataggcgagtactctgttatgg

SEQ ID NO: 587 - DNA, 30 bp, synthetic construct
  misc_feature 1..30: note = P(Cat)
  source 1..30: mol_type = other DNA
agaggttccaactttcaccataatgaaaca

SEQ ID NO: 588 - DNA, 30 bp, synthetic construct
  misc_feature 1..30: note = P(Kat)
  source 1..30: mol_type = other DNA
taaacaactaacggacaattctacctaaca

SEQ ID NO: 589 - DNA, 30 bp, synthetic construct
  misc_feature 1..30: note = Template for Building Primer Family Member
  source 1..30: mol_type = other DNA
acatcaagccaaattaaacaggattaacac

SEQ ID NO: 590 - DNA, 30 bp, synthetic construct
  misc_feature 1..30: note = Reverse lambda cI-regulated promoter
  source 1..30: mol_type = other DNA
gaggtaaaatagtcaacacgcacggtgtta

SEQ ID NO: 591 - DNA, 30 bp, synthetic construct
  misc_feature 1..30: note = Key Promoter absorbs 3
  source 1..30: mol_type = other DNA
caggccggaataactccctataatgcgcca

SEQ ID NO: 592 - DNA, 30 bp, synthetic construct
  misc_feature 1..30: note = constitutive promoter family member
  source 1..30: mol_type = other DNA
ggctagctcagtcctaggtacagtgctagc

SEQ ID NO: 593 - DNA, 30 bp, synthetic construct
  misc_feature 1..30: note = constitutive promoter family member
  source 1..30: mol_type = other DNA
agctagctcagtcctaggtattatgctagc

SEQ ID NO: 594 - DNA, 30 bp, synthetic construct
  misc_feature 1..30: note = constitutive promoter family member
  source 1..30: mol_type = other DNA
agctagctcagtcctaggtactgtgctagc

SEQ ID NO: 595 - DNA, 30 bp, synthetic construct
  misc_feature 1..30: note = constitutive promoter family member
  source 1..30: mol_type = other DNA
agctagctcagtcctagggattatgctagc

SEQ ID NO: 596 - DNA, 30 bp, synthetic construct
  misc_feature 1..30: note = constitutive promoter family member
  source 1..30: mol_type = other DNA
agctagctcagtcctaggtattgtgctagc

SEQ ID NO: 597 - DNA, 30 bp, synthetic construct
  misc_feature 1..30: note = constitutive promoter family member
  source 1..30: mol_type = other DNA
ggctagctcagtcctaggtactatgctagc

SEQ ID NO: 598 - DNA, 30 bp, synthetic construct
  misc_feature 1..30: note = constitutive promoter family member
  source 1..30: mol_type = other DNA
ggctagctcagtcctaggtatagtgctagc

SEQ ID NO: 599 - DNA, 30 bp, synthetic construct
  misc_feature 1..30: note = constitutive promoter family member
  source 1..30: mol_type = other DNA
ggctagctcagccctaggtattatgctagc

SEQ ID NO: 600 - DNA, 30 bp, synthetic construct
  misc_feature 1..30: note = constitutive promoter family member
  source 1..30: mol_type = other DNA
agctagctcagtcctaggtataatgctagc

SEQ ID NO: 601 - DNA, 30 bp, synthetic construct
  misc_feature 1..30: note = constitutive promoter family member
  source 1..30: mol_type = other DNA
agctagctcagtcctagggactgtgctagc

SEQ ID NO: 602 - DNA, 30 bp, synthetic construct
  misc_feature 1..30: note = constitutive promoter family member
  source 1..30: mol_type = other DNA
ggctagctcagtcctaggtacaatgctagc

SEQ ID NO: 603 - DNA, 30 bp, synthetic construct; identical to SEQ ID NO: 598
  misc_feature 1..30: note = constitutive promoter family member
  source 1..30: mol_type = other DNA

SEQ ID NO: 604 - DNA, 30 bp, synthetic construct; identical to SEQ ID NO: 595
  misc_feature 1..30: note = constitutive promoter family member
  source 1..30: mol_type = other DNA

SEQ ID NO: 605 - DNA, 30 bp, synthetic construct
  misc_feature 1..30: note = constitutive promoter family member
  source 1..30: mol_type = other DNA
ggctagctcagtcctagggattatgctagc

SEQ ID NO: 606 - DNA, 30 bp, synthetic construct; identical to SEQ ID NO: 602
  misc_feature 1..30: note = constitutive promoter family member
  source 1..30: mol_type = other DNA

SEQ ID NO: 607 - DNA, 30 bp, synthetic construct
  misc_feature 1..30: note = constitutive promoter family member
  source 1..30: mol_type = other DNA
agctagctcagcccttggtacaatgctagc

SEQ ID NO: 608 - DNA, 30 bp, synthetic construct
  misc_feature 1..30: note = constitutive promoter family member
  source 1..30: mol_type = other DNA
agctagctcagtcctagggactatgctagc

SEQ ID NO: 609 - DNA, 30 bp, synthetic construct
  misc_feature 1..30: note = constitutive promoter family member
  source 1..30: mol_type = other DNA
agctagctcagtcctagggattgtgctagc

SEQ ID NO: 610 - DNA, 30 bp, synthetic construct
  misc_feature 1..30: note = constitutive promoter family member
  source 1..30: mol_type = other DNA
ggctagctcagtcctaggtattgtgctagc

SEQ ID NO: 611 - DNA, 30 bp, synthetic construct; identical to SEQ ID NO: 600
  misc_feature 1..30: note = constitutive promoter family member
  source 1..30: mol_type = other DNA

SEQ ID NO: 612 - DNA, 30 bp, synthetic construct
  misc_feature 1..30: note = 1bp mutant from J23107
  source 1..30: mol_type = other DNA
ggctagctcagtcctaggtattatgctagc

SEQ ID NO: 613 - DNA, 30 bp, synthetic construct; identical to SEQ ID NO: 602
  misc_feature 1..30: note = 1bp mutant from J23114
  source 1..30: mol_type = other DNA

SEQ ID NO: 614 - DNA, 30 bp, synthetic construct
  misc_feature 1..30: note = pBAD reverse
  source 1..30: mol_type = other DNA
aaagtgtgacgccgtgcaaataatcaatgt

SEQ ID NO: 615 - DNA, 30 bp, synthetic construct
  misc_feature 1..30: note = NikR promoter, a protein of the ribbon helix-helixfamily of trancription factors that repress expre
  source 1..30: mol_type = other DNA
gacgaatacttaaaatcgtcatacttattt

SEQ ID NO: 616 - DNA, 30 bp, synthetic construct
  misc_feature 1..30: note = lacq_Promoter
  source 1..30: mol_type = other DNA
aaacctttcgcggtatggcatgatagcgcc

SEQ ID NO: 617 - DNA, 30 bp, synthetic construct
  misc_feature 1..30: note = lacIQ - promoter sequence
  source 1..30: mol_type = other DNA
tgatagcgcccggaagagagtcaattcagg

SEQ ID NO: 618 - DNA, 30 bp, synthetic construct
  misc_feature 1..30: note = E. Coli CreABCD phosphate sensing operon promoter
  source 1..30: mol_type = other DNA
ttatttaccgtgacgaactaattgctcgtg

SEQ ID NO: 619 - DNA, 30 bp, synthetic construct
  misc_feature 1..30: note = GlnRS promoter
  source 1..30: mol_type = other DNA
catacgccgttatacgttgtttacgctttg

SEQ ID NO: 620 - DNA, 30 bp, synthetic construct
  misc_feature 1..30: note = Constitutive weak promoter of lacZ
  source 1..30: mol_type = other DNA
ttatgcttccggctcgtatgttgtgtggac

SEQ ID NO: 621 - DNA, 30 bp, synthetic construct
  misc_feature 1..30: note = Mutated LacZ promoter
  source 1..30: mol_type = other DNA
ttatgcttccggctcgtatggtgtgtggac

SEQ ID NO: 622 - DNA, 30 bp, synthetic construct
  misc_feature 1..30: note = constitutive promoter with (TA)10 between -10 and-35 elements
  source 1..30: mol_type = other DNA
atatatatatatatataatggaagcgtttt

SEQ ID NO: 623 - DNA, 30 bp, synthetic construct; identical to SEQ ID NO: 622
  misc_feature 1..30: note = constitutive promoter with (TA)9 between -10 and-35 elements
  source 1..30: mol_type = other DNA

SEQ ID NO: 624 - DNA, 30 bp, synthetic construct
  misc_feature 1..30: note = constitutive promoter with (C)10 between -10 and-35 elements
  source 1..30: mol_type = other DNA
ccccgaaagcttaagaatataattgtaagc

SEQ ID NO: 625 - DNA, 30 bp, synthetic construct; identical to SEQ ID NO: 624
  misc_feature 1..30: note = constitutive promoter with (C)12 between -10 and-35 elements
  source 1..30: mol_type = other DNA

SEQ ID NO: 626 - DNA, 30 bp, synthetic construct
  misc_feature 1..30: note = optimized (TA) repeat constitutive promoter with13 bp between -10 and -35 elements
  source 1..30: mol_type = other DNA
tgacaatatatatatatatataatgctagc

SEQ ID NO: 627 - DNA, 30 bp, synthetic construct
  misc_feature 1..30: note = optimized (TA) repeat constitutive promoter with15 bp between -10 and -35 elements
  source 1..30: mol_type = other DNA
acaatatatatatatatatataatgctagc

SEQ ID NO: 628 - DNA, 30 bp, synthetic construct
  misc_feature 1..30: note = optimized (TA) repeat constitutive promoter with17 bp between -10 and -35 elements
  source 1..30: mol_type = other DNA
aatatatatatatatatatataatgctagc

SEQ ID NO: 629 - DNA, 30 bp, synthetic construct
  misc_feature 1..30: note = optimized (TA) repeat constitutive promoter with19 bp between -10 and -35 elements
  source 1..30: mol_type = other DNA
tatatatatatatatatatataatgctagc

SEQ ID NO: 630 - DNA, 30 bp, synthetic construct; identical to SEQ ID NO: 629
  misc_feature 1..30: note = optimized (TA) repeat constitutive promoter with21 bp between -10 and -35 elements
  source 1..30: mol_type = other DNA

SEQ ID NO: 631 - DNA, 30 bp, synthetic construct
  misc_feature 1..30: note = optimized (A) repeat constitutive promoter with 17bp between -10 and -35 elements
  source 1..30: mol_type = other DNA
aaaaaaaaaaaaaaaaaatataatgctagc

SEQ ID NO: 632 - DNA, 30 bp, synthetic construct; identical to SEQ ID NO: 631
  misc_feature 1..30: note = optimized (A) repeat constitutive promoter with 18bp between -10 and -35 elements
  source 1..30: mol_type = other DNA

SEQ ID NO: 633 - DNA, 30 bp, synthetic construct; identical to SEQ ID NO: 572
  misc_feature 1..30: note = J23101:GFP
  source 1..30: mol_type = other DNA

SEQ ID NO: 634 - DNA, 30 bp, synthetic construct; identical to SEQ ID NO: 572
  misc_feature 1..30: note = J23119:IFP
  source 1..30: mol_type = other DNA

SEQ ID NO: 635 - DNA, 30 bp, synthetic construct; identical to SEQ ID NO: 572
  misc_feature 1..30: note = J23119:HO1
  source 1..30: mol_type = other DNA

SEQ ID NO: 636 - DNA, 30 bp, synthetic construct; identical to SEQ ID NO: 572
  misc_feature 1..30: note = Infrared signal reporter (J23119:IFP:J23119:HO1)
  source 1..30: mol_type = other DNA

SEQ ID NO: 637 - DNA, 30 bp, synthetic construct; identical to SEQ ID NO: 592
  misc_feature 1..30: note = Double terminator + constitutive promoter
  source 1..30: mol_type = other DNA

SEQ ID NO: 638 - DNA, 30 bp, synthetic construct
  misc_feature 1..30: note = Double terminator + Constitutive promoter + StrongRBS
  source 1..30: mol_type = other DNA
tgctagctactagagattaaagaggagaaa

SEQ ID NO: 639 - DNA, 30 bp, synthetic construct
  misc_feature 1..30: note = IPTG inducible Lac promoter cassette
  source 1..30: mol_type = other DNA
ttgtgagcggataacaagatactgagcaca

SEQ ID NO: 640 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 641 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 642 - DNA, 30 bp, synthetic construct
  misc_feature 1..30: note = M13K07 gene I promoter
  source 1..30: mol_type = other DNA
cctgtttttatgttattctctctgtaaagg

SEQ ID NO: 643 - DNA, 30 bp, synthetic construct
  misc_feature 1..30: note = M13K07 gene II promoter
  source 1..30: mol_type = other DNA
aaatatttgcttatacaatcttcctgtttt

SEQ ID NO: 644 - DNA, 30 bp, synthetic construct
  misc_feature 1..30: note = M13K07 gene III promoter
  source 1..30: mol_type = other DNA
gctgataaaccgatacaattaaaggctcct

SEQ ID NO: 645 - DNA, 30 bp, synthetic construct
  misc_feature 1..30: note = M13K07 gene IV promoter
  source 1..30: mol_type = other DNA
ctcttctcagcgtcttaatctaagctatcg

SEQ ID NO: 646 - DNA, 30 bp, synthetic construct
  misc_feature 1..30: note = M13K07 gene V promoter
  source 1..30: mol_type = other DNA
atgagccagttcttaaaatcgcataaggta

SEQ ID NO: 647 - DNA, 30 bp, synthetic construct
  misc_feature 1..30: note = M13K07 gene VI promoter
  source 1..30: mol_type = other DNA
ctattgattgtgacaaaataaacttattcc

SEQ ID NO: 648 - DNA, 30 bp, synthetic construct
  misc_feature 1..30: note = M13K07 gene VIII promoter
  source 1..30: mol_type = other DNA
gtttcgcgcttggtataatcgctgggggtc

SEQ ID NO: 649 - DNA, 30 bp, synthetic construct
  misc_feature 1..30: note = M13110
  source 1..30: mol_type = other DNA
ctttgcttctgactataatagtcagggtaa

SEQ ID NO: 650 - DNA, 30 bp, synthetic construct
  misc_feature 1..30: note = Modified promoter sequence of g3.
  source 1..30: mol_type = other DNA
aaaccgatacaattaaaggctcctgctagc

SEQ ID NO: 651 - DNA, 30 bp, synthetic construct
  misc_feature 1..30: note = Constitutive Promoter I
  source 1..30: mol_type = other DNA
caccacactgatagtgctagtgtagatcac

SEQ ID NO: 652 - DNA, 30 bp, synthetic construct
  misc_feature 1..30: note = Constitutive Promoter II
  source 1..30: mol_type = other DNA
gccggaataactccctataatgcgccacca

SEQ ID NO: 653 - DNA, 30 bp, synthetic construct
  misc_feature 1..30: note = --Specify Parts List--
  source 1..30: mol_type = other DNA
ttgacaagcttttcctcagctccgtaaact

SEQ ID NO: 654 - DNA, 30 bp, synthetic construct
  misc_feature 1..30: note = Full-length stationary phase osmY promoter
  source 1..30: mol_type = other DNA
ggtttcaaaattgtgatctatatttaacaa

SEQ ID NO: 655 - DNA, 30 bp, synthetic construct; identical to SEQ ID NO: 654
  misc_feature 1..30: note = Minimal stationary phase osmY promoter
  source 1..30: mol_type = other DNA

SEQ ID NO: 656 - DNA, 30 bp, synthetic construct
  misc_feature 1..30: note = htpG Heat Shock Promoter
  source 1..30: mol_type = other DNA
tctattccaataaagaaatcttcctgcgtg

SEQ ID NO: 657 - DNA, 30 bp, synthetic construct
  misc_feature 1..30: note = Promoter veg a constitutive promoter for B.subtilis
  source 1..30: mol_type = other DNA
aaaaatgggctcgtgttgtacaataaatgt

SEQ ID NO: 658 - DNA, 30 bp, synthetic construct
  misc_feature 1..30: note = Promoter 43 a constitutive promoter for B.subtilis
  source 1..30: mol_type = other DNA
aaaaaaagcgcgcgattatgtaaaatataa

SEQ ID NO: 659 - DNA, 30 bp, synthetic construct
  misc_feature 1..30: note = Strong constitutive promoter for Bacillus subtilis
  source 1..30: mol_type = other DNA
aattgcagtaggcatgacaaaatggactca

SEQ ID NO: 660 - DNA, 30 bp, synthetic construct
  misc_feature 1..30: note = PliaG
  source 1..30: mol_type = other DNA
caagcttttcctttataatagaatgaatga

SEQ ID NO: 661 - DNA, 30 bp, synthetic construct
  misc_feature 1..30: note = PlepA
  source 1..30: mol_type = other DNA
tctaagctagtgtattttgcgtttaatagt

SEQ ID NO: 662 - DNA, 30 bp, synthetic construct
  misc_feature 1..30: note = Pveg
  source 1..30: mol_type = other DNA
aatgggctcgtgttgtacaataaatgtagt

SEQ ID NO: 663 - DNA, 30 bp, synthetic construct
  misc_feature 1..30: note = Promoter ctc for B. subtilis
  source 1..30: mol_type = other DNA
atccttatcgttatgggtattgtttgtaat

SEQ ID NO: 664 - DNA, 30 bp, synthetic construct
  misc_feature 1..30: note = Promoter gsiB for B. subtilis
  source 1..30: mol_type = other DNA
taaaagaattgtgagcgggaatacaacaac

SEQ ID NO: 665 - DNA, 30 bp, synthetic construct; identical to SEQ ID NO: 658
  misc_feature 1..30: note = Promoter 43 a constitutive promoter for B.subtilis
  source 1..30: mol_type = other DNA

SEQ ID NO: 666 - DNA, 30 bp, synthetic construct
  misc_feature 1..30: note = Pspv2 from Salmonella
  source 1..30: mol_type = other DNA
tacaaaataattcccctgcaaacattatca

SEQ ID NO: 667 - DNA, 30 bp, synthetic construct
  misc_feature 1..30: note = Pspv from Salmonella
  source 1..30: mol_type = other DNA
tacaaaataattcccctgcaaacattatcg

SEQ ID NO: 668 - DNA, 30 bp, synthetic construct
  misc_feature 1..30: note = T7 promoter (strong promoter from T7bacteriophage)
  source 1..30: mol_type = other DNA
agggaatacaagctacttgttctttttgca

SEQ ID NO: 669 - DNA, 23 bp, synthetic construct
  misc_feature 1..23: note = T7 Promoter
  source 1..23: mol_type = other DNA
taatacgactcactatagggaga

SEQ ID NO: 670 - DNA, 28 bp, synthetic construct
  misc_feature 1..28: note = T7 Promoter
  source 1..28: mol_type = other DNA
gaatttaatacgactcactatagggaga

SEQ ID NO: 671 - DNA, 19 bp, synthetic construct
  misc_feature 1..19: note = T7 consensus -10 and rest
  source 1..19: mol_type = other DNA
taatacgactcactatagg

SEQ ID NO: 672 - DNA, 30 bp, synthetic construct
  misc_feature 1..30: note = overlapping T7 promoter
  source 1..30: mol_type = other DNA
gagtcgtattaatacgactcactatagggg

SEQ ID NO: 673 - DNA, 30 bp, synthetic construct
  misc_feature 1..30: note = more overlapping T7 promoter
  source 1..30: mol_type = other DNA
agtgagtcgtactacgactcactatagggg

SEQ ID NO: 674 - DNA, 30 bp, synthetic construct
  misc_feature 1..30: note = weaken overlapping T7 promoter
  source 1..30: mol_type = other DNA
gagtcgtattaatacgactctctatagggg

SEQ ID NO: 675 - DNA, 23 bp, synthetic construct
  misc_feature 1..23: note = T7 Consensus Promoter Sequence
  source 1..23: mol_type = other DNA
taatacgactcactatagggaga

SEQ ID NO: 676 - DNA, 23 bp, synthetic construct
  misc_feature 1..23: note = T7 RNAP promoter
  source 1..23: mol_type = other DNA
ttatacgactcactatagggaga

SEQ ID NO: 677 - DNA, 23 bp, synthetic construct
  misc_feature 1..23: note = T7 RNAP promoter
  source 1..23: mol_type = other DNA
gaatacgactcactatagggaga

SEQ ID NO: 678 - DNA, 23 bp, synthetic construct
  misc_feature 1..23: note = T7 RNAP promoter
  source 1..23: mol_type = other DNA
taatacgtctcactatagggaga

SEQ ID NO: 679 - DNA, 23 bp, synthetic construct
  misc_feature 1..23: note = T7 RNAP promoter
  source 1..23: mol_type = other DNA
tcatacgactcactatagggaga

SEQ ID NO: 680 - DNA, 30 bp, synthetic construct
  misc_feature 1..30: note = T7 strong promoter
  source 1..30: mol_type = other DNA
taatacgactcactatagggagaccacaac

SEQ ID NO: 681 - DNA, 30 bp, synthetic construct
  misc_feature 1..30: note = T7 weak binding and processivity
  source 1..30: mol_type = other DNA
taattgaactcactaaagggagaccacagc

SEQ ID NO: 682 - DNA, 30 bp, synthetic construct
  misc_feature 1..30: note = T7 weak binding promoter
  source 1..30: mol_type = other DNA
cgaagtaatacgactcactattagggaaga

SEQ ID NO: 683 - DNA, 30 bp, synthetic construct
  misc_feature 1..30: note = pCyc (Medium) Promoter
  source 1..30: mol_type = other DNA
acaaacacaaatacacacactaaattaata

SEQ ID NO: 684 - DNA, 30 bp, synthetic construct
  misc_feature 1..30: note = pAdh (Strong) Promoter
  source 1..30: mol_type = other DNA
ccaagcatacaatcaactatctcatataca

SEQ ID NO: 685 - DNA, 30 bp, synthetic construct
  misc_feature 1..30: note = pSte5 (Weak) Promoter
  source 1..30: mol_type = other DNA
gatacaggatacagcggaaacaacttttaa

SEQ ID NO: 686 - DNA, 30 bp, synthetic construct
  misc_feature 1..30: note = yeast ADH1 promoter
  source 1..30: mol_type = other DNA
tttcaagctataccaagcatacaatcaact

SEQ ID NO: 687 - DNA, 30 bp, synthetic construct
  misc_feature 1..30: note = cyc100 minimal promoter
  source 1..30: mol_type = other DNA
cctttgcagcataaattactatacttctat

SEQ ID NO: 688 - DNA, 30 bp, synthetic construct; identical to SEQ ID NO: 687
  misc_feature 1..30: note = cyc70 minimal promoter
  source 1..30: mol_type = other DNA

SEQ ID NO: 689 - DNA, 30 bp, synthetic construct; identical to SEQ ID NO: 687
  misc_feature 1..30: note = cyc43 minimal promoter
  source 1..30: mol_type = other DNA

SEQ ID NO: 690 - DNA, 30 bp, synthetic construct; identical to SEQ ID NO: 687
  misc_feature 1..30: note = cyc28 minimal promoter
  source 1..30: mol_type = other DNA

SEQ ID NO: 691 - DNA, 30 bp, synthetic construct; identical to SEQ ID NO: 687
  misc_feature 1..30: note = cyc16 minimal promoter
  source 1..30: mol_type = other DNA

SEQ ID NO: 692 - DNA, 30 bp, synthetic construct
  misc_feature 1..30: note = pPGK1
  source 1..30: mol_type = other DNA
ttatctactttttacaacaaatataaaaca

SEQ ID NO: 693 - DNA, 30 bp, synthetic construct; identical to SEQ ID NO: 683
  misc_feature 1..30: note = pCYC Yeast Promoter
  source 1..30: mol_type = other DNA

SEQ ID NO: 694 - DNA, 30 bp, synthetic construct
  misc_feature 1..30: note = Yeast GPD (TDH3) Promoter
  source 1..30: mol_type = other DNA
gtttcgaataaacacacataaacaaacaaa

SEQ ID NO: 695 - DNA, 30 bp, synthetic construct; identical to SEQ ID NO: 684
  misc_feature 1..30: note = yeast mid-length ADH1 promoter
  source 1..30: mol_type = other DNA

SEQ ID NO: 696 - DNA, 30 bp, synthetic construct
  misc_feature 1..30: note = Yeast CLB1 promoter region, G2/M cell cyclespecific
  source 1..30: mol_type = other DNA
accatcaaaggaagctttaatcttctcata

SEQ ID NO: 697 - DNA, 30 bp, synthetic construct
  misc_feature 1..30: note = CMV promoter
  source 1..30: mol_type = other DNA
agaacccactgcttactggcttatcgaaat

SEQ ID NO: 698 - DNA, 30 bp, synthetic construct
  misc_feature 1..30: note = Ubc Promoter
  source 1..30: mol_type = other DNA
ggccgtttttggcttttttgttagacgaag

SEQ ID NO: 699 - protein, 9 aa, Lactobacillus curvatus
NIPQLTPTP

SEQ ID NO: 700 - DNA, 27 bp, Lactobacillus curvatus
  source 1..27: mol_type = genomic DNA
aacattccgcagctgaccccgaccccg

SEQ ID NO: 701 - protein, 18 aa, Bacillus thuringiensis
  VARIANT 4..10: note = X= any amino acid
DWTXWSXLVXAACSVELL

SEQ ID NO: 702 - DNA, 54 bp, Bacillus thuringiensis; identical to SEQ ID NO: 389
  source 1..54: mol_type = genomic DNA
  variation 10..30: note = n = A,T,C or G

SEQ ID NO: 703 - protein, 30 aa, Lactobacillus curvatus; identical to SEQ ID NO: 390
  source 1..30: note = L442

SEQ ID NO: 704 - DNA, 90 bp, Lactobacillus curvatus; identical to SEQ ID NO: 391
  source 1..90: mol_type = genomic DNA; note = L442

SEQ ID NO: 705 - protein, 43 aa, Carnobacterium divergens; identical to SEQ ID NO: 392

SEQ ID NO: 706 - DNA, 129 bp, Carnobacterium divergens; identical to SEQ ID NO: 393
  source 1..129: mol_type = genomic DNA

SEQ ID NO: 707 - protein, 24 aa, Microbispora sp.
  source 1..24: note = strain 107891
VTSWSLCTPGCTSPGGGSNCSFCC

SEQ ID NO: 708 - DNA, 72 bp, Microbispora sp.
  source 1..72: mol_type = genomic DNA; note = strain 107891
gtgaccagctggagcctgtgcaccccgggctgcaccagcccgggcggcggcagcaactgcagcttttgctgc

SEQ ID NO: 709 - protein, 62 aa, Enterococcus sp.; identical to SEQ ID NO: 394

SEQ ID NO: 710 - DNA, 186 bp, Enterococcus sp.; identical to SEQ ID NO: 395
  source 1..186: mol_type = genomic DNA

SEQ ID NO: 711 - protein, 39 aa, Enterococcus faecium; identical to SEQ ID NO: 396

SEQ ID NO: 712 - DNA, 117 bp, Enterococcus faecium; identical to SEQ ID NO: 397
  source 1..117: mol_type = genomic DNA

SEQ ID NO: 713 - protein, 30 aa, Paenibacillus polymyxa; identical to SEQ ID NO: 398

SEQ ID NO: 714 - DNA, 90 bp, Paenibacillus polymyxa; identical to SEQ ID NO: 399
  source 1..90: mol_type = genomic DNA

SEQ ID NO: 715 - protein, 31 aa, Staphylococcus epidermidis; identical to SEQ ID NO: 400

SEQ ID NO: 716 - DNA, 93 bp, Staphylococcus epidermidis; identical to SEQ ID NO: 401
  source 1..93: mol_type = genomic DNA

SEQ ID NO: 717 - protein, 58 aa, Enterococcus faecium
MEKLTVKEMSQVVGGKYYGNGVSCNKKGCSVDWGKAIGIIGNNAAANLTTGGKAGWKG

SEQ ID NO: 718 - DNA, 178 bp, Enterococcus faecium
  source 1..178: mol_type = genomic DNA
atggaaaaattaactgtgaaagaaatgtcgcaagtagttggcggaaagtactatggtaacggagtatcatgtaataaaaagggatgtagtgttgattggggaaaagctattggtattattggaaataatgctgctgctaatttaactactggcggaaaagcagggtggaaaggttaac

SEQ ID NO: 719 - protein, 39 aa, Paenibacillus polymyxa; identical to SEQ ID NO: 404

SEQ ID NO: 720 - DNA, 93 bp, Paenibacillus polymyxa; identical to SEQ ID NO: 401
  source 1..93: mol_type = genomic DNA

SEQ ID NO: 721 - protein, 35 aa, Bacillus circulans; identical to SEQ ID NO: 406

SEQ ID NO: 722 - DNA, 105 bp, Bacillus circulans; identical to SEQ ID NO: 407
  source 1..105: mol_type = genomic DNA

SEQ ID NO: 723 - protein, 30 aa, Paenibacillus polymyxa; identical to SEQ ID NO: 408

SEQ ID NO: 724 - DNA, 90 bp, Paenibacillus polymyxa; identical to SEQ ID NO: 409
  source 1..90: mol_type = genomic DNA

SEQ ID NO: 725 - protein, 13 aa, Lactobacillus rhamnosus
AVPAVRKTNETLD

SEQ ID NO: 726 - DNA, 39 bp, Lactobacillus rhamnosus; identical to SEQ ID NO: 411
  source 1..39: mol_type = genomic DNA

SEQ ID NO: 727 - protein, 60 aa, Bacillus licheniformis
MKNSAAREAFKGANHPAGMVSEEELKALVGGNDVNPETTPATTSSWTCITAGVTVSASLC

SEQ ID NO: 728 - DNA, 180 bp, Bacillus licheniformis
  source 1..180: mol_type = genomic DNA
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgc

SEQ ID NO: 729 - protein, 689 aa, Pseudomonas aeruginosa; identical to SEQ ID NO: 276

SEQ ID NO: 730 - DNA, 2070 bp, Pseudomonas aeruginosa; identical to SEQ ID NO: 277
  source 1..2070: mol_type = genomic DNA

SEQ ID NO: 731 - protein, 36 aa, Lactobacillus plantarum; identical to SEQ ID NO: 414

SEQ ID NO: 732 - DNA, 108 bp, Lactobacillus plantarum; identical to SEQ ID NO: 415
  source 1..108: mol_type = genomic DNA

SEQ ID NO: 733 - protein, 30 aa, Lactococcus lactis
MKTILRFVAGYDIASHKKKTGGYPWERGKA

SEQ ID NO: 734 - DNA, 93 bp, Lactococcus lactis
  source 1..93: mol_type = genomic DNA
atgaaaacaatcctacgttttgttgctggctacgatattgctagtcataaaaagaaaactggcggctatccatgggaacgtggaaaagcttaa

SEQ ID NO: 735 - protein, 24 aa, Lactobacillus acidophilus
GNPKVAHCASQIGRSTAWGAVSGA

SEQ ID NO: 736 - DNA, 72 bp, Lactobacillus acidophilus; identical to SEQ ID NO: 417
  source 1..72: mol_type = genomic DNA

SEQ ID NO: 737 - protein, 53 aa, Lactobacillus salivarius
  source 1..53: note = cp400
MFFNFMKKVDVKKNFGYKEVSRKDLAKVNGGKRKKHRCRVYNNGMPTGMYRWC

SEQ ID NO: 738 - DNA, 162 bp, Lactobacillus salivarius
  source 1..162: mol_type = genomic DNA; note = cp400
atgttttttaattttatgaaaaaagtagatgtgaagaagaattttggatataaagaagtttctagaaaagatctagctaaagtaaatggtggaaagagaaagaaacatcgttgcagagtttataataatggaatgcctacaggaatgtatcgttggtgctaa

SEQ ID NO: 739 - protein, 5 aa, synthetic construct
  REGION 1..5: note = Caspase 2 consensus sequence
DVADL

SEQ ID NO: 740 - protein, 5 aa, synthetic construct
  REGION 1..5: note = Caspase 2 consensus sequence
DVADI

SEQ ID NO: 741 - protein, 12 aa, synthetic construct
  REGION 1..12: note = Synthetic RAP-binding peptide, RBP
FHWWQTSPAHFS

SEQ ID NO: 742 - protein, 12 aa, synthetic construct
  REGION 1..12: note = Synthetic RAP-binding peptide, RBP
WPFAHWPWQYPR

SEQ ID NO: 743 - protein, 9 aa, synthetic construct
  REGION 1..9: note = Synthetic AgrC ligand
  SITE 5..9: note = Thiolacton linkage between C5 and F9
GDSVCASYF

SEQ ID NO: 744 - protein, 7 aa, synthetic construct
  REGION 1..7: note = Synthetic AgrC ligand
  SITE 3..7: note = Thiolacton linkage between C5 and F9
SVCASYF

SEQ ID NO: 745 - protein, 5 aa, synthetic construct
  REGION 1..5: note = Synthetic Cry1Aa ligand
SKADT

SEQ ID NO: 746 - protein, 5 aa, synthetic construct
  REGION 1..5: note = Synthetic Cry1Aa ligand
SKPAD

SEQ ID NO: 747 - protein, 11 aa, synthetic construct
  REGION 1..11: note = Synthetic Fsr ligand
  SITE 3..11: note = Thiolacton linkage between C5 and F9
QNSAAAFAAWA

SEQ ID NO: 748 - protein, 11 aa, synthetic construct
  REGION 1..11: note = Synthetic Fsr ligand
  SITE 3..11: note = Thiolacton linkage between C5 and F9
QNSAAAFGQWA

SEQ ID NO: 749 - protein, 7 aa, synthetic construct
  REGION 1..7: note = Synthetic AgrC1, AgrC2
  SITE 4..7: note = Thiolacton linkage between C5 and F9
YSTCFIM

SEQ ID NO: 750 - protein, 7 aa, synthetic construct
  REGION 1..7: note = Synthetic AgrC1, AgrC2
  SITE 3..7: note = Thiolacton linkage between C5 and F9
STCAFIM